SEQUENCE LISTING (3 sequences)

SEQ ID NO: 1 - DNA, 131 bp, Homo sapiens
  source 1..131: mol_type = genomic DNA
tgtttaggatgccccaggaatgtgcagatctgcgttggaagggtagaaacgggaatgaaagcaaatggtaaggatccatggaacgaaatatggggtggagatagcagcgtttccaccaaacttctatcgaa

SEQ ID NO: 2 - DNA, 142 bp, synthetic construct
  source 1..142: mol_type = other DNA
atgaaagcaaatggtnnggatccatggaacgaaatatggggtggagatagcagcgtttccaccaaacttctatcgaactttaggatgccccaggantgtgcagatctgcgttggaagggtagaaacgggnnnnnnnnnnnnn

SEQ ID NO: 3 - DNA, 142 bp, synthetic construct
  source 1..142: mol_type = other DNA
nnnnnnnnnnnnnatgaaagcaaatggtaaggatccatggaacgaaatatggggtggaganagcagcgtntccaccaaacttctatcgaagtttaggatgccccaggaatgtgcagatctgcgttggaagggtagaaacggg

(19) United States
(12) Patent Application Publication
(10) Pub. No.: US 2026/0258502 A1
(43) Pub. Date: Sep. 3, 2026

(54) METHODS AND REAGENTS FOR DETECTION OF CIRCULAR DNA MOLECULES IN BIOLOGICAL SAMPLES

(71) Applicant: TWINSTRAND BIOSCIENCES, INC., Seattle, WA (US)

(72) Inventors: Devon Marie FITZGERALD, Seattle, WA (US); Jacob Emerson HIGGINS, Seattle, WA (US); Isabel Yisao LEE, Seattle, WA (US); Jesse SALK, Seattle, WA (US)

(73) Assignee: TWINSTRAND BIOSCIENCES, INC., Seattle, WA (US)

(21) Appl. No.: 19/107,457

(22) PCT Filed: Aug. 29, 2023
(86) PCT No.: PCT/US2023/073119

§ 371 Date: Feb. 27, 2025

Related U.S. Application Data
(60) Provisional application No. 63/373,851, filed on Aug. 29, 2022.
(60) Provisional application No. 63/384,066, filed on Nov. 16, 2022.

(51) Int. Cl. C12Q 1/6886

(52) U.S. Cl. CPC C12Q 1/6886; C12Q 2600/156

(57) ABSTRACT

The present disclosure relates to methods and associated reagents, kits, and systems for detecting, quantifying, and characterizing extrachromosomal circular DNA (eccDNA) molecules in biological samples using error-corrected sequencing methods such as Duplex Sequencing (DS). The present disclosure also provides methods for preparing sequencing libraries that can be used for the detection of eccDNAs.

### CROSS-REFERENCE TO RELATED APPLICATIONS

This application claims priority to U.S. Provisional Application No. 63/384,066, filed Nov. 16, 2022, and U.S. Provisional Application No. 63/373,851, filed Aug. 29, 2022, each of which is hereby incorporated by reference in its entirety.

### REFERENCE TO SEQUENCE LISTING SUBMITTED ELECTRONICALLY

The instant application contains a Sequence Listing which has been submitted electronically in XML filed format and is hereby incorporated by reference in its entirety. Said XML copy, created on Aug. 29, 2023, is named “TSB-018SeqList.XML” and is 4,047 bytes in size.

### TECHNICAL FIELD

The present technology relates generally to methods for preparing and analyzing nucleic acid libraries, e.g., DNA libraries for next-generation sequencing (NGS) applications such as duplex sequencing. In particular, some embodiments of the technology are directed to detecting and/or quantifying extrachromosomal circular DNA (eccDNA) molecules in biological samples.

### BACKGROUND

Extrachromosomal circular DNA (eccDNA) molecules can be found in the nuclei of eukaryotic cells and vary in size from less than 100 bp to several megabases. eccDNA molecules can contain any element found in the human genome from small, noncoding regions to entire genes. During mitosis, eccDNA molecules may be maintained, but due to a lack of centromeres, they are not segregated evenly during cell division.

eccDNA molecules have implications in human health. For example, cancers commonly harbor eccDNA molecules that drive tumorigenesis. Additionally, elevated levels of eccDNA molecules are present in urinary cell-free samples from individuals with chronic kidney disease. Thus, detection of eccDNA can be a marker of human disease.

There is therefore a need for new methods for detecting and quantifying non-linear molecules such as eccDNAs in sequencing libraries obtained from biological samples, and to preparing libraries from biological samples in ways that maximize the preservation of such molecules. The present disclosure addresses this need and provides other advantages as well.

### SUMMARY

In one aspect, the present disclosure provides a method of detecting candidate extrachromosomal circular DNA (eccDNA) molecules in a biological sample, the method comprising: (a) providing a sequencing library comprising a plurality of double-stranded DNA fragments obtained from the sample; (b) obtaining error-corrected sequences for double-stranded DNA fragments in the library; (c) detecting possible insertions in the double-stranded DNA fragments by aligning a plurality of the error-corrected sequences with a reference genome; (d) detecting putative eccDNA breakpoints in one or more of the fragments in which a possible insertion has been detected, wherein a putative eccDNA breakpoint comprises a sequence B located upstream of a sequence A, wherein: (i) sequence A is present upstream of sequence B in the reference genome; (ii) the first nucleotide of sequence A is located distance of Y nucleotides upstream of the last nucleotide of sequence B in the reference genome; and (iii) the last nucleotide of sequence B is located approximately immediately upstream of the first nucleotide of sequence A in the putative eccDNA breakpoint; (e) detecting putative eccDNA molecules among the fragments comprising a putative eccDNA breakpoint, wherein a putative eccDNA molecule is a fragment comprising a putative eccDNA breakpoint that is not excluded by any one or more of steps (i), (ii), or (iii), the steps comprising: (i) comparing the length of the fragment to the distance Y nucleotides, wherein a determination that the fragment is longer than Y nucleotides indicates that the fragment is not a putative eccDNA molecule; (ii) determining whether the error-corrected sequence for the fragment comprises a duplication of any subsequence comprised within the region from sequence A to sequence B in the reference genome, wherein a detection of a duplication indicates that the fragment is not a putative eccDNA molecule; and/or (iii) determining whether a sequence located upstream of sequence A in the reference genome is present upstream of sequence B in the error-corrected sequence for the fragment, or whether a sequence located downstream of sequence B in the reference genome is present downstream of sequence A in the error-corrected sequence for the fragment, wherein a detection that a sequence located upstream of sequence A or downstream of sequence B in the reference genome is present upstream of sequence B or downstream of sequence A, respectively, in the error-corrected sequence indicates that the fragment is not a putative eccDNA molecule.

In some embodiments of the method, the error-corrected sequences obtained in (b) are obtained using consensus sequencing. In some embodiments, the consensus sequencing is duplex sequencing (DS). In some embodiments, the consensus sequencing is single-stranded consensus sequencing (SSCS) or a combination of DS and SSCS. In some embodiments, the biological sample is selected from the group consisting of a sperm sample, semen sample, prostatic fluid sample, testicular biopsy sample, spermatogonia sample, germ cell sample, gamete sample, swab, lavage, aspirate, biopsy, tissue sample, tumor sample, preneoplastic sample, liquid biopsy, hyperplasia sample, hypertrophy sample, dysplastic sample, urine sample, CSF sample, any other body fluid sample, autopsy sample, necropsy sample, surgical sample, model organism sample, plasma sample, serum sample, gastric sample, bone marrow sample, stool sample, brushing sample, bile sample, pancreatic fluid sample, synovial fluid sample, sputum sample, mucus sample, vitreous sample, forensic sample, environmental sample, bacterial sample, fungal sample, mammalian sample, human sample, and diagnostic sample.

In some embodiments, the biological sample comprises potential cancer cells or potentially cancer-derived nucleic acids. In some embodiments, the biological sample comprises cell-free DNA. In some embodiments, the biological sample comprises cells that have been exposed to a potentially toxic agent. In some embodiments, the potentially toxic agent is a potentially clastogenic, aneugenic, mutagenic, and/or teratogenic agent. In some embodiments, the presence and/or character of eccDNA molecules in the sample is used to identify a disease state or a physiological state. In some embodiments, the disease state or physiological state is selected from the group consisting of inflammation, autoimmunity, infection, organ transplant rejection, stem cell transplant rejection, therapeutic cell rejection, therapeutic cell response, immunotherapy response, pregnancy, pre-eclampsia, radiation exposure, sun exposure, drug exposure, and hypersensitivity. In some embodiments, the double-stranded DNA fragments were obtained by enzymatic fragmentation. In some embodiments, the average length of the double-stranded DNA fragments in the library is between about 100 bp and 1000 bp. In some embodiments, the average length of the double-stranded DNA fragments in the library is greater than about 1000 bp, 2000 bp, 3000 bp, 4000 bp, 5000 bp, 6000 bp, 7000 bp, 8000 bp, 9000 bp, or 10,000 bp.

In some embodiments, the length of the putative eccDNA molecule is between about 100 and 1000 nucleotides. In some embodiments, the length of the putative eccDNA molecule is less than about 500 nucleotides. In some embodiments, the length of the putative eccDNA molecule is greater than about 1000 bp, 2000 bp, 3000 bp, 4000 bp, 5000 bp, 6000 bp, 7000 bp, 8000 bp, 9000 bp, or 10,000 bp. In some embodiments, the length of the putative eccDNA molecule is greater than about 100 kb, 200 kb, 300 kb, 400 kb, 500 kb, 600 kb, 700 kb, 800 kb, 900 kb, 1 Mb, 2 Mb, or 3 Mb. In some embodiments, the putative eccDNA molecule comprises a gene. In some embodiments, the putative eccDNA molecule comprises an origin of replication. In some embodiments, the length of the putative eccDNA molecule is approximately equal to the distance Y nucleotides. In some embodiments, the length of the putative eccDNA molecule is exactly equal to the distance Y nucleotides. In some embodiments, the length of the putative eccDNA molecule is less than about 50%, 60%, 70%, 80%, 90%, or more of the average length of the DNA fragments in the library. In some embodiments, the error-corrected sequences obtained in (b) are specific to a single genomic region. In some embodiments, the error-corrected sequences obtained in (b) are specific to from about 1 to about 30 individual genomic loci.

In some embodiments, the method is performed with or without an enrichment step to increase the proportion of double-stranded circular DNA molecules among all double-stranded nucleic acids in the sample, and further comprising: comparing the frequencies in the library of possible insertions as detected in step (c), of putative eccDNA breakpoints as detected in step (d), and/or of putative eccDNA molecules as detected in step (e), obtained with the method performed with or without the enrichment step. In some embodiments, the enrichment step comprises selectively eliminating double-stranded linear DNA molecules from the sample. In some embodiments, the double-stranded linear DNA molecules are selectively eliminated by treating the sample with one or more exonucleases. In some embodiments, the enrichment step comprises selectively isolating double-stranded circular DNA molecules from the sample. In some embodiments, the double-stranded circular DNA molecules are selectively isolated using electrophoresis, column filtration, density gradient centrifugation, selective extraction, and/or using a DNA binding protein that will differentially bind to or remain bound to double-stranded circular DNA molecules as compared to double-stranded linear DNA molecules. In some embodiments, the DNA binding protein is a helicase.

In some embodiments, the ratio of the number of putative eccDNA molecules detected in step (e) to the number of error-corrected sequences obtained in step (b), to the number of possible insertions detected in step (c), or to the number of putative eccDNA breakpoints detected in step (d), is higher when the method is performed with the enrichment step than without the enrichment step. In some embodiments, the frequency of possible insertions as detected in step (c) with the enrichment step is less than about 90%, 80%, 70%, 60%, 50%, 40%, 30%, 20%, or 10% of the frequency of possible insertions as detected in step (c) without the enrichment step. In some embodiments, the frequency of putative eccDNA breakpoints as detected in step (d) with the enrichment step is less than about 90%, 80%, 70%, 60%, 50%, 40%, 30%, 20%, or 10% of the frequency of putative eccDNA breakpoints as detected in step (d) without the enrichment step. In some embodiments, the frequency of putative eccDNA molecules as detected in step (e) with the enrichment step is at least about 10%, 20%, 30%, 40%, 50%, 60%, 70%, 80%, 90%, or more of the frequency of putative eccDNA molecules as detected in step (e) without the enrichment step. In some embodiments, e.g., where essentially all of the insertions present in a given sample correspond to eccDNA molecules, the frequencies of the different categories may not significantly decrease with enrichment of circular DNA molecules. In some embodiments, e.g., where essentially all of the insertions present in a given sample correspond to eccDNA molecules, the frequencies of the different categories may increase with enrichment of circular DNA molecules.

In some embodiments, the method further comprises performing a calculation of a probability that a putative eccDNA molecule identified in step (e) is a genuine eccDNA molecule. In some embodiments, the calculation is based in part on any one or more of the herein-disclosed frequencies, ratios, or percentages. In some embodiments, the calculation in based in part on a relationship between the length of the putative eccDNA molecule to the average length of double-stranded DNA fragments in the library, wherein a lower length of the putative eccDNA molecule relative to the average length of double-stranded DNA fragments in the library indicates a higher probability that the putative eccDNA molecule is a genuine eccDNA molecule. In some embodiments, the calculation is based in part on an observation that the length of the putative eccDNA molecule is approximately or exactly equal to the distance Y nucleotides, wherein the observation indicates a higher probability that the putative eccDNA molecule is a genuine eccDNA molecule.

In some embodiments, any one or more of steps (a)-(e), a determination of any one or more of the herein-disclosed frequencies, ratios, or percentages, or any of the herein-disclosed calculations is performed on a computer. In some embodiments, any one or more of steps (a)-(e), a determination of any one or more of the herein-disclosed frequencies, ratios, or percentages, or any of the herein-disclosed calculations is performed in the cloud.

In another aspect, the present disclosure provides a computer-based system for performing any one or more of the herein-disclosed methods.

In another aspect, the present disclosure provides a method of treating a disease or other medical condition in a mammalian subject, the method comprising: (i) performing any one of the herein-disclosed methods on a biological sample obtained from the subject; (ii) identifying one or more putative eccDNA molecules in the sample that are indicative of the disease or of a physiological state associated with the medical condition; and (iii) treating the subject for the disease or medical condition.

In another aspect, the present disclosure provides a method of preparing a sequencing library for the detection of candidate extrachromosomal circular DNA (eccDNA) molecules in a biological sample, the method comprising: (a) providing a biological sample comprising double-stranded DNA; (b) preparing a first, unenriched portion of the biological sample and a second, enriched portion of the biological sample that is enriched for double-stranded circular DNA molecules, wherein the second portion is prepared by selectively eliminating linear double-stranded DNA molecules and/or selectively isolating double-stranded circular DNA molecules from the sample; (c) fragmenting double-stranded DNA molecules in the first portion of the biological sample to produce a population of unenriched double-stranded DNA fragments, and enzymatically fragmenting double-stranded DNA molecules in the second portion of the biological sample to produce a population of enriched double-stranded DNA fragments; (d) ligating sequencing adapters to a plurality of the unenriched double-stranded DNA fragments to produce an unenriched sequencing library; and (e) ligating sequencing adapters to a plurality of the enriched double-stranded DNA fragments to produce an enriched sequencing library.

In some embodiments, the biological sample is selected from the group consisting of a sperm sample, semen sample, prostatic fluid sample, testicular biopsy sample, spermatogonia sample, germ cell sample, gamete sample, swab, lavage, aspirate, biopsy, tissue sample, tumor sample, preneoplastic sample, liquid biopsy, hyperplasia sample, hypertrophy sample, dysplastic sample, urine sample, CSF sample, any other body fluid sample, autopsy sample, necropsy sample, surgical sample, model organism sample, plasma sample, serum sample, gastric sample, bone marrow sample, stool sample, brushing sample, bile sample, pancreatic fluid sample, synovial fluid sample, sputum sample, mucus sample, vitreous sample, forensic sample, environmental sample, bacterial sample, fungal sample, mammalian sample, human sample, and diagnostic sample.

In some embodiments, the biological sample comprises potential cancer cells or potentially cancer-derived nucleic acids. In some embodiments, the biological sample comprises cell-free DNA. In some embodiments, the biological sample comprises cells that have been exposed to a potentially toxic agent. In some embodiments, the potentially toxic agent is a potentially clastogenic, aneugenic, mutagenic, and/or teratogenic agent. In some embodiments, the fragmentation is enzymatic fragmentation. In some embodiments, the second sample is enriched by treating the sample with one or more exonucleases. In some embodiments, the one or more exonucleases comprise exonuclease I, exonuclease T, exonuclease VII, exonuclease III, T7 exonuclease, exonuclease V (RecBCD), exonuclease VIII, lambda exonuclease, or T5 exonuclease.

In some embodiments, the method further comprises treating a portion of the second sample with one or more endonucleases prior to treating the sample with one or more exonucleases, and comparing the putative eccDNAs obtained in the presence or absence of treatment with the one or more endonucleases. In some embodiments, the second sample is enriched by selectively isolating double-stranded circular DNA molecules from the sample. In some embodiments, the double-stranded circular DNA molecules are selectively isolated using electrophoresis, column filtration, density gradient centrifugation, selective extraction, and/or using a DNA binding protein that will differentially bind to or remain bound to double-stranded circular DNA molecules as compared to double-stranded linear DNA molecules. In some embodiments, the DNA binding protein is a helicase. In some embodiments, the biological sample is treated with DTT prior to step (b), (c) or (d).

In some embodiments, the method further comprises: preparing a third and a fourth portion of the biological sample by removing sub-portions of the first, unenriched, and the second, enriched, portions prepared in step (b), respectively; treating a fraction of the third and a fraction of the fourth portions with a reagent that induces breaks in double-stranded circular DNA molecules at sites of DNA damage, and leaving a fraction of the third and the fourth portions untreated; ligating sequencing adapters to the treated and untreated fractions of the third and the fourth portions.

In some embodiments, the reagent is FPG (formamidopyrimidine [fapy]-DNA glycosylase) or UDG (Uracil-DNA Glycosylase) with endonuclease VIII. In some embodiments, the sequencing adapters are duplex sequencing adapters. In some embodiments, the sequencing adapters comprise a Y shape. In some embodiments, the sequencing adapters are hairpin adapters.

In another aspect, the present disclosure provides a sequencing library prepared using any one of the herein-disclosed methods.

In another aspect, the present disclosure provides a kit for performing any one or more of the herein-disclosed methods.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A-1C. 1A provides a system overview for identifying eccDNA molecules, in accordance with an embodiment. 1B provides a flowchart with steps that can be taken according to embodiments of the present disclosure to identify putative eccDNA molecules. 1C depicts a flowchart including steps for identifying putative eccDNA molecules, in accordance with an embodiment.

FIG. 2. Paired blood and sperm samples from 6 patients were analyzed by duplex sequencing using the TwinStrand DuplexSeq Mutagenesis Assay. All sperm samples had fewer point mutations than matched blood samples, but many more indel calls.

FIGS. 3A-3B. Pooled blood and sperm samples (unmatched) were analyzed by duplex sequencing using the TwinStrand DuplexSeq Mutagenesis Assay, using either mechanical fragmentation (MF) or enzymatic fragmentation (EF). FIG. 3A: Sperm samples had a lower frequency of point mutations and a higher frequency of indel calls, compared to blood. The excess indels in sperm DNA were primarily insertions (not shown) and were detected in libraries prepared with both fragmentation methods, although indel frequency made up a higher proportion of total variant calls in sperm EF libraries (bottom). FIG. 3B: Size distribution of indel calls in sperm DNA shows a periodicity similar to that reported for eccDNA, especially the microDNA subtype.

FIG. 4. Indel calls plotted by indel length for control DNA and sperm DNA libraries, prepared either by mechanical fragmentation (MF) or enzymatic fragmentation (EF). All apparent insertions greater than 20 bp in length were analyzed to determine the source of the inserted DNA. If the called insertion sequence matched the sequence immediately downstream of the called insertion location, creating a BA junction as shown in FIG. 1B, the event was called as an apparent tandem duplication (IsTandemDup=TRUE, orange). If the inserted DNA mapped elsewhere on the same chromosome or nowhere on the same chromosome, events were called as FALSE and NA respectively. Left panel: counts for indel calls plotted by indel length for control DNA (also referred to in the examples as “devDNA”). Right panel: counts for indel calls plotted by indel length for sperm DNA samples. The excess insertions present in sperm cells were almost exclusively apparent tandem duplications containing BA junctions. All insertion calls >100 bp in length were apparent tandem duplications.

FIGS. 5A-5D. Sequence reads and primary alignments for a particular indel call. FIG. 5A: An example variant call with the sequence color-coded to correspond with subsequent panels. FIG. 5B: The two consensus “reads” that support the variant call with different parts of the sequences color-coded to correspond with other panels. The asterisks mark the novel junction and italics indicate the soft-clipped portion of the reads. FIG. 5C: Read alignments shown in IGV, with arrows added in to correspond with the color-coding in other panels. FIG. 5D: Schematic of circular DNA molecule derived from genomic DNA and having a novel junction at the site of circularization (shown as the distinct interface of gray), with scissors indicating a single cleavage event generating a fragment with the exact length as the indel call.

FIG. 6A-6C. 6A is a schematic of (i) a reference allele ABCD, (ii) a duplex consensus read pair, (iii) split alignment of the R1 consensus read supporting a novel junction between the D and A (D-A junction), and two possible alternate alleles containing D-A junctions. Both (iv) excision and circularization of ABCD to form a DNA circle and (v) a chromosomal tandem duplication (TD) of ABCD create a D-A junction. 6B is a histogram of allele length for all insertion calls identified in sperm DNA, colored by whether they contain a D-A junction. The vertical dashed line is at the median fragment size (approximated by median insert size) of the libraries (233 bp). 6C shows a subset of events with allele length smaller than the median fragment size and containing a D-A junction (gray events left of vertical dashed line in B, n=63), (i) chromosomal TDs should have fragment sizes that mirror the distribution of the whole library (grey distributions, respectively), but DNA circles should have fragment sizes less than or equal to the allele size, and thus, less than the median fragment size (diagonal hashes). (ii) All observed fragments were smaller than the median fragment size, significantly different from the expected distribution for chromosomal TDs (binomial p-value=1.08×10-19).

FIG. 7A-7C. Schematic showing the relationship between allele length and fragment size for a D-A junction-containing molecule arising from circular DNA or a chromosomal tandem duplication (TD). For a reference allele ABCD having an allele length of 400 bp (FIG. 7A), if an indel variant maps to that reference and results from a circular DNA (FIG. 7B, left), one fragmentation cleavage event (represented with scissors) will yield a linear fragment with length equal to the allele length of 400 bp (FIG. 7C, left). Two or more cleavages of the circular DNA molecule will yield fragments smaller than the allele length (not shown). In contrast, if the indel variant mapping to that reference results instead from a chromosomal TD of ABCD (FIG. 7B, right), fragmentation of the indel variant can result in fragments smaller than the reference allele length (not shown), equal to size of the allele length (FIG. 7C, left), or longer than the reference allele length of 400 bp (FIG. 7C, right). Note that fragments longer than the allele length (FIG. 7C, right) will include multiple copies of at least some portion of the reference allele.

FIG. 8. Exonuclease V treatment increased the relative frequency (putative circles per duplex base pair) of candidate DNA circles in HeLa and human sperm DNA, as detected by DS. Error bars represent Wilson binomial confidence intervals.

FIG. 9A-9B. 9A shows the frequency of putative DNA circles per duplex base pair detected by DS is higher in tumor DNA, compared to matched normal DNA. Insert numbers indicate the number of unique candidate DNA circles detected in each sample. 9B shows that general somatic mutation frequencies (calculated from the same DS datasets) show no clear correlation to DNA circle frequency, suggesting that the two measurements are independent. In both panels, error bars represent Wilson binomial confidence intervals.

FIG. 10A-10B. 10A shows the frequency of putative DNA circles per duplex base pair, detected by DS, in human TK6 cells treated with different genotoxic compounds. For each of the three compounds, candidate circle frequency shows a dose-responsive increase with at least one dose group being significantly higher than the untreated group. 10B shows mutation frequencies (calculated from the same DS datasets) show no clear correlation to DNA circle frequency, suggesting that the two measurements are independent. In both panels, individual points represent measures from replicate cultures and error bars represent group-based confidence intervals calculated using a t-distribution. Asterisks denote p-values calculated from a quasi-Poisson generalized linear model comparing groups: * p<0.05, ** p<0.01, *** p<0.001.

FIG. 11A-11B. 11A shows the frequency of putative DNA circles per duplex base pair, detected by DS, in human TK6 cells co-cultured with HepaRG cells and treated with water or cyclophosphamide, compared to control. DNA circle frequency is significantly higher in cyclophosphamide-treated samples. 11B shows mutation frequency is significantly higher in cyclophosphamide-treated samples, compared to control. In both panels, individual points represent measures from replicate cultures and error bars represent group-based confidence intervals calculated using a t-distribution. Asterisks denote p-values calculated from a quasi-Poisson generalized linear model comparing groups: * p<0.05, ** p<0.01, *** p<0.001.

### DETAILED DESCRIPTION

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. Where ranges are provided herein, the endpoints are included. As used in this application, the term “comprise” and variations of the term, such as “comprising” and “comprises,” are not intended to exclude other additives, components, integers or steps.

The terms “about” or “approximately,” when used herein in reference to a value, refer to a value that is similar, in context to the referenced value. In general, those skilled in the art, familiar with the context, will appreciate the relevant degree of variance encompassed by “about” or “approximately” in that context. For example, in some embodiments, the term “about” or “approximately” may encompass a range of values that are within 25%, 20%, 19%, 18%, 17%, 16%, 15%, 14%, 13% 12%, 11%, 10%, 9%, 8%, 7%, 6%, 5%, 4%, 3%, 2%, 1%, or less of the referred value.

Unless the word “or” is expressly limited to mean only a single item exclusive from the other items in reference to a list of two or more items, the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the disclosure have been described in the context of those embodiments, other embodiments may also exhibit such advantages, and not all embodiments need necessarily exhibit such advantages to fall within the scope of the disclosure. Accordingly, the disclosure and associated technology can encompass other embodiments not expressly shown or described herein.

The term “subject” encompasses a cell, tissue, or organism, human or non-human, whether in vivo, ex vivo, or in vitro, male or female.

The term “mammal” encompasses both humans and non-humans and includes but is not limited to humans, non-human primates, canines, felines, murines, bovines, equines, and porcines.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour.

It must be noted that, as used in the specification, the singular forms “a,” “an” and “the” include plural referents unless the context clearly dictates otherwise. The definitions and descriptions below in relation to various dependent features may be used with any of the methods described. When described in association with any specific method below, this is for illustrative purposes, to aid explanation, and does not limit the feature to that particular method.

### I. OVERVIEW

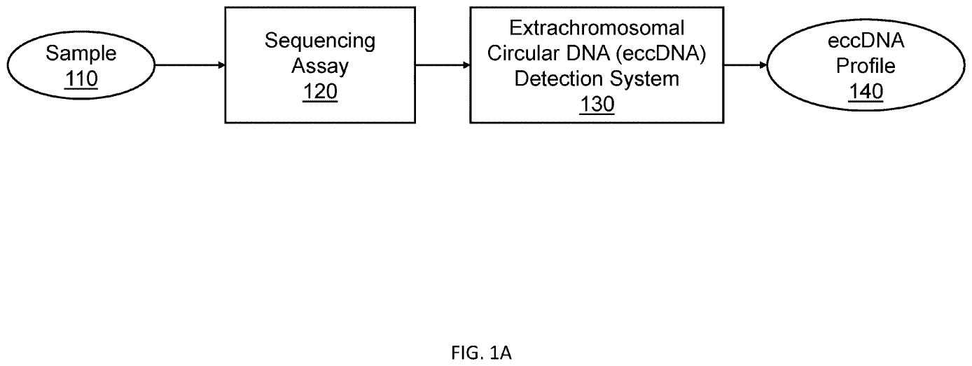

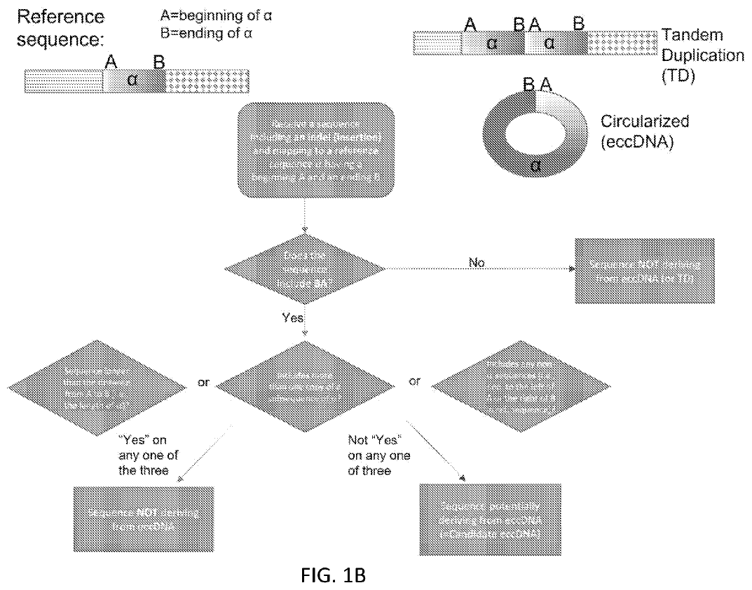

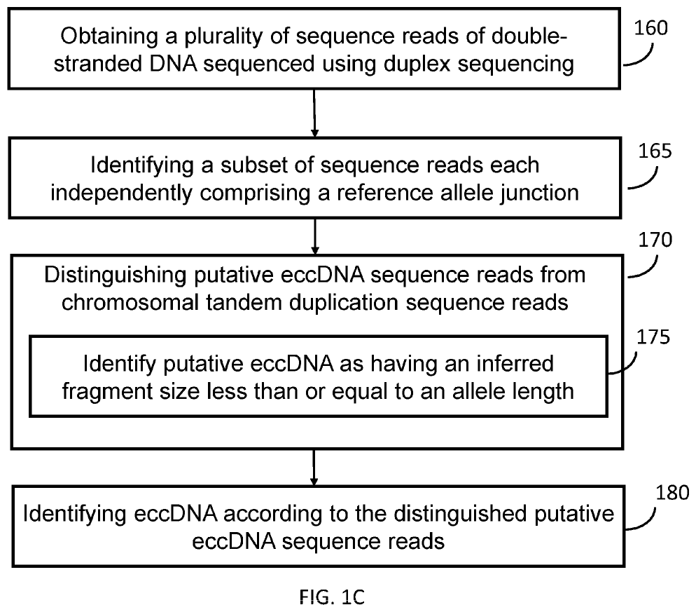

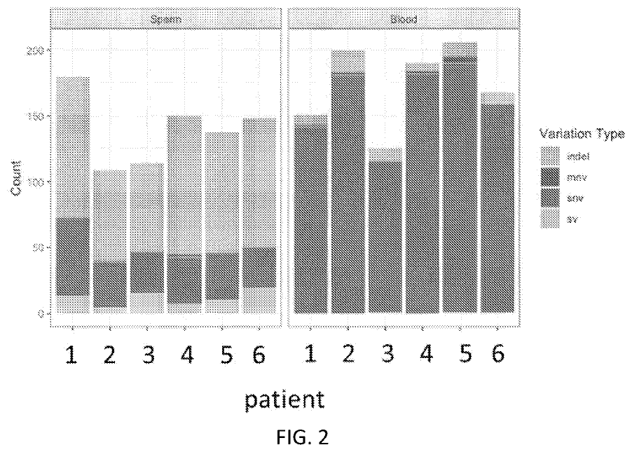

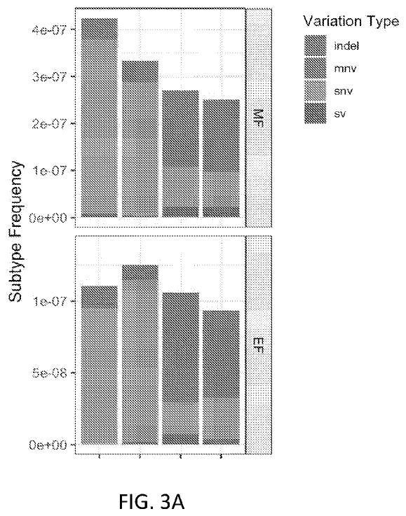

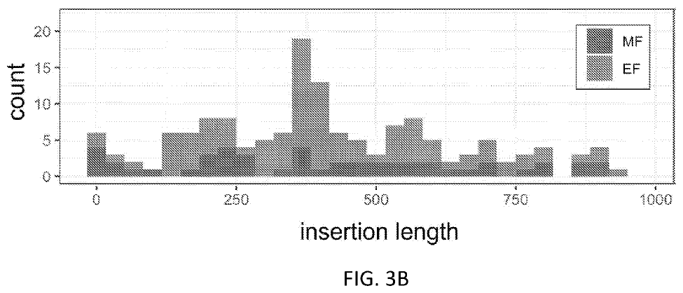

Disclosed herein are methods for identifying extrachromosomal circular DNA (eccDNA) from a sample. Reference is made to FIG. 1A, provides a system overview for identifying eccDNA molecules, in accordance with an embodiment. FIG. 1A introduces a sample 110, a sequencing assay 120, and an extrachromosomal circular DNA (eccDNA) Detection system 130.

In various embodiments, a sample 110 is obtained from a subject. In various embodiments, the sample can be obtained by an individual or by a third party, e.g., a medical professional. Examples of medical professionals include physicians, emergency medical technicians, nurses, first responders, psychologists, phlebotomist, medical physics personnel, nurse practitioners, surgeons, dentists, and any other obvious medical professional as would be known to one skilled in the art. In various embodiments, a sample 110 includes a cell or a population of cells. For example, the cell or population of cells can be previously exposed to a compound or a xenobiotic. In such embodiments, the system overview shown in FIG. 1A is useful for evaluating the compound or xenobiotic provided to the cell or population of cells.

Generally, the sample 110 is analyzed by performing a sequencing assay 120 to determine a plurality of sequence reads of nucleic acids that are present in the sample 110. In particular embodiments, the sequencing assay 120 involves performing an error-corrected sequencing method, an example of involves duplex sequencing (DS). Duplex sequencing is described in further detail herein.

The eccDNA detection system 130 analyzes the plurality of sequence reads generated by the sequencing assay 120 to identify presence or absence of eccDNA. In various embodiments, the eccDNA detection system 130 identifies putative eccDNA sequence reads that include a reference allele junction. The eccDNA detection system 130 can distinguish between eccDNA sequence reads and non-eccDNA sequence reads (e.g., sequence reads derived from chromosomal tandem duplication) and determines an eccDNA profile 140. In various embodiments, the eccDNA profile 140 refers to at least a quantity of eccDNA molecules. In various embodiments, the eccDNA profile 140 refers to at least a frequency of eccDNA molecules.

The eccDNA profile 140 is, in various embodiments, useful for various purposes. For example, the eccDNA profile 140 may be useful for evaluating clastogenicity of a potential clastogen that was previously provided to a cell in the sample 110. As another example, the eccDNA profile 140 may be useful for evaluating genotoxicity of a xenobiotic that was previously provided to a cell in the sample 110. In various embodiments, the eccDNA profile 140 is useful for evaluating cancer risk in the sample 110. For example, a sample with higher quantities or frequencies of eccDNA can be assessed to have a higher risk of cancer in comparison to a different sample with lower quantities or frequencies of eccDNA.

### II. DETECTION OF ECCDNAS IN A BIOLOGICAL SAMPLE

The present disclosure relates to methods and associated reagents, kits, and systems for detecting, quantifying, and characterizing circular DNA molecules in biological samples using error-corrected sequencing methods such as duplex sequencing (DS). Such error-corrected sequencing provides the unexpected advantage of being able to detect small numbers of sequences in a sample deriving from extrachromosomal circular DNA (eccDNA). The present disclosure provides methods for preparing sequencing libraries from any biological sample that may contain eccDNAs, such that eccDNAs present in the sample are maintained and/or enriched. The disclosure also provides methods for sequencing and analyzing libraries prepared from such biological samples that allow the detection, characterization, and/or quantification of eccDNAs. Also provided are methods for identifying additional conditions and features for the preparation and/or analysis of sequencing libraries that can improve the identification of eccDNAs in the sample. The present methods and reagents can be used in connection to any application that involves the preparation of DNA libraries by fragmenting double-stranded DNA molecules and ligating adapters to the resulting double-stranded DNA fragments. The present methods can be used in the preparation of libraries prepared from any source of double-stranded DNA that potentially comprises eccDNAs, such as cell samples, tissue samples, blood samples, biopsies, liquid biopsies, cell-free samples, forensic samples, environmental samples, or other sources.

Extrachromosomal circular DNA (eccDNA) molecules are nuclear, cytosomal, or extracellular extrachromosomal circular DNA of endogenous chromosomal origin and may vary in size from less than 100 bp to several megabases. EccDNAs may include, or may also be referred to as ecDNA, covalently closed circular DNA (typically with reference to a circular viral DNA molecule), microDNA, telomeric circles (a group of eccDNA involved in immortalization of telomerase-negative cancers through alternative lengthening of telomeres), and episomes (autonomously replicated circular DNA typically in reference to bacterial DNA). As used herein, eccDNA generally refers to “simple” eccDNA molecules, i.e., eccDNAs that are formed from the circularization of a single contiguous segment from a genome, as opposed to hybrid or chimeric eccDNAs that may also include segments from elsewhere in the same genome or from any other source. Without wishing to be bound by a particular theory, the term “microDNA” may refer to eccDNA molecules that are fewer than 1,000, 2,000, 3,000, 4,000, 5,000, or more base-pairs (bp). Given the small size, microDNAs do not as commonly carry full-length genes as larger eccDNA molecules but may also carry partial genes or microRNAs.

Small eccDNAs, often called microDNAs, have been identified in many cell and tissue types in diverse eukaryotes, including plants, birds, rodents, and humans. The origin of these small circular DNA molecules is unclear, but most proposed mechanisms involve aberrant repair of DNA breaks. Potential functions of microDNAs are similarly elusive. Despite these unknowns, there is growing interest in microDNAs as biomarkers for an array of disease states.

As used herein, the terms “eccDNA,” “DNA circle,” “circle,” “circular DNA,” and “microDNA” can be used interchangeably and refer to circular DNA molecules.

The methods provided herein may be used to detect candidate microDNAs without exonuclease enrichment, allowing for detection of chromosomal mutations and putative microDNAs simultaneously. Because Duplex Sequencing (DS) uses unique molecular identifiers (UMIs) to tag original DNA molecules prior to amplification, DS allows for more quantitative assessment of microDNAs, relative to each other and to chromosomal DNA, than current methods that involve enrichment and/or rolling circle amplification. microDNA abundance has been shown to increase when cells are exposed to diverse clastogens and pro-apoptotic compounds. Thus, microDNA detection by DS-based assays, could provide a valuable read out of genome instability.

In particular embodiments, the present methods are directed to the sequencing-based detection, quantification, and/or characterization of putative eccDNAs in a biological sample. In particular embodiments of this process, double-stranded DNA in a biological sample is fragmented, and sequencing adapters are ligated to one or both ends of the resulting double-stranded DNA fragments. The DNA fragments are then sequenced and analyzed, and consensus sequences for a plurality of the fragments are obtained. When aligned against a reference genome, certain of the fragments are identified, e.g., as apparent indels (i.e., corresponding to small insertions or deletions), or as a subcategory of apparent indels corresponding to apparent insertions, or as apparent structural variants. In some embodiments, the apparent indel is less than or equal to 1,000 bp in length. In some embodiments, the apparent indel is less than or equal to 900 bp in length. In some embodiments, the apparent indel is less than or equal to 800 bp in length. In some embodiments, the apparent indel is less than or equal to 700 bp in length. In some embodiments, the apparent indel is less than or equal to 600 bp in length. In some embodiments, the apparent indel is less than or equal to 500 bp in length. In some embodiments, the apparent indel is less than or equal to 400 bp in length. In some embodiments, the apparent indel is less than or equal to 300 bp in length. In some embodiments, the apparent indel is greater than or equal to 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, 40 bp, 41 bp, 42 bp, 43 bp, 44 bp, 45 bp, 46 bp, 47 bp, 48 bp, 49 bp, 50 bp, 51 bp, 52 bp, 53 bp, 54 bp, 55 bp, 56 bp, 57 bp, 58 bp, 59 bp, 60 bp, 61 bp, 62 bp, 63 bp, 64 bp, 65 bp, 66 bp, 67 bp, 68 bp, 69 bp, 70 bp, 71 bp, 72 bp, 73 bp, 74 bp, 75 bp, 76 bp, 77 bp, 78 bp, 79 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 98 bp, 99 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, or more in length. In some embodiments, the apparent indel is about 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, 40 bp, 41 bp, 42 bp, 43 bp, 44 bp, 45 bp, 46 bp, 47 bp, 48 bp, 49 bp, 50 bp, 51 bp, 52 bp, 53 bp, 54 bp, 55 bp, 56 bp, 57 bp, 58 bp, 59 bp, 60 bp, 61 bp, 62 bp, 63 bp, 64 bp, 65 bp, 66 bp, 67 bp, 68 bp, 69 bp, 70 bp, 71 bp, 72 bp, 73 bp, 74 bp, 75 bp, 76 bp, 77 bp, 78 bp, 79 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 98 bp, 99 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, or more in length. In some embodiments, the apparent indel at least 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, 40 bp, 41 bp, 42 bp, 43 bp, 44 bp, 45 bp, 46 bp, 47 bp, 48 bp, 49 bp, 50 bp, 51 bp, 52 bp, 53 bp, 54 bp, 55 bp, 56 bp, 57 bp, 58 bp, 59 bp, 60 bp, 61 bp, 62 bp, 63 bp, 64 bp, 65 bp, 66 bp, 67 bp, 68 bp, 69 bp, 70 bp, 71 bp, 72 bp, 73 bp, 74 bp, 75 bp, 76 bp, 77 bp, 78 bp, 79 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 98 bp, 99 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, or more in length. In some embodiments, the apparent indel is 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, 40 bp, 41 bp, 42 bp, 43 bp, 44 bp, 45 bp, 46 bp, 47 bp, 48 bp, 49 bp, 50 bp, 51 bp, 52 bp, 53 bp, 54 bp, 55 bp, 56 bp, 57 bp, 58 bp, 59 bp, 60 bp, 61 bp, 62 bp, 63 bp, 64 bp, 65 bp, 66 bp, 67 bp, 68 bp, 69 bp, 70 bp, 71 bp, 72 bp, 73 bp, 74 bp, 75 bp, 76 bp, 77 bp, 78 bp, 79 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 98 bp, 99 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, or more in length.

In some embodiments, the apparent indel is more than 1, 2, 3, 4, 5, 6, 7, 8, 9, 10, 11, 12, 13, 14, 15, 16, 17, 18, 19, or 20 bp in length. In some embodiments, the apparent indel is more than 20 bp in length.

In some embodiments, the apparent insertion is greater than or equal to 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, 40 bp, 41 bp, 42 bp, 43 bp, 44 bp, 45 bp, 46 bp, 47 bp, 48 bp, 49 bp, 50 bp, 51 bp, 52 bp, 53 bp, 54 bp, 55 bp, 56 bp, 57 bp, 58 bp, 59 bp, 60 bp, 61 bp, 62 bp, 63 bp, 64 bp, 65 bp, 66 bp, 67 bp, 68 bp, 69 bp, 70 bp, 71 bp, 72 bp, 73 bp, 74 bp, 75 bp, 76 bp, 77 bp, 78 bp, 79 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 98 bp, 99 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, or more in length. In some embodiments, the apparent insertion is about 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, 40 bp, 41 bp, 42 bp, 43 bp, 44 bp, 45 bp, 46 bp, 47 bp, 48 bp, 49 bp, 50 bp, 51 bp, 52 bp, 53 bp, 54 bp, 55 bp, 56 bp, 57 bp, 58 bp, 59 bp, 60 bp, 61 bp, 62 bp, 63 bp, 64 bp, 65 bp, 66 bp, 67 bp, 68 bp, 69 bp, 70 bp, 71 bp, 72 bp, 73 bp, 74 bp, 75 bp, 76 bp, 77 bp, 78 bp, 79 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 98 bp, 99 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, or more in length. In some embodiments, the apparent insertion at least 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, 40 bp, 41 bp, 42 bp, 43 bp, 44 bp, 45 bp, 46 bp, 47 bp, 48 bp, 49 bp, 50 bp, 51 bp, 52 bp, 53 bp, 54 bp, 55 bp, 56 bp, 57 bp, 58 bp, 59 bp, 60 bp, 61 bp, 62 bp, 63 bp, 64 bp, 65 bp, 66 bp, 67 bp, 68 bp, 69 bp, 70 bp, 71 bp, 72 bp, 73 bp, 74 bp, 75 bp, 76 bp, 77 bp, 78 bp, 79 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 98 bp, 99 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, or more in length. In some embodiments, the apparent insertion is 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, 40 bp, 41 bp, 42 bp, 43 bp, 44 bp, 45 bp, 46 bp, 47 bp, 48 bp, 49 bp, 50 bp, 51 bp, 52 bp, 53 bp, 54 bp, 55 bp, 56 bp, 57 bp, 58 bp, 59 bp, 60 bp, 61 bp, 62 bp, 63 bp, 64 bp, 65 bp, 66 bp, 67 bp, 68 bp, 69 bp, 70 bp, 71 bp, 72 bp, 73 bp, 74 bp, 75 bp, 76 bp, 77 bp, 78 bp, 79 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 98 bp, 99 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, or more in length.

In some embodiments, the apparent insertion is more than 1, 2, 3, 4, 5, 6, 7, 8, 9, 10, 11, 12, 13, 14, 15, 16, 17, 18, 19, or 20 bp in length. In some embodiments, the apparent insertion is more than 20 bp in length.

In some embodiments, the apparent structural variant is due to an apparent insertion, or an apparent duplication.

Once apparent indels and/or apparent structural variants have been identified, the sequences can be assessed to determine whether they are derived from putative eccDNA molecules, and in some embodiments a probability is calculated or estimated that a given putative eccDNA molecule is an eccDNA molecule. FIG. 1B, for example, includes a flowchart with steps that can be taken, according to embodiments of the present disclosure, to identify putative eccDNAs or putative eccDNAs in a sample. In the first step of the flowchart as shown in FIG. 1B, these indels or insertions are identified or provided. This category of insertion sequences can include, e.g., tandem duplications (TDs), insertions that are not tandem duplications (non-TD insertions), and eccDNA molecules. In the next step in the flowchart as shown in FIG. 1B, non-TD insertions are eliminated from the category by detecting putative eccDNA breakpoint sequences, represented in FIG. 1B as “BA” breakpoints. Such breakpoints, which can potentially be present in eccDNAs as well as tandem duplications, include a sequence A and a sequence B that are both present on the same chromosome in the reference genome, separated by a distance of Y nucleotides. For example, the distance Y can start at the first nucleotide of sequence A and continue to the last nucleotide of sequence B (as shown in FIG. 1B, where a refers the entire sequence running from the first nucleotide of A to the last nucleotide of B, such that the length of a in the genome is equal to Y nucleotides).

In contrast to the arrangement of A and B in the reference genome, at a candidate breakpoint the A and B sequences are approximately immediately adjacent to one another, and their order is reversed on the chromosome. FIG. 1B illustrates how such a sequence starting with A (i.e., the first nucleotide of A) and terminating with B (i.e., the last nucleotide of B) in the reference genome could, through either a tandem duplication event or by the formation of an eccDNA, result in the creation of the candidate breakpoint BA sequence. In some embodiments, the breakpoint includes the last nucleotide of sequence B immediately upstream of the first nucleotide of sequence A. It will be appreciated, however, that due to potential imprecision in the closing of an eccDNA circle, in some embodiments the final nucleotide of B will be separated from the first nucleotide of A by 1 or more nucleotides, and/or in some embodiments the final nucleotide of B and/or the first nucleotide of A may be absent or mutated.

Next, as illustrated in FIG. 1B, the sequences that contain a putative eccDNA breakpoint are assessed in any one or more of various ways to assess the likelihood that they correspond to true eccDNA molecules. These assessments are based upon several properties of eccDNAs (i.e., “simple” eccDNAs that are formed from the circularization of a single contiguous segment from a genome, as opposed to hybrid or chimeric eccDNAs that may also include segments from elsewhere in the same genome or from any other source) that allow them to be distinguished from tandem duplications. For example, because a simple eccDNA formed as shown in FIG. 1B will consist essentially of the sequence a, running from the first nucleotide of sequence A to the last nucleotide of sequence B (and having the length Y), any sequence derived from a genuine eccDNA will not be longer than the distance Y, it will not contain sequences shown to the left of A or to the right of B in FIG. 1B, and it will not include more than one copy of any part of a.

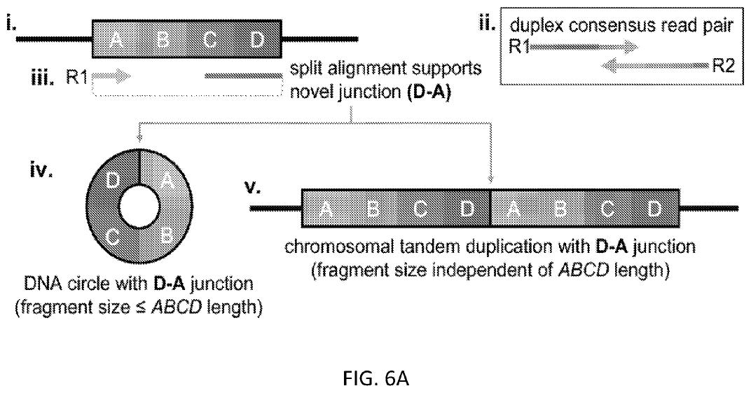

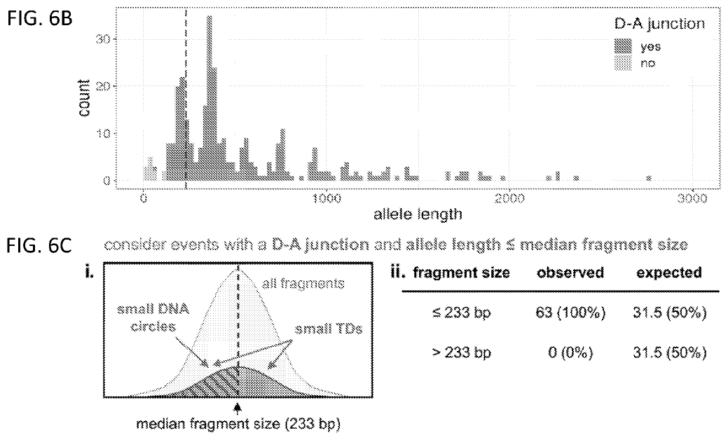

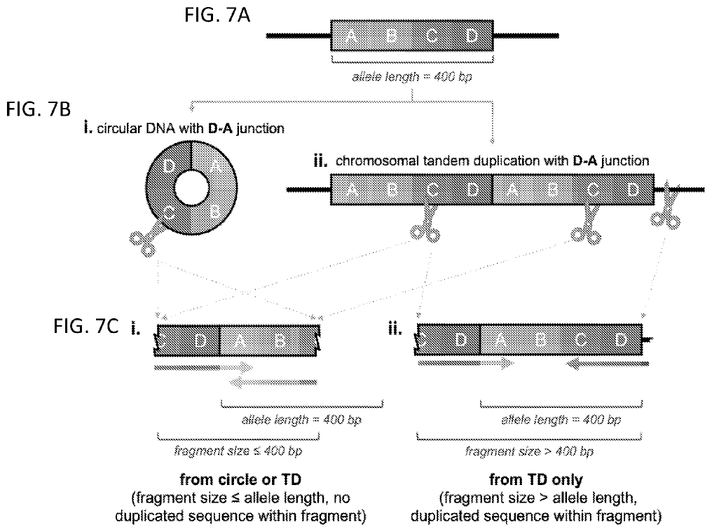

Accordingly, any determination that a sequence with a BA breakpoint is longer than Y (e.g., contains 10, 20, 30, 40, 50, 60, 70, 80, 90, 100, or more nucleotides than Y), that it contains any sequences shown to the left of A or to the right of B in FIG. 1B, FIG. 6A, or FIG. 7A-7C (e.g., any stretch of, e.g., 5, 6, 7, 8, 9, 10, 15, 20, 25, 30, nucleotides or more to left of A or to the right of B), or that it includes more than one copy of subsequence within a (e.g., any stretch of, e.g., 5, 6, 7, 8, 9, 10, 15, 20, 25, 30, nucleotides or more of a), indicates that the sequence is not derived from an eccDNA. It will be appreciated that it is not necessary to perform all three of these assessments, as any of the three would be sufficient. Accordingly, in some embodiments only a single assessment out of the three is used.

In some embodiments, the length of the fragment comprising the putative eccDNA (i.e., or the length of a consensus sequence derived from the fragment) is also compared to the distance Y to determine if the length of the fragment or consensus sequence is approximately or exactly equal to Y. As noted above, because an eccDNA formed as shown in FIG. 1B, FIG. 6A, or FIG. 7A-7C will have the same approximate length as distance Y, a fragment in a sequencing library that is derived from the eccDNA will also have the approximate length Y if the fragment has only been cut (i.e., linearized) one time during the preparation of the library. Alternatively, if an eccDNA is cut more than once during fragmentation, and a resulting fragment comprising the BA sequence is ligated to sequencing adapters and included in the library, then the inserted fragment will be shorter than the length Y.

Accordingly, an observation that the length of a fragment or consensus sequence equals, or approximately equals, distance Y, can provide evidence that the fragment is indeed derived from a genuine eccDNA (as opposed to from a tandem duplication where the length of the fragment is by chance equal to Y, which would typically be less likely to occur, especially when the average length of fragments in the library is significantly greater than the distance Y). Such evidence can be used, e.g., in a calculation or estimation of the probability that a given fragment or consensus sequence is indeed derived from a genuine eccDNA.

Reference is further made to FIG. 1C, which depicts a flowchart including steps for identifying putative eccDNA molecules, in accordance with an embodiment. In particular embodiments, the identifying at least one eccDNA in a sample is identifying presence or absence of eccDNA in the sample.

Step 160 involves obtaining a plurality of sequence reads of double-stranded DNA sequenced using duplex sequencing. In some embodiments, obtaining a plurality of sequence reads of double-stranded DNA sequenced using duplex sequencing comprises performing or having performed duplex sequencing on the sample. In some embodiments, the duplex sequencing comprises ligating adaptors to the ends of the double-stranded DNA, wherein at least one adaptor comprises a nucleotide sequence that tags a strand of the double-stranded DNA such that the strand of the double-stranded DNA has a distinctly identifiable nucleotide sequence relative to its complementary strand. In some embodiments, the duplex sequencing comprises amplifying strands of the double-stranded DNA using the ligated adaptors to generate at least first strand amplicons and second strand amplicons. In some embodiments, the duplex sequencing comprises sequencing at least the first strand amplicons and the second strand amplicons to produce a plurality of sequence reads comprising first strand sequence reads and second strand sequence reads. In some embodiments, obtaining a plurality of sequence reads of double-stranded DNA sequenced using duplex sequencing comprises identifying or having identified the eccDNA using the plurality of sequence reads of the duplex sequencing.

Step 165 involves identifying a subset of sequence reads each independently comprising a reference allele junction. The reference allele junction (e.g., D-A) is a junction comprising the nucleic acid sequence of the end of the reference allele (e.g., D for reference allele ABCD) conjugated to the nucleic acid sequence of the beginning of the reference allele (e.g., A for reference allele ABCD).

Step 170 involves distinguishing putative eccDNA sequence reads from chromosomal tandem duplication sequence reads. In various embodiments, further to distinguishing putative eccDNA sequence reads from chromosomal tandem duplications sequence reads, step 175 involves selecting a subset of D-A junction-containing consensus sequencing reads with allele length less than a threshold apparent insert size, inferring the fragment size of each read pair in the subset and comparing it to the threshold apparent insert size, one by one, or comparing the distribution of inferred fragment sizes to the distribution of insert sizes in the entire library, and comparing the inferred fragment size of any one consensus read pair to the allele size of that read pair.

Step 180 involves identifying eccDNA according to the distinguished putative eccDNA sequence reads of step 170. In some embodiments, identifying eccDNA according to the distinguished putative eccDNA sequence reads comprises determining a quantity of eccDNA according to the distinguished putative eccDNA sequence reads. In some embodiments, identifying eccDNA according to the distinguished putative eccDNA sequence reads comprises determining a frequency of eccDNA according to the distinguished putative eccDNA sequence reads. In some embodiments, identifying eccDNA according to the distinguished putative eccDNA sequence reads comprises determining a quality of eccDNA according to the distinguished putative eccDNA sequence reads. In some embodiments, identifying eccDNA according to the distinguished putative eccDNA sequence reads comprises determining any characteristic of eccDNA according to the distinguished putative eccDNA sequence reads. In some embodiments, the any characteristic of eccDNA may include, but is not limited to, size, location of chromosomal origination, annotation of chromosomal origination (genic, exotnic, intronic, regulatory elements, repetitive elements, etc), and/or nucleotide sequence content (GC content, mono-, di-, tri-nucleotide repeats, microhomology at junction, etc).

Accordingly, in some embodiments a “candidate eccDNA molecule” or a “putative eccDNA”, may be used interchangeably, and correspond to any double-stranded fragment, or a consensus sequence derived from the fragment, obtained from a biological sample that is identified as an apparent indel or apparent structural variant and that comprises a putative eccDNA breakpoint (i.e., a “BA” or “DA”, which may be used interchangeably, breakpoint in FIG. 1B, FIG. 6A, or FIG. 7A-7C). In some embodiments, a putative eccDNA is not shown: to be (i) longer than distance Y, (ii) to comprise any sequences located outside of the genomic region delineated by A and B, or A, B, C, and D in the reference genome (i.e., to the left of A or right of B in FIG. 1B, or to the left of A and to the right of D in FIG. 6A, or FIG. 7A-7C), and (iii) to comprise more than one copy of any subsequence from the region a starting at the first nucleotide of sequence A and continuing to the last nucleotide of sequence B in the reference genome, is considered to correspond to a putative eccDNA molecule.

As noted above, in some embodiments such “putative eccDNA” may include, in addition to true eccDNA molecules, short fragments (i.e., shorter than distance Y) derived from a tandem duplication. Accordingly, the present disclosure provides additional steps that can be taken to help identify genuine eccDNA molecules among candidates in this category, and/or that can help estimate or calculate the probability that a given candidate is indeed an eccDNA molecule.

In some embodiments, information concerning the size of the fragment corresponding to the putative eccDNA molecule relative to the size of fragments in the sequencing library can be used to inform a calculation or estimation regarding the probability that the fragment is indeed derived from a genuine eccDNA molecule. For example, as noted elsewhere herein, an observation that the fragment has the exact or approximate size of distance Y can indicate an increased likelihood that the fragment is indeed derived from a genuine eccDNA molecule, in particular when the average length of fragments in the library is greater than Y.

Further, the more the average length of fragments in the library exceeds Y, and the less variation there is in the size of the fragments (e.g., the lower the standard deviation from the mean fragment length), the greater the probability that the fragment is a true eccDNA and not a small fragment of a tandem duplication occurring by chance. In some embodiments of the present methods, as described elsewhere herein, libraries are prepared with relatively high average fragment lengths in order to lower the likelihood that a given fragment in the library will have a size equal to or lower than Y nucleotides by chance. In some embodiments, the average (e.g., mean or median) length of fragments in the library is between about 100 bp and 1000 bp. In some embodiments, the average (e.g., mean or median) length of fragments in the library is about 1000 bp, 2000 bp, 3000 bp, 4000 bp, 5000 bp, 6000 bp, 7000 bp, 8000 bp, 9000 bp, or 10,000 bp.

In some embodiments, the length of the putative eccDNA molecule is between about 100 and 1000 nucleotides, or is less than about 500 nucleotides. In some embodiments, the length of the putative eccDNA molecule is greater than about 1000 bp, 2000 bp, 3000 bp, 4000 bp, 5000 bp, 6000 bp, 7000 bp, 8000 bp, 9000 bp, or 10,000 bp. In some embodiments, the length of the putative eccDNA molecule is greater than about 100 kb, 200 kb, 300 kb, 400 kb, 500 kb, 600 kb, 700 kb, 800 kb, 900 kb, 1 Mb, 2 Mb, or 3 Mb.

In some embodiments, the putative eccDNA molecule comprises a gene (e.g., a coding sequence, promoter, regulatory elements). In some embodiments, the putative eccDNA molecule comprises an origin of replication. In some such embodiments, multiple copies of the eccDNA is expected or observed to be present in one or more cells of the sample.

In particular embodiments, the present methods are performed multiple times using a given biological sample, with one (or more) performed with an enrichment step for circular DNA molecules, and one (or more) time performed without such an enrichment step. An enrichment step for eccDNA molecules can be performed in any of a number of ways. For example, in some embodiments, linear (i.e., non-circular) DNA molecules are removed from the sample, e.g., using one or more exonucleases. In such embodiments, some or all of the linear DNA in the sample is digested, with only the circular DNA molecules in the sample remaining. In some embodiments, circular DNA molecules are selectively isolated or purified from a sample. As long as a method can separate linear double-stranded DNA molecules from covalently closed circular double-stranded DNA molecules, it can be used in the present methods.

By performing the methods with and without an enrichment step and comparing the putative eccDNA molecules detected in the two cases, it is possible to determine or estimate how many genuine eccDNA molecules were present in the original sample, i.e., to determine or estimate what proportion of the putative eccDNA molecules detected in the absence of exonuclease treatment are genuine eccDNA molecules.

A non-limiting list of exonucleases that can be used in such methods includes, e.g., exonuclease I, exonuclease T, exonuclease VII, exonuclease III, T7 exonuclease, exonuclease V(RecBCD), exonuclease VIII, lambda exonuclease, and/or T5 exonuclease. In some embodiments, one or more exonucleases are used. In some embodiments, the one or more exonucleases comprise exonuclease I, exonuclease T, exonuclease VII, exonuclease III, T7 exonuclease, exonuclease V (RecBCD), exonuclease VIII, lambda exonuclease, or T5 exonuclease. In some embodiments, the one or more exonucleases comprise exonuclease I. In some embodiments, the one or more exonucleases comprise exonuclease T. In some embodiments, the one or more exonucleases comprise exonuclease VII. In some embodiments, the one or more exonucleases comprise exonuclease III. In some embodiments, the one or more exonucleases comprise T7 exonuclease. In some embodiments, the one or more exonucleases comprise exonuclease V (RecBCD). In some embodiments, the one or more exonucleases comprise exonuclease VIII. In some embodiments, the one or more exonucleases comprise lambda exonuclease. In some embodiments, the one or more exonucleases comprise T5 exonuclease.

In some embodiments, a portion of the sample will also be treated with an endonuclease prior to exonuclease treatment. The endonucleases can linearize circular DNA molecules in the sample and cause the removal of all double-stranded DNA molecules in the sample, providing further evidence that any DNA molecules remaining after exonuclease treatment are indeed circular DNA molecules.

In some embodiments, the enrichment step comprises performing a size selection. In some embodiments, the size selection comprises a use of paramagnetic beads, electrophoresis, column filtrations, density gradient centrifugation, or selective extraction. In some embodiments, the size selections is conducted at a size threshold of about 10,000 bp. In some embodiments, the size selection comprises a use of paramagnetic beads, electrophoresis, column filtrations, density gradient centrifugation, or selective extraction, at a size threshold of at least 10,000 bp. In some embodiments, the size selection uses a size threshold of about 10,000 bp. In some embodiments, the size selection uses a size threshold of at least 10,000 bp. In some embodiments, the size selection comprises a use of paramagnetic beads. In some embodiments, the size selection comprises a use of electrophoresis. In some embodiments, the size selection comprises a use of column filtration. In some embodiments, the size selection comprises a use of density gradient centrifugation. In some embodiments, the size selection comprises a use of selective extraction. In some embodiments, the size selection comprises a use of paramagnetic beads at a size threshold of about 10,000 bp. In some embodiments, the size selection comprises a use of electrophoresis at a size threshold of about 10,000 bp. In some embodiments, the size selection comprises a use of column filtration at a size threshold of about 10,000 bp. In some embodiments, the size selection comprises a use of density gradient centrifugation at a size threshold of about 10,000 bp. In some embodiments, the size selection comprises a use of paramagnetic beads at a size threshold of at least 10,000 bp. In some embodiments, the size selection comprises a use of electrophoresis at a size threshold of at least 10,000 bp. In some embodiments, the size selection comprises a use of column filtration at a size threshold of at least 10,000 bp. In some embodiments, the size selection comprises a use of density gradient centrifugation at a size threshold of at least 10,000 bp. In some embodiments, the size selection comprises a use of selective extraction at a size threshold of at least 10,000 bp.

In some embodiments, the enrichment step comprises electrophoresis, column filtration (e.g., using a silica column designed or used for plasmid isolation), density gradient centrifugation, selective extraction, and/or using a DNA binding protein that will differentially bind to or remain bound to double-stranded circular DNA molecules as compared to double-stranded linear DNA molecules. In some embodiments, the DNA binding protein is a helicase, or another protein that will bind to and translocate along double-stranded DNA, and which will therefore fall off of the ends of linear DNA but not circular DNA, which lacks ends. According to such embodiments, circular DNA molecules could be isolated by binding such proteins to the DNA in a sample, and purifying protein-DNA complexes using, e.g., affinity-based methods (e.g., using antibodies specific to the protein, by biotinylating the protein, or other methods known in the art).

In some embodiments, e.g., where it is critical to be certain that any individual eccDNAs detected is an eccDNA, an enrichment step, such as size selection or an exonuclease treatment, can be systematically performed to ensure that all detected eccDNAs are eccDNAs and not, e.g., fragments of tandem duplications. However, in some embodiments, it will be sufficient to be able to calculate or estimate the likelihood that a given eccDNA molecule is an eccDNA, e.g., based on information obtained during a separate performance of the method that included an enrichment step. Further, it will be appreciated that in cases where an enrichment step is performed, that the performance of the method including the enrichment step does not have to be performed at the same time as a performance of the method without the step. For example, upon collection of a biological sample, an initial analysis may be performed using enrichment, and then one or more subsequent analyses may be performed without enrichment from the same biological sample, and can be performed at any time, including, e.g., months or years after the initial performance of the method including the enrichment step. Similarly, an analysis using an enrichment step may be performed after a first performance of the method without enrichment, e.g., in a scenario where putative eccDNA molecules are detected in a sample and it is desired to repeat the method with an exonuclease or other enrichment step to confirm, quantify, and/or characterize the detected putative eccDNA molecules. Further, in some embodiments, an enrichment step is never performed on a given biological sample, e.g., if sufficient analyses including an enrichment step have been performed on similar or analogous samples in the past to permit the reliable analysis of new samples without enrichment.

In some embodiments, by altering one or more conditions under which a library is prepared and subsequently comparing the candidate and/or the eccDNA molecules detected before or after the alteration, it is possible to identify conditions that allow for, e.g., a higher yield of total eccDNA molecules in a sample and/or a higher yield of any particular type of eccDNA molecules (e.g., eccDNAs of a particular size, with or without a replication origin, with or without a genic region, etc.). Any condition of the library preparation can be altered in such assays, including, but not limited to, steps involved in the isolation of nucleic acids from the sample, cleaning or pre-conditioning of nucleic acids (e.g., DTT treatment), fragmentation conditions (mechanical fragmentation, e.g., sonication, Covaris, enzymatic fragmentation, including the nature and concentration of enzymes used for fragmentation), the duration of a fragmentation step, the types and concentrations of sequencing adapters used, the conditions of the ligation step, the amount of DNA used, and others.

The inclusion of an enrichment step such as a size selection, or an exonuclease treatment will affect different nucleic acids in the biological sample differently. For example, as exonucleases act on nucleic acid ends, they are expected to digest linear nucleic acids in the sample but not circular nucleic acids (in particular, undamaged or un-nicked circular nucleic acids). Accordingly, because the different steps illustrated in the flowchart of FIG. 1B or FIG. 1C may detect various types of linear DNA (e.g., tandem duplications (TDs) and non-TD insertions), the numbers of fragments detected in the different steps are expected to decrease with enrichment for circular DNA molecules. For example, concerning the fragments provided or received in the first step that include an indel or insertion: in the absence of enrichment this category may include TDs, non-TD insertions (or other indels), and eccDNAs, whereas with enrichment the same category may only include eccDNAs (provided that the TDs and non-TD insertions have been completely eliminated by the enrichment step). Similarly, concerning the fragments detected in the subsequent step with a BA breakpoint: in the absence of enrichment this category may include TDs and eccDNAs, whereas with enrichment only eccDNAs will be present. Further, concerning the “putative eccDNA” molecules remaining after the third step, in the absence of enrichment this category can in principle contain both eccDNAs and TD-derived fragments that are shorter than Y, whereas with enrichment only eccDNAs should remain. Accordingly, if non-TD insertions and/or TDs are present among the fragments along with eccDNAs, it is possible that the number of fragments detected in the first two steps will decrease more with enrichment than will the number of putative eccDNAs detected in the final step.

Accordingly, in some embodiments, the ratio of the number of putative eccDNA molecules detected in the final step as shown in FIG. 1B to the total number of error-corrected sequences obtained from the library, or to the number of possible insertions or indels detected among the sequences, or to the number of putative eccDNA breakpoints detected among the insertion- or indel-containing sequences, is higher when the method is performed with the enrichment step than without the enrichment step.

In some embodiments, the number of error-corrected sequences obtained with the enrichment step is less than about 90%, 80%, 70%, 60%, 50%, 40%, 30%, 20%, or 10% of the number of error-corrected sequences obtained without the enrichment step.

In some embodiments, the frequency of an apparent indel or apparent structural variant detected among the error-corrected sequences with the enrichment step is less than about 90%, 80%, 70%, 60%, 50%, 40%, 30%, 20%, or 10% of the frequency of possible insertions or indels detected without the enrichment step.

In some embodiments, the frequency of putative eccDNA breakpoints detected among the fragments with a detected apparent indel or apparent structural variant with the enrichment step is less than about 90%, 80%, 70%, 60%, 50%, 40%, 30%, 20%, or 10% of the frequency of putative eccDNA breakpoints detected without the enrichment step.

In some embodiments, the frequency of putative eccDNA molecules detected among the fragments with a detected eccDNA breakpoint with the enrichment step is at least about 10%, 20%, 30%, 40%, 50%, 60%, 70%, 80%, 90%, or more of the frequency of putative eccDNA molecules detected without the enrichment step.

In some embodiments, e.g., where essentially all of the insertions present in a given sample correspond to eccDNA molecules, the frequencies of the different categories may not significantly decrease with enrichment of circular DNA molecules. In some embodiments, e.g., where essentially all of the insertions present in a given sample correspond to eccDNA molecules, the frequencies of the different categories may increase with enrichment of circular DNA molecules.

In some embodiments, any one or more quantitative element of any of the information obtained in the present methods, such as any of the herein-described frequencies, ratios, or percentages, is used in a calculation to determine or estimate the likelihood that a putative eccDNA molecule identified in step (e) is a genuine eccDNA molecule. In some embodiments, the calculation is performed partially or entirely on a computer and/or in the cloud.

In some embodiments, a calculation in based in part on a relationship between the length of the putative eccDNA molecule to the average length of double-stranded DNA fragments in the library, wherein a lower length of the putative eccDNA molecule relative to the average length of double-stranded DNA fragments in the library indicates a higher probability that the putative eccDNA molecule is a genuine eccDNA molecule.

In some embodiments, a calculation is based in part on an observation that the length of the putative eccDNA molecule is approximately or exactly equal to the distance Y nucleotides, wherein the observation indicates a higher probability that the putative eccDNA molecule is a genuine eccDNA molecule.

Computer-based systems for performing any one or more of the herein-disclosed methods are also provided.

In some embodiments, the present disclosure provides treating a disease or other medical condition in a mammalian subject. In some such embodiments, the method comprises: (i) performing any of the herein-disclosed methods on a biological sample obtained from the subject; (ii) identifying one or more putative eccDNA molecules in the sample that are indicative of the disease or of a physiological state associated with the medical condition; and (iii) treating the subject for the disease or medical condition.

In some embodiments, the disease state or physiological state is selected from the group consisting of cancer, inflammation, autoimmunity, infection, organ transplant rejection, stem cell transplant rejection, therapeutic cell rejection, therapeutic cell response, immunotherapy response, pregnancy, pre-eclampsia, radiation exposure, sun exposure, drug exposure, and hypersensitivity.

### III. PREPARATION OF SEQUENCING LIBRARIES

The present methods can be used for the detection and/or quantification of eccDNAs in a biological sample, based on the analysis of sequence information obtained from double-stranded nucleic acids obtained from biological samples.

### Biological Samples

The present methods can be used for the detection, quantification, and/or characterization of candidate extrachromosomal circular DNA (eccDNA) molecules in any type of biological sample. In some embodiments, the biological sample comprises cells. In some embodiments, the biological sample comprises cell-free DNA. In some embodiments, the biological sample comprises cells and cell-free DNA. In some embodiments, the biological sample is obtained from a subject, e.g., a blood sample, tissue sample, tumor biopsy, liquid biopsy, swab, lavage, urine sample, saliva sample, or any other sample that comprises cells and/or cell-free DNA that can be analyzed using the present methods. In some embodiments, the biological sample comprises sperm cells, e.g., a sperm or semen sample. In some embodiments, the sample is a prostatic fluid sample, testicular biopsy sample, spermatogonia sample, germ cell sample, gamete sample, swab, lavage, aspirate, biopsy, tissue sample, tumor sample, preneoplastic sample, liquid biopsy, hyperplasia sample, hypertrophy sample, dysplastic sample, urine sample, CSF sample, any other body fluid sample, autopsy sample, necropsy sample, surgical sample, model organism sample, plasma sample, serum sample, gastric sample, bone marrow sample, stool sample, brushing sample, bile sample, pancreatic fluid sample, synovial fluid sample, sputum sample, mucus sample, vitreous sample, forensic sample, environmental sample, bacterial sample, fungal sample, mammalian sample, human sample, and diagnostic sample.

In some embodiments, the biological sample comprises cancer cells or nucleic acids derived from cancer cells, e.g., a tumor sample, blood sample, or other liquid biopsy. In some embodiments, the presence and/or character of eccDNA molecules in the sample can indicate the presence of certain genetic events in an individual, e.g., genomic instability related to cancer, apoptotic degradation of DNA, etc. In some embodiments, the presence and/or character of eccDNA molecules in the sample can be used to identify a disease state or a physiological state, e.g., inflammation, autoimmunity, infection, organ transplant rejection, stem cell transplant rejection, therapeutic cell rejection, therapeutic cell response, immunotherapy response, pregnancy, pre-eclampsia, radiation exposure, sun exposure, drug exposure, and hypersensitivity.

In some embodiments, the biological sample comprises cells that have been exposed to a potentially toxic agent, e.g., a potentially clastogenic, aneugenic, mutagenic, and/or teratogenic agent. In some embodiments, the biological sample has been taken from an individual that has been exposed to the agent, or comprises cells that have been deliberately exposed to an agent to assess the genotoxic potential of the agent.

As used herein, the term “biological sample” or “sample” typically refers to a sample obtained or derived from a biological source (e.g., a tissue or organism or cell culture) of interest, as described herein. In some embodiments, a source of interest comprises an organism, such as an animal or human. In other embodiments, a source of interest comprises a microorganism, such as a bacterium, virus, protozoan, or fungus. In further embodiments, a source of interest may be a synthetic tissue, organism, cell culture, nucleic acid or other material. In yet further embodiments, a source of interest may be a plant-based organism. In yet another embodiment, a sample may be an environmental sample such as, for example, a water sample, soil sample, archeological sample, or other sample collected from a non-living source. In other embodiments, a sample may be a multi-organism sample (e.g., a mixed organism sample). In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; pap smear, oral swabs; nasal swabs; washings or lavages such as a ductal lavages or bronchoalveolar lavages; vaginal fluid, aspirates; scrapings; bone marrow specimens; tissue biopsy specimens; fetal tissue or fluids; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In a particular embodiment, a biological sample is a liquid biopsy obtained from a subject. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

In some embodiments, the sample is a forensic sample, e.g., a blood, tissue, sperm, hair saliva, or other sample comprising cells or cell-free DNA from a known or unknown source, and wherein the present methods can be used to identify, e.g., the individual that was the source of the sample and/or the tissue or type of cell from which cell-free DNA originated.

As used herein, the term “subject” refers to an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

### Fragmentation

The double-stranded DNA obtained from the biological sample can be fragmented in any of a number of ways. For example, fragmentation can be achieved by physical shearing (e.g., sonication, Covaris fragmentation) or enzymatic approaches that utilize an enzyme cocktail to cleave DNA phosphodiester bonds. The result of either of the above methods is a sample where the intact nucleic acid material (e.g., genomic DNA (gDNA)) is reduced to a mixture of randomly or semi-randomly sized nucleic acid fragments. In particular embodiments of the present methods, enzymatic fragmentation is used.

### Ligation of Adapters

In particular embodiments, the present methods involve the ligation of one or more sequencing adapters to fragmented double-stranded nucleic acid molecules to produce double-stranded adapter-fragment complexes. Such adapter molecules may include one or more of a variety of features suitable for MPS or Next Generation Sequencing (NGS) platforms such as, for example, sequencing primer recognition sites, amplification primer recognition sites, barcodes (e.g., single molecule identifier (SMI) sequences, indexing sequences, single-stranded portions, double-stranded portions, strand distinguishing elements (SDEs) or features, and the like. The use of highly pure sequencing adapters for DS, or any next-generation sequencing technology, is important for obtaining reproducible data of high quality and maximizing sequence yield of a sample (i.e., the relative percentage of inputted molecules that are converted to independent sequence reads). It is particularly important with DS because of the need to successfully recover both strands of the original duplex molecules.

In some embodiments, the adapters have a Y shape. In some embodiments, the adapters have a loop or a hairpin shape. In some embodiments, one or more of the adapters disclosed in, e.g., U.S. Pat. No. 11,332,784, 11,479,807, 10,287,631, 9,752,188, 11,155,869, 11,098,359, 11,242,562, 11,198,907, 10,570,451, 10,385,393, 10,370,713, 11,130,996, 10,689,699, 10,604,804, 10,689,700, 10,711,304, 10,760,127, 10,752,951, 11,047,006, 11,118,225, 11,608,529, 11,555,220, or U.S. Pat. No. 11,549,144, each of which is hereby incorporated by reference in its entirety, is used.

### IV. SEQUENCING AND ANALYSIS

As described herein, methods involve performing a sequencing assay (e.g., sequencing assay 120 described in reference to FIG. 1A) to analyze a sample. In particular embodiments, sequencing assay involves an error-corrected sequencing method such as duplex sequencing (DS). Duplex Sequencing (DS) is a method for producing error-corrected nucleic acid sequence reads from double-stranded nucleic acid molecules. In certain aspects of the technology, DS can be used to independently sequence both strands of individual nucleic acid molecules in such a way that the derivative sequence reads can be recognized as having originated from the same double-stranded nucleic acid parent molecule during massively parallel sequencing, but also differentiated from each other as distinguishable entities following sequencing. The resulting sequence reads from each strand are then compared for the purpose of obtaining an error-corrected sequence of the original double-stranded nucleic acid molecule, known as a Duplex Consensus Sequence. The process of DS makes it possible to confirm whether one or both strands of an original double-stranded nucleic acid molecule are represented in the generated sequencing data used to form a Duplex Consensus Sequence. Methods of duplex sequencing are disclosed, e.g., in U.S. Pat. Nos. 9,752,188, 11,479,807, 10,287,631, 9,752,188, 11,155,869, 11,098,359, 11,242,562, 11,198,907, 10,570,451, 10,385,393, 10,370,713, 11,130,996, 10,689,699, 10,604,804, 10,689,700, 10,711,304, 10,760,127, 10,752,951, 11,047,006, 11,118,225, 11,608,529, 11,555,220, and 11,549,144, each of which is hereby incorporated by reference in its entirety.

In addition to duplex sequencing, any sequencing modalities capable of generating error-corrected sequencing reads are encompassed by the scope of the present disclosure. For example, many embodiments of single consensus sequencing and/or combinations of single and duplex consensus sequencing are contemplated. Additionally, other embodiments of the present technology can have different configurations, components, or procedures than those described herein. A person of ordinary skill in the art, therefore, will accordingly understand that the technology can have other embodiments with additional elements and that the technology can have other embodiments without several of the features shown and described herein.

After generating double-stranded adapter-fragment DNA complexes comprising, e.g., at least one SMI and at least one SDE, the complex can be subjected to DNA amplification, such as with PCR, or any other biochemical method of DNA amplification, such that one or more copies of the first strand target nucleic acid sequence and one or more copies of the second strand target nucleic acid sequence are produced. The one or more amplification copies of the first strand target nucleic acid molecule and the one or more amplification copies of the second target nucleic acid molecule can then be subjected to DNA sequencing, preferably using a “Next-Generation” massively parallel DNA sequencing platform.

The sequence reads produced from either the first strand target nucleic acid molecule and the second strand target nucleic acid molecule derived from the original double-stranded target nucleic acid molecule can be identified based on sharing a related substantially unique SMI, and in some embodiments (such as embodiments for duplex sequencing) distinguished from the opposite strand target nucleic acid molecule by virtue of an SDE. Once identified, one or more sequence reads produced from the first strand target nucleic acid molecule can be compared with one or more sequence reads produced from the second strand target nucleic acid molecule to produce an error-corrected sequence. For example, nucleotide positions where the bases from both the first and second strand target nucleic acid sequences agree are deemed to be true sequences, whereas nucleotide positions that disagree between the two strands are recognized as potential sites of technical errors that may be discounted. An error-corrected sequence of the original double-stranded target nucleic acid molecule can thus be produced. In some embodiments, one or more sequence reads produced from the first and/or second strand are compared to one another to generate a single-strand consensus sequence (SSCS). In some embodiments, a duplex consensus sequence is obtained by comparing SSCSs for the two strands derived from the same original double-stranded molecule.

In some embodiments, identifying or having identified the eccDNA using the plurality of sequence reads of the duplex sequencing comprises a fragment length analysis. In some embodiments, the fragment length analysis is achieved using a review of the genomic alignment of duplex consensus reads supporting variant calls, as visualized in Integrated Genomics Viewer (IGV) or a similar genome browser. In some embodiments, the review of the genomic alignment of duplex consensus reads supporting variant calls is conducted manually, or using a software.

In some embodiments, analysis is performed to evaluate the presence of D-A junctions. Without wishing to be bound by a particular theory, a junction fusing end and beginning of allele (termed here as “ABCD” allele), subsequently referred to as a “D-A” junction is expected for circular DNA. To identify D-A junctions, the alternate allele sequences of apparent insertions or SVs (as identified through variant calling) may be written in any format, such as but not limited to a FASTA format, which may be used as input for any pattern matching or sequence alignment algorithm, such as but not limited to the matchPattern function from Biostrings (Bioconductor package). The algorithm is used to find the single and/or best match or alignment of the alternate allele sequence the relevant reference sequence and report the reference genome coordinates of the preferred match. In some embodiments, the software is allowed about 1 mismatch per 50 bp. In some embodiments, the algorithm is allowed at most 1 mismatch per 50 bp. In some embodiments, the algorithm is allowed at least 1 mismatch per 50 bp. In some embodiments, the algorithm is allowed about 1 mismatch per 50 bp. The genomic coordinate of the single or best alignment of the alternate allele sequence to the relevant reference sequence are then compared to the start coordinate of the apparent insertion or the apparent structural variant call. If the coordinates of the variant call and of the best alignment of the alternate allele to the reference genome are identical or nearly identical, a D-A junction is present. In some embodiments, the presence of a D-A junction may be further confirmed by inspecting supplementary alignments and/or BLAT-searching any soft-clipped sequences.

In some embodiments, methods and reagents for the enrichment of target nucleic acids material are used, e.g., to limit the detection of eccDNA molecules to one or more genomic regions or loci of interest. For example, in some embodiments, the error-corrected sequences obtained in (b) are specific to a single genomic region. In some embodiments, the error-corrected sequences are specific to 2, 3, 4, 5, or more individual genomic regions. In some embodiments, the error-corrected sequences obtained in (b) are specific to from about 1 to about 30 individual genomic loci. In some embodiments, the error-corrected sequences are specific to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more individual genomic loci. In some embodiments, the error-corrected sequences obtained in (b) are specific to a whole exome.

In some embodiments, and in accordance with aspects of the present technology, sequencing reads generated from the Duplex Sequencing steps discussed herein can be further filtered to eliminate sequencing reads from DNA-damaged molecules (e.g., damaged following tissue or blood extraction). In some embodiments, DNA-damaged molecules (e.g., damaged following tissue or blood extraction) may be removed prior to sequencing with the Duplex Sequencing. For example, DNA repair enzymes, such as Uracil-DNA Glycosylase (UDG), Formamidopyrimidine DNA glycosylase (FPG), and 8-oxoguanine DNA glycosylase (OGG1), can be utilized to correct DNA damage (e.g., in vitro DNA damage). These DNA repair enzymes, for example, are glycosylases that remove damaged bases from DNA. For example, UDG removes uracil that results from cytosine deamination (caused by spontaneous hydrolysis of cytosine) and FPG removes 8-oxo-guanine (e.g., most common DNA lesion that results from reactive oxygen species). FPG also has lyase activity that can generate 1 base gap at abasic sites. Such abasic sites will subsequently fail to amplify by PCR, for example, because the polymerase fails copy the template. In some embodiments, single-stranded DNA gap formed by lyase activity may prevent complete amplification of that strand during PCR. Accordingly, the use of such DNA damage repair enzymes can effectively remove damaged DNA that doesn't have a true mutation, but might otherwise cause an artifactual/erroneous mutation call following sequencing and duplex sequence analysis.

In some embodiments, such glycosylases (e.g., FPG, UDG, OGGI) are used to linearize circular DNA molecules and, e.g., allow their elimination by exonuclease treatment, thereby providing a tool for the detection of DNA damage in eccDNAs.

### V. METHODS

The present disclosure provides for methods of identifying at least one eccDNA in a sample.

In some embodiments, a method of identifying at least one extrachromosomal circular DNA (eccDNA) in a sample comprising double-stranded DNA comprises the step of performing or having performed duplex sequencing on the sample, as provided herein. In some embodiments, the duplex sequencing comprises the step of ligating adaptors to the ends of the double-stranded DNA, as provided herein. In some embodiments, at least one adaptor comprises a nucleotide sequence that tags a strand of the double-stranded DNA such that the strand of the double-stranded DNA has a distinctly identifiable nucleotide sequence relative to its complementary strand. In some embodiments, the duplex sequencing comprises the step of amplifying strands of the double-stranded DNA using the ligated adaptors to generate at least first strand amplicons and second strand amplicons. In some embodiments, the duplex sequencing comprises the step of sequencing at least the first strand amplicons and the second strand amplicons to produce a plurality of sequence reads comprising first strand sequence reads and second strand sequence reads. In some embodiments, the duplex sequencing comprises the step of generating an error-corrected sequence read by comparing the first strand sequence reads and second strand sequence reads by discounting nucleotide positions that do not agree.

In some embodiments, the method of identifying at least one extrachromosomal circular DNA (eccDNA) in a sample comprising double-stranded DNA comprises the step of identifying or having identified the eccDNA using the plurality of sequence reads of the duplex sequencing.

In some embodiments, a method of identifying at least one extrachromosomal circular DNA (eccDNA) in a sample comprising double-stranded DNA, comprises the steps of: performing or having performed duplex sequencing on the sample, wherein the duplex sequencing comprises the steps of: ligating adaptors to the ends of the double-stranded DNA, wherein at least one adaptor comprises a nucleotide sequence that tags a strand of the double-stranded DNA such that the strand of the double-stranded DNA has a distinctly identifiable nucleotide sequence relative to its complementary strand; amplifying strands of the double-stranded DNA using the ligated adaptors to generate at least first strand amplicons and second strand amplicons; sequencing at least the first strand amplicons and the second strand amplicons to produce a plurality of sequence reads comprising first strand sequence reads and second strand sequence reads; and identifying or having identified the eccDNA using the plurality of sequence reads of the duplex sequencing.

In some embodiments, a method of identifying at least one extrachromosomal circular DNA (eccDNA) in a sample comprising double-stranded DNA, comprises the steps of: performing or having performed duplex sequencing on the sample, wherein the duplex sequencing comprises the steps of: ligating adaptors to the ends of the double-stranded DNA, wherein at least one adaptor comprises a nucleotide sequence that tags a strand of the double-stranded DNA such that the strand of the double-stranded DNA has a distinctly identifiable nucleotide sequence relative to its complementary strand; amplifying strands of the double-stranded DNA using the ligated adaptors to generate at least first strand amplicons and second strand amplicons; sequencing at least the first strand amplicons and the second strand amplicons to produce a plurality of sequence reads comprising first strand sequence reads and second strand sequence reads; generating an error-corrected sequence read by comparing the first strand sequence reads and second strand sequence reads by discounting nucleotide positions that do not agree; and identifying or having identified the eccDNA using the plurality of sequence reads of the duplex sequencing.

In some embodiments, a method of identifying at least one extrachromosomal circular DNA (eccDNA) in a sample comprising double-stranded DNA, comprises the steps of: performing or having performed duplex sequencing on the sample, wherein the duplex sequencing comprises the steps of: ligating adaptors to the ends of the double-stranded DNA, wherein at least one adaptor comprises a nucleotide sequence that tags a strand of the double-stranded DNA such that the strand of the double-stranded DNA has a distinctly identifiable nucleotide sequence relative to its complementary strand; amplifying strands of the double-stranded DNA using the ligated adaptors to generate at least first strand amplicons and second strand amplicons; sequencing at least the first strand amplicons and the second strand amplicons to produce a plurality of sequence reads comprising first strand sequence reads and second strand sequence reads; generating an error-corrected sequence read by comparing the first strand sequence reads and second strand sequence reads by discounting nucleotide positions that do not agree; and identifying or having identified the eccDNA using the plurality of sequence reads of the duplex sequencing, wherein the identifying or having identified the eccDNA comprises the steps of: identifying a subset of sequence reads from the plurality of sequence reads, wherein sequence reads of the subset of sequence reads each independently comprise a reference allele junction (e.g., D-A); from amongst the subset of sequence reads, distinguishing putative eccDNA sequence reads from chromosomal tandem duplication sequence reads, wherein distinguishing putative eccDNA sequence reads comprises the steps of: selecting a subset of D-A junction-containing consensus sequencing reads with allele length less than a threshold apparent insert size; inferring the fragment size of each read pair in the subset and comparing it to the threshold apparent insert size, one by one, or comparing the distribution of inferred fragment sizes to the distribution of insert sizes in the entire library; and comparing the inferred fragment size of any one consensus read pair to the allele; and determining a quantity of eccDNA according to the distinguished putative eccDNA sequence reads.

In some embodiments, identifying or having identified the eccDNA using the plurality of sequence reads of the duplex sequencing comprises identifying a subset of sequence reads from the plurality of sequence reads, wherein sequence reads of the subset of sequence reads each independently comprise a reference allele junction. As used herein, the terms “reference allele junction,” “junction,” “D-A junction,” “BA,” and “BA junction” may be used interchangeably, and refer to a nucleic acid sequence of a reference allele comprising a nucleic acid sequence of the end of the reference allele conjugated to a nucleic acid sequence of the beginning of the reference allele.

In some embodiments, identifying or having identified the eccDNA using the plurality of sequence reads of the duplex sequencing comprises from amongst the subset of sequence reads, distinguishing putative eccDNA sequence reads from chromosomal tandem duplication sequence reads. In some embodiments, identifying or having identified the eccDNA using the plurality of sequence reads of the duplex sequencing comprises determining a quantity of eccDNA according to the distinguished putative eccDNA sequence reads.

In some embodiments, identifying or having identified the eccDNA using the plurality of sequence reads of the duplex sequencing comprises identifying a subset of sequence reads from the plurality of sequence reads, wherein sequence reads of the subset of sequence reads each independently comprise a reference allele junction; from amongst the subset of sequence reads, distinguishing putative eccDNA sequence reads from chromosomal tandem duplication sequence reads; and determining a quantity of eccDNA according to the distinguished putative eccDNA sequence reads.

In some embodiments, a method of identifying at least one extrachromosomal circular DNA (eccDNA) in a sample comprising double-stranded DNA, comprises the steps of: performing or having performed duplex sequencing on the sample, wherein the duplex sequencing comprises the steps of: ligating adaptors to the ends of the double-stranded DNA, wherein at least one adaptor comprises a nucleotide sequence that tags a strand of the double-stranded DNA such that the strand of the double-stranded DNA has a distinctly identifiable nucleotide sequence relative to its complementary strand; amplifying strands of the double-stranded DNA using the ligated adaptors to generate at least first strand amplicons and second strand amplicons; sequencing at least the first strand amplicons and the second strand amplicons to produce a plurality of sequence reads comprising first strand sequence reads and second strand sequence reads; generating an error-corrected sequence read by comparing the first strand sequence reads and second strand sequence reads by discounting nucleotide positions that do not agree; and identifying or having identified the eccDNA using the plurality of sequence reads of the duplex sequencing, wherein identifying or having identified the eccDNA using the plurality of sequence reads of the duplex sequencing comprises identifying a subset of sequence reads from the plurality of sequence reads, wherein sequence reads of the subset of sequence reads each independently comprise a reference allele junction; from amongst the subset of sequence reads, distinguishing putative eccDNA sequence reads from chromosomal tandem duplication sequence reads; and determining a quantity of eccDNA according to the distinguished putative eccDNA sequence reads.

In some embodiments, a method for identifying extrachromosomal circular DNA (eccDNA) comprises obtaining a plurality of sequence reads of double-stranded DNA sequenced using duplex sequencing; identifying a subset of sequence reads from the plurality of sequence reads, wherein sequence reads of the subset of sequence reads each independently comprise a reference allele junction; from amongst the subset of sequence reads, distinguishing putative eccDNA sequence reads from chromosomal tandem duplication sequence reads; and identifying eccDNA according to the distinguished putative eccDNA sequence reads.

In some embodiments, a method for identifying extrachromosomal circular DNA (eccDNA) comprises obtaining a plurality of sequence reads of double-stranded DNA sequenced using duplex sequencing; identifying a subset of sequence reads from the plurality of sequence reads, wherein sequence reads of the subset of sequence reads each independently comprise a reference allele junction; from amongst the subset of sequence reads, distinguishing putative eccDNA sequence reads from chromosomal tandem duplication sequence reads, wherein distinguishing putative eccDNA sequence reads comprises the steps of: selecting a subset of D-A junction-containing consensus sequencing reads with allele length less than a threshold apparent insert size; inferring the fragment size of each read pair in the subset and comparing it to the threshold apparent insert size, one by one, or comparing the distribution of inferred fragment sizes to the distribution of insert sizes in the entire library; and comparing the inferred fragment size of any one consensus read pair to the allele; and identifying eccDNA according to the distinguished putative eccDNA sequence reads.

In some embodiments, the at least one adaptor sequence is or comprises at least one non-standard nucleotide. In some embodiments, the non-standard nucleotide is selected from a uracil, a methylated nucleotide, an RNA nucleotide, a ribose nucleotide, an 8-oxo-guanine, a biotinylated nucleotide, a desthiobiotin nucleotide, a thiol modified nucleotide, an acrydite modified nucleotide an iso-dC. an iso dG, a 2′-0-methyl nucleotide, an inosine nucleotide Locked Nucleic Acid, a peptide nucleic acid, a 5 methyl dC, a 5-bromo deoxyuridine, a 2,6-Diaminopurine, 2-Aminopurine nucleotide, an abasic nucleotide, a 5-Nitroindole nucleotide, an adenylated nucleotide, an azide nucleotide, a digoxigenin nucleotide, an I-linker, a 5′ Hexynyl modified nucleotide, an 5-Octadiynyl dU, photocleavable spacer, a non-photocleavable spacer, a click chemistry compatible modified nucleotide, a fluorescent dye, biotin, furan, BrdU, Fluoro-dU, and any combination thereof.

In some embodiments, a reference allele comprises a nucleic acid sequence having a formula ABCD. In some embodiments, the reference allele junction comprises a nucleic acid sequence having a nucleic acid sequence of an end of a reference allele and a nucleic acid sequence of a beginning of a reference allele. In some embodiments, the reference allele junction comprises a nucleic acid sequence having the formula of D-A. In some embodiments, the reference allele junction comprises a nucleic acid sequence having the formula of D-A, wherein B and C are absent. In some embodiments, the reference allele junction comprises a nucleic acid sequence having the formula of D-A, wherein B or C are absent. In some embodiments, the reference allele junction comprises a nucleic acid sequence having the formula of D-A, wherein D comprises the nucleic acid sequence of the end of the reference allele, and A comprises the nucleic acid sequence of the beginning of the reference allele. In some embodiments, the nucleic acid sequence D-A comprises in 5′ to 3′ direction a nucleic acid sequence D operatively conjugated to a nucleic acid sequence A. In some embodiments, the nucleic acid sequence D is located downstream of the nucleic acid sequence A in a reference genomic locus of the reference allele. In some embodiments, the nucleic acid sequence A is located upstream of the nucleic acid sequence D in a reference genomic locus of the reference allele.

In some embodiments, the reference allele junction comprises a nucleic acid sequence that is at least 1 base pairs (bp). In some embodiments, the reference allele junction comprises a nucleic acid sequence that is about 1 bp, 2 bp, 3 bp, 4 bp, 5 bp, 6 bp, 7 bp, 8 bp, 9 bp, 10 bp, 15 bp, 20 bp, about 30 bp, about 40 bp, about 50 bp, about 60 bp, about 70 bp, about 80 bp, about 90 bp, about 100 bp, about 110 bp, about 120 bp, about 130 bp, about 140 bp, about 150 bp, about 160 bp, about 170 bp, about 180 bp, about 190 bp, about 200 bp, about 210 bp, about 220 bp, about 230 bp, about 240 bp, about 250 bp, about 260 bp, about 270 bp, about 280 bp, about 290 bp, about 300 bp, about 310 bp, about 320 bp, about 330 bp, about 340 bp, about 350 bp, about 360 bp, about 370 bp, about 380 bp, about 390 bp, about 400 bp, about 410 bp, about 420 bp, about 430 bp, about 440 bp, about 450 bp, about 460 bp, about 470 bp, about 480 bp, about 490 bp, about 500 bp, about 510 bp, about 520 bp, about 530 bp, about 540 bp, about 550 bp, about 560 bp, about 570 bp, about 580 bp, about 590 bp, about 600 bp, about 610 bp, about 620 bp, about 630 bp, about 640 bp, about 650 bp, about 660 bp, about 670 bp, about 680 bp, about 690 bp, about 700 bp, about 710 bp, about 720 bp, about 730 bp, about 740 bp, about 750 bp, about 760 bp, about 770 bp, about 780 bp, about 790 bp, about 800 bp, about 810 bp, about 820 bp, about 830 bp, about 840 bp, about 850 bp, about 860 bp, about 870 bp, about 880 bp, about 890 bp, about 900 bp, about 910 bp, about 920 bp, about 930 bp, about 940 bp, about 950 bp, about 960 bp, about 970 bp, about 980 bp, about 990 bp, about 1000 bp, or more. In some embodiments, the reference allele junction comprises a nucleic acid sequence that is at least 1 bp, 2 bp, 3 bp, 4 bp, 5 bp, 6 bp, 7 bp, 8 bp, 9 bp, 10 bp, 15 bp, 20 bp, at least 30 bp, at least 40 bp, at least 50 bp, at least 60 bp, at least 70 bp, at least 80 bp, at least 90 bp, at least 100 bp, at least 110 bp, at least 120 bp, at least 130 bp, at least 140 bp, at least 150 bp, at least 160 bp, at least 170 bp, at least 180 bp, at least 190 bp, at least 200 bp, at least 210 bp, at least 220 bp, at least 230 bp, at least 240 bp, at least 250 bp, at least 260 bp, at least 270 bp, at least 280 bp, at least 290 bp, at least 300 bp, at least 310 bp, at least 320 bp, at least 330 bp, at least 340 bp, at least 350 bp, at least 360 bp, at least 370 bp, at least 380 bp, at least 390 bp, at least 400 bp, at least 410 bp, at least 420 bp, at least 430 bp, at least 440 bp, at least 450 bp, at least 460 bp, at least 470 bp, at least 480 bp, at least 490 bp, at least 500 bp, at least 510 bp, at least 520 bp, at least 530 bp, at least 540 bp, at least 550 bp, at least 560 bp, at least 570 bp, at least 580 bp, at least 590 bp, at least 600 bp, at least 610 bp, at least 620 bp, at least 630 bp, at least 640 bp, at least 650 bp, at least 660 bp, at least 670 bp, at least 680 bp, at least 690 bp, at least 700 bp, at least 710 bp, at least 720 bp, at least 730 bp, at least 740 bp, at least 750 bp, at least 760 bp, at least 770 bp, at least 780 bp, at least 790 bp, at least 800 bp, at least 810 bp, at least 820 bp, at least 830 bp, at least 840 bp, at least 850 bp, at least 860 bp, at least 870 bp, at least 880 bp, at least 890 bp, at least 900 bp, at least 910 bp, at least 920 bp, at least 930 bp, at least 940 bp, at least 950 bp, at least 960 bp, at least 970 bp, at least 980 bp, at least 990 bp, at least 1000 bp, or more. In some embodiments, the reference allele junction comprises a nucleic acid sequence that is 1 bp, 2 bp, 3 bp, 4 bp, 5 bp, 6 bp, 7 bp, 8 bp, 9 bp, 10 bp, 15 bp, 20 bp, 30 bp, 40 bp, 50 bp, 60 bp, 70 bp, 80 bp, 90 bp, 100 bp, 110 bp, 120 bp, 130 bp, 140 bp, 150 bp, 160 bp, 170 bp, 180 bp, 190 bp, 200 bp, 210 bp, 220 bp, 230 bp, 240 bp, 250 bp, 260 bp, 270 bp, 280 bp, 290 bp, 300 bp, 310 bp, 320 bp, 330 bp, 340 bp, 350 bp, 360 bp, 370 bp, 380 bp, 390 bp, 400 bp, 410 bp, 420 bp, 430 bp, 440 bp, 450 bp, 460 bp, 470 bp, 480 bp, 490 bp, 500 bp, 510 bp, 520 bp, 530 bp, 540 bp, 550 bp, 560 bp, 570 bp, 580 bp, 590 bp, 600 bp, 610 bp, 620 bp, 630 bp, 640 bp, 650 bp, 660 bp, 670 bp, 680 bp, 690 bp, 700 bp, 710 bp, 720 bp, 730 bp, 740 bp, 750 bp, 760 bp, 770 bp, 780 bp, 790 bp, 800 bp, 810 bp, 820 bp, 830 bp, 840 bp, 850 bp, 860 bp, 870 bp, 880 bp, 890 bp, 900 bp, 910 bp, 920 bp, 930 bp, 940 bp, 950 bp, 960 bp, 970 bp, 980 bp, 990 bp, 1000 bp, or more.

In some embodiments, the reference allele junction comprises a nucleic acid sequence that is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 610%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% the length of the reference allele. In some embodiments, the reference allele junction comprises a nucleic acid sequence that is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% the length of the reference allele. In some embodiments, the reference allele junction comprises a nucleic acid sequence that is 1%, 2%, 3%, 4%, 5%, 6%, 7%, 8%, 9%, 10%, 11%, 12%, 13%, 14%, 15%, 16%, 17%, 18%, %19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83% 84%, 85%, 86%, 87%, 88%, 89% 90%, 91%, 92%, 93%, 94%, 95%, 96%, 97%, 98%, 99%, or 100% the length of the reference allele.

In some embodiments, the nucleic acid sequence D-A is at least 1 base pairs (bp). In some embodiments, the nucleic acid sequence D-A is about 1 bp, 2 bp, 3 bp, 4 bp, 5 bp, 6 bp, 7 bp, 8 bp, 9 bp, 10 bp, 15 bp, 20 bp, about 30 bp, about 40 bp, about 50 bp, about 60 bp, about 70 bp, about 80 bp, about 90 bp, about 100 bp, about 110 bp, about 120 bp, about 130 bp, about 140 bp, about 150 bp, about 160 bp, about 170 bp, about 180 bp, about 190 bp, about 200 bp, about 210 bp, about 220 bp, about 230 bp, about 240 bp, about 250 bp, about 260 bp, about 270 bp, about 280 bp, about 290 bp, about 300 bp, about 310 bp, about 320 bp, about 330 bp, about 340 bp, about 350 bp, about 360 bp, about 370 bp, about 380 bp, about 390 bp, about 400 bp, about 410 bp, about 420 bp, about 430 bp, about 440 bp, about 450 bp, about 460 bp, about 470 bp, about 480 bp, about 490 bp, about 500 bp, about 510 bp, about 520 bp, about 530 bp, about 540 bp, about 550 bp, about 560 bp, about 570 bp, about 580 bp, about 590 bp, about 600 bp, about 610 bp, about 620 bp, about 630 bp, about 640 bp, about 650 bp, about 660 bp, about 670 bp, about 680 bp, about 690 bp, about 700 bp, about 710 bp, about 720 bp, about 730 bp, about 740 bp, about 750 bp, about 760 bp, about 770 bp, about 780 bp, about 790 bp, about 800 bp, about 810 bp, about 820 bp, about 830 bp, about 840 bp, about 850 bp, about 860 bp, about 870 bp, about 880 bp, about 890 bp, about 900 bp, about 910 bp, about 920 bp, about 930 bp, about 940 bp, about 950 bp, about 960 bp, about 970 bp, about 980 bp, about 990 bp, about 1000 bp, or more. In some embodiments, the nucleic acid sequence D-A is at least 1 bp, 2 bp, 3 bp, 4 bp, 5 bp, 6 bp, 7 bp, 8 bp, 9 bp, 10 bp, 15 bp, 20 bp, at least 30 bp, at least 40 bp, at least 50 bp, at least 60 bp, at least 70 bp, at least 80 bp, at least 90 bp, at least 100 bp, at least 110 bp, at least 120 bp, at least 130 bp, at least 140 bp, at least 150 bp, at least 160 bp, at least 170 bp, at least 180 bp, at least 190 bp, at least 200 bp, at least 210 bp, at least 220 bp, at least 230 bp, at least 240 bp, at least 250 bp, at least 260 bp, at least 270 bp, at least 280 bp, at least 290 bp, at least 300 bp, at least 310 bp, at least 320 bp, at least 330 bp, at least 340 bp, at least 350 bp, at least 360 bp, at least 370 bp, at least 380 bp, at least 390 bp, at least 400 bp, at least 410 bp, at least 420 bp, at least 430 bp, at least 440 bp, at least 450 bp, at least 460 bp, at least 470 bp, at least 480 bp, at least 490 bp, at least 500 bp, at least 510 bp, at least 520 bp, at least 530 bp, at least 540 bp, at least 550 bp, at least 560 bp, at least 570 bp, at least 580 bp, at least 590 bp, at least 600 bp, at least 610 bp, at least 620 bp, at least 630 bp, at least 640 bp, at least 650 bp, at least 660 bp, at least 670 bp, at least 680 bp, at least 690 bp, at least 700 bp, at least 710 bp, at least 720 bp, at least 730 bp, at least 740 bp, at least 750 bp, at least 760 bp, at least 770 bp, at least 780 bp, at least 790 bp, at least 800 bp, at least 810 bp, at least 820 bp, at least 830 bp, at least 840 bp, at least 850 bp, at least 860 bp, at least 870 bp, at least 880 bp, at least 890 bp, at least 900 bp, at least 910 bp, at least 920 bp, at least 930 bp, at least 940 bp, at least 950 bp, at least 960 bp, at least 970 bp, at least 980 bp, at least 990 bp, at least 1000 bp, or more. In some embodiments, the nucleic acid sequence D-A is 1 bp, 2 bp, 3 bp, 4 bp, 5 bp, 6 bp, 7 bp, 8 bp, 9 bp, 10 bp, 15 bp, 20 bp, 30 bp, 40 bp, 50 bp, 60 bp, 70 bp, 80 bp, 90 bp, 100 bp, 110 bp, 120 bp, 130 bp, 140 bp, 150 bp, 160 bp, 170 bp, 180 bp, 190 bp, 200 bp, 210 bp, 220 bp, 230 bp, 240 bp, 250 bp, 260 bp, 270 bp, 280 bp, 290 bp, 300 bp, 310 bp, 320 bp, 330 bp, 340 bp, 350 bp, 360 bp, 370 bp, 380 bp, 390 bp, 400 bp, 410 bp, 420 bp, 430 bp, 440 bp, 450 bp, 460 bp, 470 bp, 480 bp, 490 bp, 500 bp, 510 bp, 520 bp, 530 bp, 540 bp, 550 bp, 560 bp, 570 bp, 580 bp, 590 bp, 600 bp, 610 bp, 620 bp, 630 bp, 640 bp, 650 bp, 660 bp, 670 bp, 680 bp, 690 bp, 700 bp, 710 bp, 720 bp, 730 bp, 740 bp, 750 bp, 760 bp, 770 bp, 780 bp, 790 bp, 800 bp, 810 bp, 820 bp, 830 bp, 840 bp, 850 bp, 860 bp, 870 bp, 880 bp, 890 bp, 900 bp, 910 bp, 920 bp, 930 bp, 940 bp, 950 bp, 960 bp, 970 bp, 980 bp, 990 bp, 1000 bp, or more.

In some embodiments, the nucleic acid sequence D-A is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% the length of the reference allele. In some embodiments, the nucleic acid sequence D-A is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% the length of the reference allele. In some embodiments, the nucleic acid sequence D-A is 1%, 2%, 3%, 4%, 5%, 6%, 7%, 8%, 9%, 10%, 11%, 12%, 13%, 14%, 15%, 16%, 17%, 18%, %19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% the length of the reference allele.

In some embodiments, the nucleic acid sequence D is at least 1 base pairs (bp). In some embodiments, the nucleic acid sequence D is about 1 bp, 2 bp, 3 bp, 4 bp, 5 bp, 6 bp, 7 bp, 8 bp, 9 bp, 10 bp, 15 bp, 20 bp, about 30 bp, about 40 bp, about 50 bp, about 60 bp, about 70 bp, about 80 bp, about 90 bp, about 100 bp, about 110 bp, about 120 bp, about 130 bp, about 140 bp, about 150 bp, about 160 bp, about 170 bp, about 180 bp, about 190 bp, about 200 bp, about 210 bp, about 220 bp, about 230 bp, about 240 bp, about 250 bp, about 260 bp, about 270 bp, about 280 bp, about 290 bp, about 300 bp, about 310 bp, about 320 bp, about 330 bp, about 340 bp, about 350 bp, about 360 bp, about 370 bp, about 380 bp, about 390 bp, about 400 bp, about 410 bp, about 420 bp, about 430 bp, about 440 bp, about 450 bp, about 460 bp, about 470 bp, about 480 bp, about 490 bp, about 500 bp, about 510 bp, about 520 bp, about 530 bp, about 540 bp, about 550 bp, about 560 bp, about 570 bp, about 580 bp, about 590 bp, about 600 bp, about 610 bp, about 620 bp, about 630 bp, about 640 bp, about 650 bp, about 660 bp, about 670 bp, about 680 bp, about 690 bp, about 700 bp, about 710 bp, about 720 bp, about 730 bp, about 740 bp, about 750 bp, about 760 bp, about 770 bp, about 780 bp, about 790 bp, about 800 bp, about 810 bp, about 820 bp, about 830 bp, about 840 bp, about 850 bp, about 860 bp, about 870 bp, about 880 bp, about 890 bp, about 900 bp, about 910 bp, about 920 bp, about 930 bp, about 940 bp, about 950 bp, about 960 bp, about 970 bp, about 980 bp, about 990 bp, about 1000 bp, or more. In some embodiments, the nucleic acid sequence D is at least 1 bp, 2 bp, 3 bp, 4 bp, 5 bp, 6 bp, 7 bp, 8 bp, 9 bp, 10 bp, 15 bp, 20 bp, at least 30 bp, at least 40 bp, at least 50 bp, at least 60 bp, at least 70 bp, at least 80 bp, at least 90 bp, at least 100 bp, at least 110 bp, at least 120 bp, at least 130 bp, at least 140 bp, at least 150 bp, at least 160 bp, at least 170 bp, at least 180 bp, at least 190 bp, at least 200 bp, at least 210 bp, at least 220 bp, at least 230 bp, at least 240 bp, at least 250 bp, at least 260 bp, at least 270 bp, at least 280 bp, at least 290 bp, at least 300 bp, at least 310 bp, at least 320 bp, at least 330 bp, at least 340 bp, at least 350 bp, at least 360 bp, at least 370 bp, at least 380 bp, at least 390 bp, at least 400 bp, at least 410 bp, at least 420 bp, at least 430 bp, at least 440 bp, at least 450 bp, at least 460 bp, at least 470 bp, at least 480 bp, at least 490 bp, at least 500 bp, at least 510 bp, at least 520 bp, at least 530 bp, at least 540 bp, at least 550 bp, at least 560 bp, at least 570 bp, at least 580 bp, at least 590 bp, at least 600 bp, at least 610 bp, at least 620 bp, at least 630 bp, at least 640 bp, at least 650 bp, at least 660 bp, at least 670 bp, at least 680 bp, at least 690 bp, at least 700 bp, at least 710 bp, at least 720 bp, at least 730 bp, at least 740 bp, at least 750 bp, at least 760 bp, at least 770 bp, at least 780 bp, at least 790 bp, at least 800 bp, at least 810 bp, at least 820 bp, at least 830 bp, at least 840 bp, at least 850 bp, at least 860 bp, at least 870 bp, at least 880 bp, at least 890 bp, at least 900 bp, at least 910 bp, at least 920 bp, at least 930 bp, at least 940 bp, at least 950 bp, at least 960 bp, at least 970 bp, at least 980 bp, at least 990 bp, at least 1000 bp, or more. In some embodiments, the nucleic acid sequence D is 1 bp, 2 bp, 3 bp, 4 bp, 5 bp, 6 bp, 7 bp, 8 bp, 9 bp, 10 bp, 15 bp, 20 bp, 30 bp, 40 bp, 50 bp, 60 bp, 70 bp, 80 bp, 90 bp, 100 bp, 110 bp, 120 bp, 130 bp, 140 bp, 150 bp, 160 bp, 170 bp, 180 bp, 190 bp, 200 bp, 210 bp, 220 bp, 230 bp, 240 bp, 250 bp, 260 bp, 270 bp, 280 bp, 290 bp, 300 bp, 310 bp, 320 bp, 330 bp, 340 bp, 350 bp, 360 bp, 370 bp, 380 bp, 390 bp, 400 bp, 410 bp, 420 bp, 430 bp, 440 bp, 450 bp, 460 bp, 470 bp, 480 bp, 490 bp, 500 bp, 510 bp, 520 bp, 530 bp, 540 bp, 550 bp, 560 bp, 570 bp, 580 bp, 590 bp, 600 bp, 610 bp, 620 bp, 630 bp, 640 bp, 650 bp, 660 bp, 670 bp, 680 bp, 690 bp, 700 bp, 710 bp, 720 bp, 730 bp, 740 bp, 750 bp, 760 bp, 770 bp, 780 bp, 790 bp, 800 bp, 810 bp, 820 bp, 830 bp, 840 bp, 850 bp, 860 bp, 870 bp, 880 bp, 890 bp, 900 bp, 910 bp, 920 bp, 930 bp, 940 bp, 950 bp, 960 bp, 970 bp, 980 bp, 990 bp, 1000 bp, or more.

In some embodiments, the nucleic acid sequence D is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% the length of the reference allele. In some embodiments, the nucleic acid sequence D is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% the length of the reference allele. In some embodiments, the nucleic acid sequence D is 1%, 2%, 3%, 4%, 5%, 6%, 7%, 8%, 9%, 10%, 11%, 12%, 13%, 14%, 15%, 16%, 17% 18%, %19%, 20%, 21%, 22%, 23%, 24%, 25%, 26%, 27%, 28%, 29%, 30%, 31%, 32%, 33% 34% 35%, 36%, 37%, 38%, 39% 40% 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85% 86%, 87%, 88%, 89%, 90%, 91% 92%, 93%, 94%, 95%, 96%, 97%, 98%, 99%, or 100% the length of the reference allele.

In some embodiments, the nucleic acid sequence A is at least 1 base pairs (bp). In some embodiments, the nucleic acid sequence A is about 1 bp, 2 bp, 3 bp, 4 bp, 5 bp, 6 bp, 7 bp, 8 bp, 9 bp, 10 bp, 15 bp, 20 bp, about 30 bp, about 40 bp, about 50 bp, about 60 bp, about 70 bp, about 80 bp, about 90 bp, about 100 bp, about 110 bp, about 120 bp, about 130 bp, about 140 bp, about 150 bp, about 160 bp, about 170 bp, about 180 bp, about 190 bp, about 200 bp, about 210 bp, about 220 bp, about 230 bp, about 240 bp, about 250 bp, about 260 bp, about 270 bp, about 280 bp, about 290 bp, about 300 bp, about 310 bp, about 320 bp, about 330 bp, about 340 bp, about 350 bp, about 360 bp, about 370 bp, about 380 bp, about 390 bp, about 400 bp, about 410 bp, about 420 bp, about 430 bp, about 440 bp, about 450 bp, about 460 bp, about 470 bp, about 480 bp, about 490 bp, about 500 bp, about 510 bp, about 520 bp, about 530 bp, about 540 bp, about 550 bp, about 560 bp, about 570 bp, about 580 bp, about 590 bp, about 600 bp, about 610 bp, about 620 bp, about 630 bp, about 640 bp, about 650 bp, about 660 bp, about 670 bp, about 680 bp, about 690 bp, about 700 bp, about 710 bp, about 720 bp, about 730 bp, about 740 bp, about 750 bp, about 760 bp, about 770 bp, about 780 bp, about 790 bp, about 800 bp, about 810 bp, about 820 bp, about 830 bp, about 840 bp, about 850 bp, about 860 bp, about 870 bp, about 880 bp, about 890 bp, about 900 bp, about 910 bp, about 920 bp, about 930 bp, about 940 bp, about 950 bp, about 960 bp, about 970 bp, about 980 bp, about 990 bp, about 1000 bp, or more. In some embodiments, the nucleic acid sequence A is at least at least 1 bp, 2 bp, 3 bp, 4 bp, 5 bp, 6 bp, 7 bp, 8 bp, 9 bp, 10 bp, 15 bp, 20 bp, at least 30 bp, at least 40 bp, at least 50 bp, at least 60 bp, at least 70 bp, at least 80 bp, at least 90 bp, at least 100 bp, at least 110 bp, at least 120 bp, at least 130 bp, at least 140 bp, at least 150 bp, at least 160 bp, at least 170 bp, at least 180 bp, at least 190 bp, at least 200 bp, at least 210 bp, at least 220 bp, at least 230 bp, at least 240 bp, at least 250 bp, at least 260 bp, at least 270 bp, at least 280 bp, at least 290 bp, at least 300 bp, at least 310 bp, at least 320 bp, at least 330 bp, at least 340 bp, at least 350 bp, at least 360 bp, at least 370 bp, at least 380 bp, at least 390 bp, at least 400 bp, at least 410 bp, at least 420 bp, at least 430 bp, at least 440 bp, at least 450 bp, at least 460 bp, at least 470 bp, at least 480 bp, at least 490 bp, at least 500 bp, at least 510 bp, at least 520 bp, at least 530 bp, at least 540 bp, at least 550 bp, at least 560 bp, at least 570 bp, at least 580 bp, at least 590 bp, at least 600 bp, at least 610 bp, at least 620 bp, at least 630 bp, at least 640 bp, at least 650 bp, at least 660 bp, at least 670 bp, at least 680 bp, at least 690 bp, at least 700 bp, at least 710 bp, at least 720 bp, at least 730 bp, at least 740 bp, at least 750 bp, at least 760 bp, at least 770 bp, at least 780 bp, at least 790 bp, at least 800 bp, at least 810 bp, at least 820 bp, at least 830 bp, at least 840 bp, at least 850 bp, at least 860 bp, at least 870 bp, at least 880 bp, at least 890 bp, at least 900 bp, at least 910 bp, at least 920 bp, at least 930 bp, at least 940 bp, at least 950 bp, at least 960 bp, at least 970 bp, at least 980 bp, at least 990 bp, at least 1000 bp, or more. In some embodiments, the nucleic acid sequence A is 1 bp, 2 bp, 3 bp, 4 bp, 5 bp, 6 bp, 7 bp, 8 bp, 9 bp, 10 bp, 15 bp, 20 bp, 30 bp, 40 bp, 50 bp, 60 bp, 70 bp, 80 bp, 90 bp, 100 bp, 110 bp, 120 bp, 130 bp, 140 bp, 150 bp, 160 bp, 170 bp, 180 bp, 190 bp, 200 bp, 210 bp, 220 bp, 230 bp, 240 bp, 250 bp, 260 bp, 270 bp, 280 bp, 290 bp, 300 bp, 310 bp, 320 bp, 330 bp, 340 bp, 350 bp, 360 bp, 370 bp, 380 bp, 390 bp, 400 bp, 410 bp, 420 bp, 430 bp, 440 bp, 450 bp, 460 bp, 470 bp, 480 bp, 490 bp, 500 bp, 510 bp, 520 bp, 530 bp, 540 bp, 550 bp, 560 bp, 570 bp, 580 bp, 590 bp, 600 bp, 610 bp, 620 bp, 630 bp, 640 bp, 650 bp, 660 bp, 670 bp, 680 bp, 690 bp, 700 bp, 710 bp, 720 bp, 730 bp, 740 bp, 750 bp, 760 bp, 770 bp, 780 bp, 790 bp, 800 bp, 810 bp, 820 bp, 830 bp, 840 bp, 850 bp, 860 bp, 870 bp, 880 bp, 890 bp, 900 bp, 910 bp, 920 bp, 930 bp, 940 bp, 950 bp, 960 bp, 970 bp, 980 bp, 990 bp, 1000 bp, or more.

In some embodiments, the nucleic acid sequence A is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% the length of the reference allele. In some embodiments, the nucleic acid sequence A is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% the length of the reference allele. In some embodiments, the nucleic acid sequence A is 1%, 2%, 3%, 4%, 5%, 6%, 7% 8%, 9%, 10%, 11%, 12%, 13%, 14%, 15%, 16%, 17% 18% 19% 20%, 21%, 22%, 23% 24%, 25%, 26%, 27%, 28%, 29%, 30%, 31%, 32%, 33%, 34%, 35%, 36%, 37%, 38%, 39% 40% 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% the length of the reference allele.

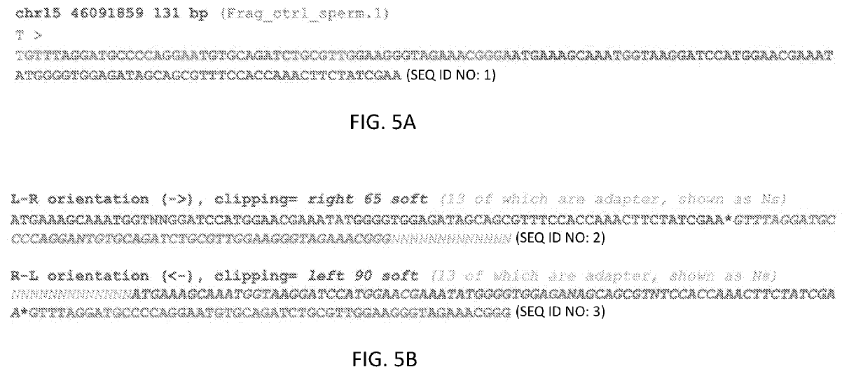

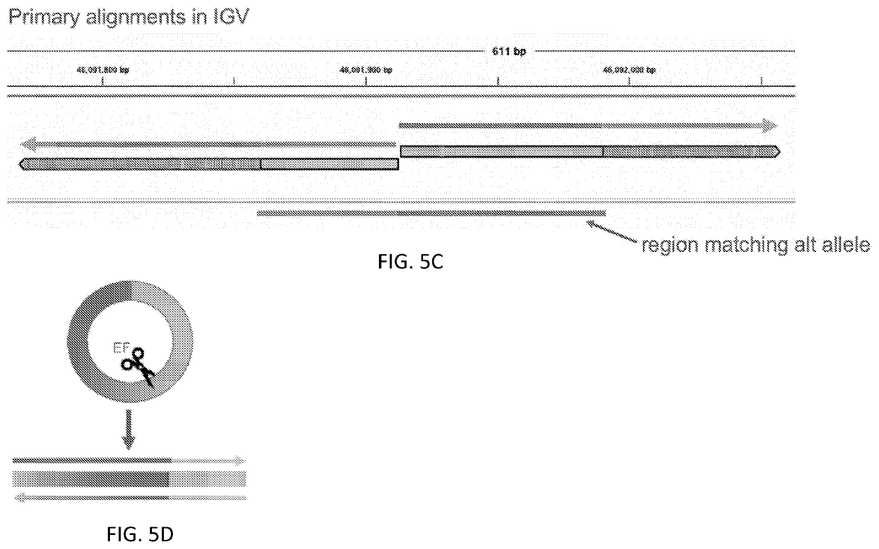

In some embodiments, distinguishing putative eccDNA sequence reads from chromosomal tandem duplication sequence reads comprises the step of comparing inferred fragment sizes of the subset of sequence reads against a threshold value derived from the insert size distribution of all sequencing reads in the library. Insert size is a metric generated during alignment of reads/consensus reads to a reference genome. In most sequencing libraries, a vast majority of read pairs (from paired-end sequencing) map concordantly (R1 maps upstream of R2) and the calculated insert size is equivalent to the size of the DNA fragment before adapter ligation. Therefore, the distribution of insert sizes of a sequencing library is a good approximation of the distribution of fragment sizes of source DNA after fragmentation and prior to adapter ligation. However, most (consensus) read pairs arising from D-A junction-containing molecules align discordantly (with R2 mapping upstream of R1, as shown in FIG. 5C). For discordant read pairs, the insert size calculated by the alignment software does not accurately represent the length of the original DNA fragment. The fragment length must be inferred by manual or computational reconstruction of the structure and sequence of the original DNA fragment.

In some embodiments, distinguishing putative eccDNA sequence reads from chromosomal tandem duplication sequence reads further comprises the step of selecting a subset of D-A junction-containing consensus sequencing reads with allele length less than a threshold apparent insert size. In some embodiments, distinguishing putative eccDNA sequence reads from chromosomal tandem duplication sequence reads further comprises the step of inferring the fragment size of each read pair in the subset and comparing it to the threshold apparent insert size, one by one, or comparing the distribution of inferred fragment sizes to the distribution of insert sizes in the entire library. In some embodiments, distinguishing putative eccDNA sequence reads from chromosomal tandem duplication sequence reads further comprises the steps of selecting a subset of D-A junction-containing consensus sequencing reads with allele length less than a threshold apparent insert size; and inferring the fragment size of each read pair in the subset and comparing it to the threshold apparent insert size, one by one, or comparing the distribution of inferred fragment sizes to the distribution of insert sizes in the entire library.

In some embodiments, distinguishing putative eccDNA sequence reads from chromosomal tandem duplication sequence reads further comprises the step of comparing the inferred fragment size of any one consensus read pair to the allele size of that read pair.

In some embodiments, distinguishing putative eccDNA sequence reads from chromosomal tandem duplication sequence reads further comprises the steps of selecting a subset of D-A junction-containing consensus sequencing reads with allele length less than a threshold apparent insert size; inferring the fragment size of each read pair in the subset and comparing it to the threshold apparent insert size, one by one, or comparing the distribution of inferred fragment sizes to the distribution of insert sizes in the entire library; and comparing the inferred fragment size of any one consensus read pair to the allele size of that read pair.

In some embodiments, the threshold apparent insert size is about 20 base pair (bp). In some embodiments, the threshold apparent insert size is at least 20 base pair (bp). In some embodiments, the threshold apparent insert size is about 20 bp, about 30 bp, about 40 bp, about 50 bp, about 60 bp, about 70 bp, about 80 bp, about 90 bp, about 100 bp, about 110 bp, about 120 bp, about 130 bp, about 140 bp, about 150 bp, about 160 bp, about 170 bp, about 180 bp, about 190 bp, about 200 bp, about 210 bp, about 220 bp, about 230 bp, about 240 bp, about 250 bp, about 260 bp, about 270 bp, about 280 bp, about 290 bp, about 300 bp, about 310 bp, about 320 bp, about 330 bp, about 340 bp, about 350 bp, about 360 bp, about 370 bp, about 380 bp, about 390 bp, about 400 bp, about 410 bp, about 420 bp, about 430 bp, about 440 bp, about 450 bp, about 460 bp, about 470 bp, about 480 bp, about 490 bp, about 500 bp, about 510 bp, about 520 bp, about 530 bp, about 540 bp, about 550 bp, about 560 bp, about 570 bp, about 580 bp, about 590 bp, about 600 bp, about 610 bp, about 620 bp, about 630 bp, about 640 bp, about 650 bp, about 660 bp, about 670 bp, about 680 bp, about 690 bp, about 700 bp, about 710 bp, about 720 bp, about 730 bp, about 740 bp, about 750 bp, about 760 bp, about 770 bp, about 780 bp, about 790 bp, about 800 bp, about 810 bp, about 820 bp, about 830 bp, about 840 bp, about 850 bp, about 860 bp, about 870 bp, about 880 bp, about 890 bp, about 900 bp, about 910 bp, about 920 bp, about 930 bp, about 940 bp, about 950 bp, about 960 bp, about 970 bp, about 980 bp, about 990 bp, about 1000 bp, or more. In some embodiments, the threshold apparent insert size is at least 20 bp, at least 30 bp, at least 40 bp, at least 50 bp, at least 60 bp, at least 70 bp, at least 80 bp, at least 90 bp, at least 100 bp, at least 110 bp, at least 120 bp, at least 130 bp, at least 140 bp, at least 150 bp, at least 160 bp, at least 170 bp, at least 180 bp, at least 190 bp, at least 200 bp, at least 210 bp, at least 220 bp, at least 230 bp, at least 240 bp, at least 250 bp, at least 260 bp, at least 270 bp, at least 280 bp, at least 290 bp, at least 300 bp, at least 310 bp, at least 320 bp, at least 330 bp, at least 340 bp, at least 350 bp, at least 360 bp, at least 370 bp, at least 380 bp, at least 390 bp, at least 400 bp, at least 410 bp, at least 420 bp, at least 430 bp, at least 440 bp, at least 450 bp, at least 460 bp, at least 470 bp, at least 480 bp, at least 490 bp, at least 500 bp, at least 510 bp, at least 520 bp, at least 530 bp, at least 540 bp, at least 550 bp, at least 560 bp, at least 570 bp, at least 580 bp, at least 590 bp, at least 600 bp, at least 610 bp, at least 620 bp, at least 630 bp, at least 640 bp, at least 650 bp, at least 660 bp, at least 670 bp, at least 680 bp, at least 690 bp, at least 700 bp, at least 710 bp, at least 720 bp, at least 730 bp, at least 740 bp, at least 750 bp, at least 760 bp, at least 770 bp, at least 780 bp, at least 790 bp, at least 800 bp, at least 810 bp, at least 820 bp, at least 830 bp, at least 840 bp, at least 850 bp, at least 860 bp, at least 870 bp, at least 880 bp, at least 890 bp, at least 900 bp, at least 910 bp, at least 920 bp, at least 930 bp, at least 940 bp, at least 950 bp, at least 960 bp, at least 970 bp, at least 980 bp, at least 990 bp, at least 1000 bp, or more. In some embodiments, the threshold apparent insert size is 20 bp, 30 bp, 40 bp, 50 bp, 60 bp, 70 bp, 80 bp, 90 bp, 100 bp, 110 bp, 120 bp, 130 bp, 140 bp, 150 bp, 160 bp, 170 bp, 180 bp, 190 bp, 200 bp, 210 bp, 220 bp, 230 bp, 240 bp, 250 bp, 260 bp, 270 bp, 280 bp, 290 bp, 300 bp, 310 bp, 320 bp, 330 bp, 340 bp, 350 bp, 360 bp, 370 bp, 380 bp, 390 bp, 400 bp, 410 bp, 420 bp, 430 bp, 440 bp, 450 bp, 460 bp, 470 bp, 480 bp, 490 bp, 500 bp, 510 bp, 520 bp, 530 bp, 540 bp, 550 bp, 560 bp, 570 bp, 580 bp, 590 bp, 600 bp, 610 bp, 620 bp, 630 bp, 640 bp, 650 bp, 660 bp, 670 bp, 680 bp, 690 bp, 700 bp, 710 bp, 720 bp, 730 bp, 740 bp, 750 bp, 760 bp, 770 bp, 780 bp, 790 bp, 800 bp, 810 bp, 820 bp, 830 bp, 840 bp, 850 bp, 860 bp, 870 bp, 880 bp, 890 bp, 900 bp, 910 bp, 920 bp, 930 bp, 940 bp, 950 bp, 960 bp, 970 bp, 980 bp, 990 bp, 1000 bp, or more.

In some embodiments, the inferred fragment size is about 20 base pair (bp). In some embodiments, the threshold apparent insert size is at least 20 base pair (bp). In some embodiments, the threshold apparent insert size is about 20 bp, about 30 bp, about 40 bp, about 50 bp, about 60 bp, about 70 bp, about 80 bp, about 90 bp, about 100 bp, about 110 bp, about 120 bp, about 130 bp, about 140 bp, about 150 bp, about 160 bp, about 170 bp, about 180 bp, about 190 bp, about 200 bp, about 210 bp, about 220 bp, about 230 bp, about 240 bp, about 250 bp, about 260 bp, about 270 bp, about 280 bp, about 290 bp, about 300 bp, about 310 bp, about 320 bp, about 330 bp, about 340 bp, about 350 bp, about 360 bp, about 370 bp, about 380 bp, about 390 bp, about 400 bp, about 410 bp, about 420 bp, about 430 bp, about 440 bp, about 450 bp, about 460 bp, about 470 bp, about 480 bp, about 490 bp, about 500 bp, about 510 bp, about 520 bp, about 530 bp, about 540 bp, about 550 bp, about 560 bp, about 570 bp, about 580 bp, about 590 bp, about 600 bp, about 610 bp, about 620 bp, about 630 bp, about 640 bp, about 650 bp, about 660 bp, about 670 bp, about 680 bp, about 690 bp, about 700 bp, about 710 bp, about 720 bp, about 730 bp, about 740 bp, about 750 bp, about 760 bp, about 770 bp, about 780 bp, about 790 bp, about 800 bp, about 810 bp, about 820 bp, about 830 bp, about 840 bp, about 850 bp, about 860 bp, about 870 bp, about 880 bp, about 890 bp, about 900 bp, about 910 bp, about 920 bp, about 930 bp, about 940 bp, about 950 bp, about 960 bp, about 970 bp, about 980 bp, about 990 bp, about 1000 bp, or more. In some embodiments, the inferred fragment size is at least 20 bp, at least 30 bp, at least 40 bp, at least 50 bp, at least 60 bp, at least 70 bp, at least 80 bp, at least 90 bp, at least 100 bp, at least 110 bp, at least 120 bp, at least 130 bp, at least 140 bp, at least 150 bp, at least 160 bp, at least 170 bp, at least 180 bp, at least 190 bp, at least 200 bp, at least 210 bp, at least 220 bp, at least 230 bp, at least 240 bp, at least 250 bp, at least 260 bp, at least 270 bp, at least 280 bp, at least 290 bp, at least 300 bp, at least 310 bp, at least 320 bp, at least 330 bp, at least 340 bp, at least 350 bp, at least 360 bp, at least 370 bp, at least 380 bp, at least 390 bp, at least 400 bp, at least 410 bp, at least 420 bp, at least 430 bp, at least 440 bp, at least 450 bp, at least 460 bp, at least 470 bp, at least 480 bp, at least 490 bp, at least 500 bp, at least 510 bp, at least 520 bp, at least 530 bp, at least 540 bp, at least 550 bp, at least 560 bp, at least 570 bp, at least 580 bp, at least 590 bp, at least 600 bp, at least 610 bp, at least 620 bp, at least 630 bp, at least 640 bp, at least 650 bp, at least 660 bp, at least 670 bp, at least 680 bp, at least 690 bp, at least 700 bp, at least 710 bp, at least 720 bp, at least 730 bp, at least 740 bp, at least 750 bp, at least 760 bp, at least 770 bp, at least 780 bp, at least 790 bp, at least 800 bp, at least 810 bp, at least 820 bp, at least 830 bp, at least 840 bp, at least 850 bp, at least 860 bp, at least 870 bp, at least 880 bp, at least 890 bp, at least 900 bp, at least 910 bp, at least 920 bp, at least 930 bp, at least 940 bp, at least 950 bp, at least 960 bp, at least 970 bp, at least 980 bp, at least 990 bp, at least 1000 bp, or more. In some embodiments, the inferred fragment size is 20 bp, 30 bp, 40 bp, 50 bp, 60 bp, 70 bp, 80 bp, 90 bp, 100 bp, 110 bp, 120 bp, 130 bp, 140 bp, 150 bp, 160 bp, 170 bp, 180 bp, 190 bp, 200 bp, 210 bp, 220 bp, 230 bp, 240 bp, 250 bp, 260 bp, 270 bp, 280 bp, 290 bp, 300 bp, 310 bp, 320 bp, 330 bp, 340 bp, 350 bp, 360 bp, 370 bp, 380 bp, 390 bp, 400 bp, 410 bp, 420 bp, 430 bp, 440 bp, 450 bp, 460 bp, 470 bp, 480 bp, 490 bp, 500 bp, 510 bp, 520 bp, 530 bp, 540 bp, 550 bp, 560 bp, 570 bp, 580 bp, 590 bp, 600 bp, 610 bp, 620 bp, 630 bp, 640 bp, 650 bp, 660 bp, 670 bp, 680 bp, 690 bp, 700 bp, 710 bp, 720 bp, 730 bp, 740 bp, 750 bp, 760 bp, 770 bp, 780 bp, 790 bp, 800 bp, 810 bp, 820 bp, 830 bp, 840 bp, 850 bp, 860 bp, 870 bp, 880 bp, 890 bp, 900 bp, 910 bp, 920 bp, 930 bp, 940 bp, 950 bp, 960 bp, 970 bp, 980 bp, 990 bp, 1000 bp, or more.

In some embodiments, the putative eccDNA is identified as having the inferred fragment size less than or equal to the allele length. In some embodiments, the inferred fragment size is about 20 base pair (bp). In some embodiments, the allele length is at least 20 base pair (bp). In some embodiments, the allele length is about 20 bp, about 30 bp, about 40 bp, about 50 bp, about 60 bp, about 70 bp, about 80 bp, about 90 bp, about 100 bp, about 110 bp, about 120 bp, about 130 bp, about 140 bp, about 150 bp, about 160 bp, about 170 bp, about 180 bp, about 190 bp, about 200 bp, about 210 bp, about 220 bp, about 230 bp, about 240 bp, about 250 bp, about 260 bp, about 270 bp, about 280 bp, about 290 bp, about 300 bp, about 310 bp, about 320 bp, about 330 bp, about 340 bp, about 350 bp, about 360 bp, about 370 bp, about 380 bp, about 390 bp, about 400 bp, about 410 bp, about 420 bp, about 430 bp, about 440 bp, about 450 bp, about 460 bp, about 470 bp, about 480 bp, about 490 bp, about 500 bp, about 510 bp, about 520 bp, about 530 bp, about 540 bp, about 550 bp, about 560 bp, about 570 bp, about 580 bp, about 590 bp, about 600 bp, about 610 bp, about 620 bp, about 630 bp, about 640 bp, about 650 bp, about 660 bp, about 670 bp, about 680 bp, about 690 bp, about 700 bp, about 710 bp, about 720 bp, about 730 bp, about 740 bp, about 750 bp, about 760 bp, about 770 bp, about 780 bp, about 790 bp, about 800 bp, about 810 bp, about 820 bp, about 830 bp, about 840 bp, about 850 bp, about 860 bp, about 870 bp, about 880 bp, about 890 bp, about 900 bp, about 910 bp, about 920 bp, about 930 bp, about 940 bp, about 950 bp, about 960 bp, about 970 bp, about 980 bp, about 990 bp, about 1000 bp, or more. In some embodiments, the allele length is at least 20 bp, at least 30 bp, at least 40 bp, at least 50 bp, at least 60 bp, at least 70 bp, at least 80 bp, at least 90 bp, at least 100 bp, at least 110 bp, at least 120 bp, at least 130 bp, at least 140 bp, at least 150 bp, at least 160 bp, at least 170 bp, at least 180 bp, at least 190 bp, at least 200 bp, at least 210 bp, at least 220 bp, at least 230 bp, at least 240 bp, at least 250 bp, at least 260 bp, at least 270 bp, at least 280 bp, at least 290 bp, at least 300 bp, at least 310 bp, at least 320 bp, at least 330 bp, at least 340 bp, at least 350 bp, at least 360 bp, at least 370 bp, at least 380 bp, at least 390 bp, at least 400 bp, at least 410 bp, at least 420 bp, at least 430 bp, at least 440 bp, at least 450 bp, at least 460 bp, at least 470 bp, at least 480 bp, at least 490 bp, at least 500 bp, at least 510 bp, at least 520 bp, at least 530 bp, at least 540 bp, at least 550 bp, at least 560 bp, at least 570 bp, at least 580 bp, at least 590 bp, at least 600 bp, at least 610 bp, at least 620 bp, at least 630 bp, at least 640 bp, at least 650 bp, at least 660 bp, at least 670 bp, at least 680 bp, at least 690 bp, at least 700 bp, at least 710 bp, at least 720 bp, at least 730 bp, at least 740 bp, at least 750 bp, at least 760 bp, at least 770 bp, at least 780 bp, at least 790 bp, at least 800 bp, at least 810 bp, at least 820 bp, at least 830 bp, at least 840 bp, at least 850 bp, at least 860 bp, at least 870 bp, at least 880 bp, at least 890 bp, at least 900 bp, at least 910 bp, at least 920 bp, at least 930 bp, at least 940 bp, at least 950 bp, at least 960 bp, at least 970 bp, at least 980 bp, at least 990 bp, at least 1000 bp, or more. In some embodiments, the allele length is 20 bp, 30 bp, 40 bp, 50 bp, 60 bp, 70 bp, 80 bp, 90 bp, 100 bp, 110 bp, 120 bp, 130 bp, 140 bp, 150 bp, 160 bp, 170 bp, 180 bp, 190 bp, 200 bp, 210 bp, 220 bp, 230 bp, 240 bp, 250 bp, 260 bp, 270 bp, 280 bp, 290 bp, 300 bp, 310 bp, 320 bp, 330 bp, 340 bp, 350 bp, 360 bp, 370 bp, 380 bp, 390 bp, 400 bp, 410 bp, 420 bp, 430 bp, 440 bp, 450 bp, 460 bp, 470 bp, 480 bp, 490 bp, 500 bp, 510 bp, 520 bp, 530 bp, 540 bp, 550 bp, 560 bp, 570 bp, 580 bp, 590 bp, 600 bp, 610 bp, 620 bp, 630 bp, 640 bp, 650 bp, 660 bp, 670 bp, 680 bp, 690 bp, 700 bp, 710 bp, 720 bp, 730 bp, 740 bp, 750 bp, 760 bp, 770 bp, 780 bp, 790 bp, 800 bp, 810 bp, 820 bp, 830 bp, 840 bp, 850 bp, 860 bp, 870 bp, 880 bp, 890 bp, 900 bp, 910 bp, 920 bp, 930 bp, 940 bp, 950 bp, 960 bp, 970 bp, 980 bp, 990 bp, 1000 bp, or more.

In some embodiments, the method of identifying at least one extrachromosomal circular DNA (eccDNA) in a sample comprising double-stranded DNA, such as those provided herein, further comprises the step of performing eccDNA enrichment. In some embodiments, the eccDNA enrichment comprises performing a size selection, such as those provided herein; and/or performing an exonuclease treatment, such as those provided herein.

In some embodiments, the methods provided herein are used to evaluate clastogenicity of a potential clastogen, such as but not limited to, a compound, a physical exposure, a biological agent, or a complex mixture and/or an environmental exposure. In some embodiments, a method of evaluating clastogenicity of a potential clastogen comprises obtaining double-stranded DNA from one or more cells exposed to the clastogen; performing duplex sequencing of the double-stranded DNA to obtain a plurality of sequence reads of the double-stranded DNA; identifying a subset of sequence reads from the plurality of sequence reads, wherein sequence reads of the subset of sequence reads each independently comprise a reference allele junction; from amongst the subset of sequence reads, distinguishing putative eccDNA sequence reads from chromosomal tandem duplication sequence reads; determining a profile of eccDNA according to the distinguished putative eccDNA sequence reads; and evaluating clastogenicity of the potential clastogen according to the determined profile of the eccDNA. In some embodiments, a method of evaluating clastogenicity of a potential clastogen comprises obtaining double-stranded DNA comprising putative extrachromosomal circular DNA (eccDNA) from one or more cells exposed to the clastogen; performing duplex sequencing of the double-stranded DNA to obtain a plurality of sequence reads of the double-stranded DNA; identifying a subset of sequence reads from the plurality of sequence reads, wherein sequence reads of the subset of sequence reads each independently comprise a reference allele junction; from amongst the subset of sequence reads, distinguishing putative eccDNA sequence reads from chromosomal tandem duplication sequence reads; determining a profile of eccDNA according to the distinguished putative eccDNA sequence reads; and evaluating clastogenicity of the potential clastogen according to the determined profile of the eccDNA.

In some embodiments, a method of evaluating clastogenicity of a potential clastogen comprises obtaining double-stranded DNA comprising putative extrachromosomal circular DNA (eccDNA) from one or more cells exposed to the clastogen; performing duplex sequencing of the double-stranded DNA to obtain a plurality of sequence reads of the double-stranded DNA; identifying a subset of sequence reads from the plurality of sequence reads, wherein sequence reads of the subset of sequence reads each independently comprise a reference allele junction; from amongst the subset of sequence reads, distinguishing putative eccDNA sequence reads from chromosomal tandem duplication sequence reads, wherein the distinguishing putative eccDNA sequence reads comprises the steps of: selecting a subset of D-A junction-containing consensus sequencing reads with allele length less than a threshold apparent insert size; inferring the fragment size of each read pair in the subset and comparing it to the threshold apparent insert size, one by one, or comparing the distribution of inferred fragment sizes to the distribution of insert sizes in the entire library; and comparing the inferred fragment size of any one consensus read pair to the allele; determining a profile of eccDNA according to the distinguished putative eccDNA sequence reads; and evaluating clastogenicity of the potential clastogen according to the determined profile of the eccDNA, wherein the evaluating clastogenicity of the potential clastogen further comprises the step of comparing the eccDNA profiles from one or more cells exposed to the clastogen to control or untreated samples from the same cohort.

In some embodiments, a method of evaluating clastogenicity of a potential clastogen comprises obtaining double-stranded DNA from one or more cells; performing duplex sequencing of the double-stranded DNA to obtain a plurality of sequence reads of the double-stranded DNA; identifying a subset of sequence reads from the plurality of sequence reads, wherein sequence reads of the subset of sequence reads each independently comprise a reference allele junction; from amongst the subset of sequence reads, distinguishing putative eccDNA sequence reads from chromosomal tandem duplication sequence reads, wherein the distinguishing putative eccDNA sequence reads comprises the steps of: selecting a subset of D-A junction-containing consensus sequencing reads with allele length less than a threshold apparent insert size; inferring the fragment size of each read pair in the subset and comparing it to the threshold apparent insert size, one by one, or comparing the distribution of inferred fragment sizes to the distribution of insert sizes in the entire library; and comparing the inferred fragment size of any one consensus read pair to the allele; evaluating clastogenicity of the potential clastogen according to the distinguishing putative eccDNA. In some embodiments, the one or more cells do not comprise eccDNA.

In some embodiments, the methods provided herein are used to evaluate genotoxicity of a compound. In some embodiments, the compound is a xenobiotic, such as those provided herein, a clastogen, such as those provided herein, or any mutagen. In some embodiments, a method of evaluating genotoxicity comprises obtaining double-stranded DNA comprising putative extrachromosomal circular DNA (eccDNA) from one or more cells exposed to a xenobiotic; performing duplex sequencing of the double-stranded DNA to obtain a plurality of sequence reads of the double-stranded DNA; identifying a subset of sequence reads from the plurality of sequence reads, wherein sequence reads of the subset of sequence reads each independently comprise a reference allele junction; from amongst the subset of sequence reads, distinguishing putative eccDNA sequence reads from chromosomal tandem duplication sequence reads; determining a profile of eccDNA according to the distinguished putative eccDNA sequence reads; and evaluating clastogenicity of the xenobiotic according to the determined profile of the eccDNA.

In some embodiments, the method of evaluating genotoxicity of a compound or exposure comprises the steps of a) evaluating clastogenicity comprising the steps of: obtaining double-stranded DNA from one or more cells exposed to a potential genotoxin; performing duplex sequencing of the double-stranded DNA to obtain a plurality of sequence reads of the double-stranded DNA; identifying a subset of sequence reads from the plurality of sequence reads, wherein sequence reads of the subset of sequence reads each independently comprise a reference allele junction; from amongst the subset of sequence reads, distinguishing putative eccDNA sequence reads from chromosomal tandem duplication sequence reads; determining a profile of eccDNA according to the distinguished putative eccDNA sequence reads; and evaluating clastogenicity of the potential genotoxin according to the determined profile of the eccDNA; and b) evaluating mutagenicity comprising the steps of: obtaining double-stranded DNA from one or more cells exposed to a mutagen; performing duplex sequencing of the double-stranded DNA to obtain a plurality of sequence reads of the double-stranded DNA; determining a mutation profile of the double-stranded DNA; and evaluating mutagenicity of the potential genotoxin according to the determined mutation profile of the double-stranded DNA.

In some embodiments, the method of evaluating genotoxicity of a compound or exposure comprises the steps of a) evaluating clastogenicity comprising the steps of: obtaining double-stranded DNA from one or more cells exposed to a potential genotoxin; performing duplex sequencing of the double-stranded DNA to obtain a plurality of sequence reads of the double-stranded DNA; identifying a subset of sequence reads from the plurality of sequence reads, wherein sequence reads of the subset of sequence reads each independently comprise a reference allele junction; from amongst the subset of sequence reads, distinguishing putative eccDNA sequence reads from chromosomal tandem duplication sequence reads; determining a profile of eccDNA according to the distinguished putative eccDNA sequence reads, wherein distinguishing putative eccDNA sequence reads comprises the steps of: selecting a subset of D-A junction-containing consensus sequencing reads with allele length less than a threshold insert size; inferring the fragment size of each read pair in the subset and comparing it to the threshold insert size, one by one, or comparing the distribution of inferred fragment sizes to the distribution of insert sizes in the entire library; and comparing the inferred fragment size of any one consensus read pair to the allele; and evaluating clastogenicity of the potential genotoxin according to the determined profile of the eccDNA; and b) evaluating mutagenicity comprising the steps of: obtaining double-stranded DNA from one or more cells exposed to a mutagen; performing duplex sequencing of the double-stranded DNA to obtain a plurality of sequence reads of the double-stranded DNA; determining a mutation profile of the double-stranded DNA; and evaluating mutagenicity of the potential genotoxin according to the determined mutation profile of the double-stranded DNA. In some embodiments, the potential genotoxin is any compound, physical exposure, environmental exposure, a biologic, or any other source capable of damaging the DNA.

In some embodiments, the methods provided herein are used for assessing cancer risk in a sample. In some embodiments, a method of assessing cancer risk in a sample comprises obtaining double-stranded DNA comprising putative extrachromosomal circular DNA (eccDNA) from the sample; performing duplex sequencing of the double-stranded DNA to obtain a plurality of sequence reads of the double-stranded DNA; identifying a subset of sequence reads from the plurality of sequence reads, wherein sequence reads of the subset of sequence reads each independently comprise a reference allele junction; from amongst the subset of sequence reads, distinguishing putative eccDNA sequence reads from chromosomal tandem duplication sequence reads; determining a profile of eccDNA according to the distinguished putative eccDNA sequence reads; and evaluating cancer risk of the sample according to the determined profile of the eccDNA.

In some embodiments, a method of assessing cancer risk in a sample comprises obtaining double-stranded DNA comprising putative extrachromosomal circular DNA (eccDNA) from the sample; performing duplex sequencing of the double-stranded DNA to obtain a plurality of sequence reads of the double-stranded DNA; identifying a subset of sequence reads from the plurality of sequence reads, wherein sequence reads of the subset of sequence reads each independently comprise a reference allele junction; from amongst the subset of sequence reads, distinguishing putative eccDNA sequence reads from chromosomal tandem duplication sequence reads, wherein distinguishing putative eccDNA sequence reads comprises the steps of: selecting a subset of D-A junction-containing consensus sequencing reads with allele length less than a threshold insert size; inferring the fragment size of each read pair in the subset and comparing it to the threshold insert size, one by one, or comparing the distribution of inferred fragment sizes to the distribution of insert sizes in the entire library; and comparing the inferred fragment size of any one consensus read pair to the allele; determining a profile of eccDNA according to the distinguished putative eccDNA sequence reads; and evaluating cancer risk of the sample according to the determined profile of the eccDNA. In some embodiments, the sample is a cancerous sample, or a healthy sample. In some embodiments, the evaluating cancer risk of the sample according to the determined profile of the eccDNA further comprises the step of comparing the eccDNA profiles to known eccDNA profiles.

In some embodiments, a cell, a tissue, or an organoid is exposed to one or more compounds. In some embodiments, the cell is an eukaryotic cell. In some embodiments, the cell is an animal cell. In some embodiments, the cell is a human cell. In some embodiments, the cell is any animal cell. In some embodiments, the cell is any human cell. In some embodiments, the cell is any cell. In some embodiments, the cell is a muscle cell. In some embodiments, the cell is a nerve cell. In some embodiments, the cell is a blood cell. In some embodiments, the cell is a connective tissue cell. In some embodiments, the cell is an epithelial cell. In some embodiments, the cell is a reproductive cell. In some embodiments, the cell is an endocrine cell. In some embodiments, the cell is an immune system cell. In some embodiments, the cell is a stem cell. In some embodiments, the cell is a healthy cell. In some embodiments, the cell is a cancer cell. In some embodiments, the tissue is an animal tissue. In some embodiments, the tissue is a human tissue. In some embodiments, the tissue is any animal tissue. In some embodiments, the tissue is any human tissue. In some embodiments, the tissue is any tissue. In some embodiments, the tissue is epithelial tissue. In some embodiments, the tissue is connective tissue. In some embodiments, the tissue is muscle tissue. In some embodiments, the tissue is nervous tissue. In some embodiments, the organoid is an animal organoid. In some embodiments, the organoid is a human organoid. In some embodiments, the organoid is any organoid.

As provided herein, eccDNAs are DNA fragments that exist outside of linear chromosomes and are often by-products of DNA breakage and repair. eccDNAs may contain both coding and non-coding sequences and have been found in both normal and cancerous cells. “Clastogenicity”, as used herein, refers to the property of certain agents to induce chromosomal breakage. Without wishing to be bound by a particular theory, clastogenic events may contribute to the formation of eccDNAs. Chromosomal breakage, a hallmark of clastogenic events, can give rise to these circular DNAs, adding another layer of complexity to the genome. Both eccDNAs and clastogenicity serve as markers of genomic instability, making them relevant in the context of diseases like cancer. For example, elevated levels of eccDNAs and clastogenic events are often observed in malignancies and could potentially serve as diagnostic or prognostic markers. In some embodiments, clastogens or potential clastogens may be agents, such as compounds, variants thereof, or derivatives thereof, that induce chromosomal breaks, leading to mutations. Non-limiting examples include chemical compounds such as Ethyl Methanesulfonate (EMS), Methyl Methanesulfonate (MMS), Ethylene Oxide, Acetaldehyde, Formaldehyde, Benzene, Vinyl Chloride, Cadmium, Nickel Compounds, Chromium(VI) Compounds, Lead Compounds, and Arsenic Compounds, Cyclophosphamide, Nitrogen Mustard, Melphalan, Chlorambucil, Colchicine, 5-Fluorouracil, Hydroquinone, Adriamycin, Actinomycin D, Camptothecin, Etoposide, Cisplatin, Azathioprine, 6-Mercaptopurine, Methotrexate, Aflatoxins, Thalidomide, Hydroxyurea, Bleomycin, Naphthalene, and 2-Acetylaminofluorene (2-AAF), a variant thereof, or a derivative thereof. Non-limiting examples of physical clastogens include X-Rays, Gamma Rays, and Ultraviolet Radiation. Non-limiting examples of biological agents include certain Oncoviruses like HPV and Epstein-Barr Virus, and bacteria like Helicobacter pylori. Non-limiting examples of complex mixtures and environmental exposures include Tobacco Smoke, Polychlorinated Biphenyls (PCBs), Asbestos, Diesel Exhaust, Crude Oil, and pesticides like Atrazine and Paraquat.

In some embodiments, the compound is a xenobiotic. In some embodiments, the xenobiotic is selected from any one of environmental pollutants, hydrocarbons, food additives, oil mixtures, pesticides, other xenobiotics, synthetic polymers, carcinogens, drugs, antioxidants, and any combination thereof.

In some embodiments, the method comprises, prior to performing or having performed duplex sequencing, as provided herein, exposing one or more cells, tissues, or organoids, to a compound; and obtaining the eccDNA from the one or more cells, tissues, or organoids. In some embodiments, the method further comprises evaluating clastogenicity of the compound based on the determined profile of the eccDNA. In some embodiments, the profile of the eccDNA comprises any one, or any combination, of quantity of the eccDNA, frequency of the eccDNA, quality of the eccDNA, size of the eccDNA or a fragment thereof, genomic location of the eccDNA, or any other characteristic of the eccDNA. In some embodiments, the profile of the eccDNA comprises quantity of the eccDNA. In some embodiments, the profile of the eccDNA comprises frequency of the eccDNA. In some embodiments, the profile of the eccDNA comprises quality of the eccDNA. In some embodiments, the profile of the eccDNA comprises size of the eccDNA or a fragment thereof. In some embodiments, the profile of the eccDNA comprises genomic location of the eccDNA. In some embodiments, the profile of the eccDNA comprises any characteristic of the eccDNA. In some embodiments, the potential clastogen is a direct clastogen. In some embodiments, the potential clastogen is an indirect clastogen.

### VI. COMPUTER IMPLEMENTATION

The methods disclosed herein, are, in some embodiments, performed on one or more computers. In various embodiments, the eccDNA detection system 130 described in FIG. 1A can be embodied as one or more computers. For example, the identifying or having identified the eccDNA using the plurality of sequence reads of the duplex sequencing, and database storage can be implemented in hardware or software, or a combination of both. In one embodiment, a machine-readable storage medium is provided, the medium comprising a data storage material encoded with machine readable data which, when using a machine programmed with instructions for using said data, is capable of displaying any of the datasets and execution and results of an identification model of this disclosure. Such data can be used for a variety of purposes, such as patient monitoring, treatment considerations, and the like. The methods can be implemented in computer programs executing on programmable computers, comprising a processor, a data storage system (including volatile and non-volatile memory and/or storage elements), a graphics adapter, a pointing device, a network adapter, at least one input device, and at least one output device. Program code may be applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The data and databases thereof can be provided in a variety of media to facilitate their use. “Media” refers to a manufacture that contains the signature pattern information. The databases can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g., word processing text file, database format, etc.

### VII. KITS

The present disclosure also provides kits for performing the herein disclosed methods, e.g., methods of detecting putative eccDNA molecules in a biological sample, methods of treating a disease or other medical condition in a mammalian, e.g., human, subject, and methods of preparing a sequencing library for the detection of putative eccDNA molecules in a biological sample. Such kits may comprise, e.g., one or more reagents for performing any of the herein-disclosed methods, e.g., sequencing adapters, exonucleases, ligases, one or more physical implements for performing the methods, e.g., reaction vessels, columns, supports, or containers, and/or instructions for performing any of the herein-disclosed methods, e.g., printed instructions and/or instructions provided on electronic media.

### VIII. ADDITIONAL EMBODIMENTS

In some embodiments, a method of detecting candidate extrachromosomal circular DNA (eccDNA) molecules in a biological sample comprises: (a) providing a sequencing library comprising a plurality of double-stranded DNA fragments obtained from the sample; (b) obtaining error-corrected sequences for double-stranded DNA fragments in the library; (c) detecting possible insertions in the double-stranded DNA fragments by aligning a plurality of the error-corrected sequences with a reference genome; (d) detecting putative eccDNA breakpoints in one or more of the fragments in which a possible insertion has been detected, wherein a putative eccDNA breakpoint comprises a sequence B located upstream of a sequence A, wherein: (i) sequence A is present upstream of sequence B in the reference genome; (ii) the first nucleotide of sequence A is located distance of Y nucleotides upstream of the last nucleotide of sequence B in the reference genome; and (iii) the last nucleotide of sequence B is located approximately immediately upstream of the first nucleotide of sequence A in the putative eccDNA breakpoint: (e) detecting putative eccDNA molecules among the fragments comprising a putative eccDNA breakpoint, wherein a putative eccDNA molecule is a fragment comprising a putative eccDNA breakpoint that is not excluded by any one or more of steps (i), (ii), or (iii), the steps comprising: (i) comparing the length of the fragment to the distance Y nucleotides, wherein a determination that the fragment is longer than Y nucleotides indicates that the fragment is not a putative eccDNA molecule; (ii) determining whether the error-corrected sequence for the fragment comprises a duplication of any subsequence comprised within the region from sequence A to sequence B in the reference genome, wherein a detection of a duplication indicates that the fragment is not a putative eccDNA molecule; and/or (iii) determining whether a sequence located upstream of sequence A in the reference genome is present upstream of sequence B in the error-corrected sequence for the fragment, or whether a sequence located downstream of sequence B in the reference genome is present downstream of sequence A in the error-corrected sequence for the fragment, wherein a detection that a sequence located upstream of sequence A or downstream of sequence B in the reference genome is present upstream of sequence B or downstream of sequence A, respectively, in the error-corrected sequence indicates that the fragment is not a putative eccDNA molecule.

In some embodiments, the error-corrected sequences obtained in (b) are obtained using consensus sequencing. In some embodiments, the consensus sequencing is duplex sequencing (DS). In some embodiments, the consensus sequencing is single-stranded consensus sequencing (SSCS) or a combination of DS and SSCS. In some embodiments, the biological sample is selected from the group consisting of a sperm sample, semen sample, prostatic fluid sample, testicular biopsy sample, spermatogonia sample, germ cell sample, gamete sample, swab, lavage, aspirate, biopsy, tissue sample, tumor sample, preneoplastic sample, liquid biopsy, hyperplasia sample, hypertrophy sample, dysplastic sample, urine sample, CSF sample, any other body fluid sample, autopsy sample, necropsy sample, surgical sample, model organism sample, plasma sample, serum sample, gastric sample, bone marrow sample, stool sample, brushing sample, bile sample, pancreatic fluid sample, synovial fluid sample, sputum sample, mucus sample, vitreous sample, forensic sample, environmental sample, bacterial sample, fungal sample, mammalian sample, human sample, and diagnostic sample. In some embodiments, the biological sample comprises potential cancer cells or potentially cancer-derived nucleic acids. In some embodiments, the biological sample comprises cell-free DNA. In some embodiments, the biological sample comprises cells that have been exposed to a potentially toxic agent. In some embodiments, the potentially toxic agent is a potentially clastogenic, aneugenic, mutagenic, and/or teratogenic agent. In some embodiments, the presence and/or character of eccDNA molecules in the sample is used to identify a disease state or a physiological state. In some embodiments, the disease state or physiological state is selected from the group consisting of cancer, inflammation, autoimmunity, infection, organ transplant rejection, stem cell transplant rejection, therapeutic cell rejection, therapeutic cell response, immunotherapy response, pregnancy, pre-eclampsia, radiation exposure, sun exposure, drug exposure, and hypersensitivity. In some embodiments, the double-stranded DNA fragments were obtained by enzymatic fragmentation. In some embodiments, the average length of the double-stranded DNA fragments in the library is between about 100 bp and 1000 bp. In some embodiments, the average length of the double-stranded DNA fragments in the library is greater than about 1000 bp, 2000 bp, 3000 bp, 4000 bp, 5000 bp, 6000 bp, 7000 bp, 8000 bp, 9000 bp, or 10,000 bp. In some embodiments, the length of the putative eccDNA molecule is between about 100 and 1000 nucleotides. In some embodiments, the length of the putative eccDNA molecule is less than about 500 nucleotides. In some embodiments, the length of the putative eccDNA molecule is greater than about 1000 bp, 2000 bp, 3000 bp, 4000 bp, 5000 bp, 6000 bp, 7000 bp, 8000 bp, 9000 bp, or 10,000 bp. In some embodiments, the length of the putative eccDNA molecule is greater than about 100 kb, 200 kb, 300 kb, 400 kb, 500 kb, 600 kb, 700 kb, 800 kb, 900 kb, 1 Mb, 2 Mb, or 3 Mb. In some embodiments, the putative eccDNA molecule comprises a gene. In some embodiments, the putative eccDNA molecule comprises an origin of replication. In some embodiments, the length of the putative eccDNA molecule is approximately equal to the distance Y nucleotides. In some embodiments, the length of the putative eccDNA molecule is exactly equal to the distance Y nucleotides. In some embodiments, the length of the putative eccDNA molecule is less than about 50%, 60%, 70%, 80%, 90%, or more of the average length of the DNA fragments in the library. In some embodiments, the error-corrected sequences obtained in (b) are specific to a single genomic region. In some embodiments, the error-corrected sequences obtained in (b) are specific to from about 1 to about 30 individual genomic loci. In some embodiments, the method is performed with or without an enrichment step to increase the proportion of double-stranded circular DNA molecules among all double-stranded nucleic acids in the sample, and further comprising: comparing the frequencies in the library of possible insertions as detected in step (c), of putative eccDNA breakpoints as detected in step (d), and/or of putative eccDNA molecules as detected in step (e), obtained with the method performed with or without the enrichment step. In some embodiments, the enrichment step comprises selectively eliminating double-stranded linear DNA molecules from the sample. In some embodiments, the double-stranded linear DNA molecules are selectively eliminated by treating the sample with one or more exonucleases. In some embodiments, the enrichment step comprises selectively isolating double-stranded circular DNA molecules from the sample. In some embodiments, the double-stranded circular DNA molecules are selectively isolated using electrophoresis, column filtration, density gradient centrifugation, selective extraction, and/or using a DNA binding protein that will differentially bind to or remain bound to double-stranded circular DNA molecules as compared to double-stranded linear DNA molecules. In some embodiments, the DNA binding protein is a helicase. In some embodiments, the ratio of the number of putative eccDNA molecules detected in step (e) to the number of error-corrected sequences obtained in step (b), to the number of possible insertions detected in step (c), or to the number of putative eccDNA breakpoints detected in step (d), is higher when the method is performed with the enrichment step than without the enrichment step. In some embodiments, the frequency of possible insertions as detected in step (c) with the enrichment step is less than about 90%, 80%, 70%, 60%, 50%, 40%, 30%, 20%, or 10% of the frequency of possible insertions as detected in step (c) without the enrichment step. In some embodiments, the frequency of putative eccDNA breakpoints as detected in step (d) with the enrichment step is less than about 90%, 80%, 70%, 60%, 50%, 40%, 30%, 20%, or 10% of the frequency of putative eccDNA breakpoints as detected in step (d) without the enrichment step. In some embodiments, the frequency of putative eccDNA molecules as detected in step (e) with the enrichment step is at least about 10%, 20%, 30%, 40%, 50%, 60%, 70%, 80%, 90%, or more of the frequency of putative eccDNA molecules as detected in step (e) without the enrichment step. In some embodiments, the method further comprises performing a calculation of a probability that a putative eccDNA molecule identified in step (e) is a genuine eccDNA molecule. In some embodiments, the calculation is based in part on any one or more of the frequencies, ratios, or percentages described herein. In some embodiments, the calculation in based in part on a relationship between the length of the putative eccDNA molecule to the average length of double-stranded DNA fragments in the library, wherein a lower length of the putative eccDNA molecule relative to the average length of double-stranded DNA fragments in the library indicates a higher probability that the putative eccDNA molecule is a genuine eccDNA molecule. In some embodiments, the calculation is based in part on an observation that the length of the putative eccDNA molecule is approximately or exactly equal to the distance Y nucleotides, wherein the observation indicates a higher probability that the putative eccDNA molecule is a genuine eccDNA molecule. In some embodiments, any one or more of steps (a)-(e), a determination of any one or more of the frequencies, ratios, or percentages, or the calculation disclosed herein, is performed on a computer. In some embodiments, any one or more of steps (a)-(e), a determination of any one or more of the frequencies, ratios, or percentages, or the calculation disclosed herein, is performed in the cloud. In some embodiments, a computer-based system for performing any of the methods provided herein is provided.

In some embodiments, a method of treating a disease or other medical condition in a mammalian subject comprises: (i) performing a method disclosed herein on a biological sample obtained from the subject; (ii) identifying one or more putative eccDNA molecules in the sample that are indicative of the disease or of a physiological state associated with the medical condition; and (iii) treating the subject for the disease or medical condition.

In some embodiments, a method of preparing a sequencing library for the detection of putative extrachromosomal circular DNA (eccDNA) molecules in a biological sample comprises: (a) providing a biological sample comprising double-stranded DNA; (b) preparing a first, unenriched portion of the biological sample and a second, enriched portion of the biological sample that is enriched for double-stranded circular DNA molecules, wherein the second portion is prepared by selectively eliminating linear double-stranded DNA molecules and/or selectively isolating double-stranded circular DNA molecules from the sample; (c) fragmenting double-stranded DNA molecules in the first portion of the biological sample to produce a population of unenriched double-stranded DNA fragments, and enzymatically fragmenting double-stranded DNA molecules in the second portion of the biological sample to produce a population of enriched double-stranded DNA fragments; (d) ligating sequencing adapters to a plurality of the unenriched double-stranded DNA fragments to produce an unenriched sequencing library; and (e) ligating sequencing adapters to a plurality of the enriched double-stranded DNA fragments to produce an enriched sequencing library. In some embodiments, the biological sample is selected from the group consisting of a sperm sample, semen sample, prostatic fluid sample, testicular biopsy sample, spermatogonia sample, germ cell sample, gamete sample, swab, lavage, aspirate, biopsy, tissue sample, tumor sample, preneoplastic sample, liquid biopsy, hyperplasia sample, hypertrophy sample, dysplastic sample, urine sample, CSF sample, any other body fluid sample, autopsy sample, necropsy sample, surgical sample, model organism sample, plasma sample, serum sample, gastric sample, bone marrow sample, stool sample, brushing sample, bile sample, pancreatic fluid sample, synovial fluid sample, sputum sample, mucus sample, vitreous sample, forensic sample, environmental sample, bacterial sample, fungal sample, mammalian sample, human sample, and diagnostic sample. In some embodiments, the biological sample comprises potential cancer cells or potentially cancer-derived nucleic acids. In some embodiments, the biological sample comprises cell-free DNA. In some embodiments, the biological sample comprises cells that have been exposed to a potentially toxic agent. In some embodiments, the potentially toxic agent is a potentially clastogenic, aneugenic, mutagenic, and/or teratogenic agent. In some embodiments, the fragmentation is enzymatic fragmentation. In some embodiments, the second sample is enriched by treating the sample with one or more exonucleases. In some embodiments, the one or more exonucleases comprise exonuclease I, exonuclease T, exonuclease VII, exonuclease III, T7 exonuclease, exonuclease V (RecBCD), exonuclease VIII, lambda exonuclease, or T5 exonuclease. In some embodiments, the method further comprises treating a portion of the second sample with one or more endonucleases prior to treating the sample with one or more exonucleases, and comparing the putative eccDNAs obtained in the presence or absence of treatment with the one or more endonucleases. In some embodiments, the second sample is enriched by selectively isolating double-stranded circular DNA molecules from the sample. In some embodiments, the double-stranded circular DNA molecules are selectively isolated using electrophoresis, column filtration, density gradient centrifugation, selective extraction, and/or using a DNA binding protein that will differentially bind to or remain bound to double-stranded circular DNA molecules as compared to double-stranded linear DNA molecules. In some embodiments, the DNA binding protein is a helicase. In some embodiments, the biological sample is treated with DTT prior to step (b), (c) or (d). In some embodiments, the method further comprises: preparing a third and a fourth portion of the biological sample by removing sub-portions of the first, unenriched, and the second, enriched, portions prepared in step (b), respectively; treating a fraction of the third and a fraction of the fourth portions with a reagent that induces breaks in double-stranded circular DNA molecules at sites of DNA damage, and leaving a fraction of the third and the fourth portions untreated; ligating sequencing adapters to the treated and untreated fractions of the third and the fourth portions. In some embodiments, the reagent is FPG (formamidopyrimidine [fapy]-DNA glycosylase) or UDG (Uracil-DNA Glycosylase) with endonuclease VIII. In some embodiments, the sequencing adapters are duplex sequencing adapters. In some embodiments, the sequencing adapters comprise a Y shape. In some embodiments, the sequencing adapters are hairpin adapters. In some embodiments, a sequencing library prepared using any of the methods provided herein, is provided. In some embodiments, a kit for performing any of the methods provided herein, is provided.

Further aspects of the invention are also described in the following numbered clauses:

1. A method of identifying at least one extrachromosomal circular DNA (eccDNA) in a sample comprising double-stranded DNA, the method comprising:
performing or having performed duplex sequencing on the sample,
identifying or having identified the eccDNA from the plurality of sequence reads of the duplex sequencing.

2. Use of duplex sequencing to identify at least one extrachromosomal circular DNA (eccDNA) in a sample comprising double-stranded DNA.

The definitions, descriptions and further features as applied to the methods in the application apply equally to the above use clause.

3. The method of clause 1 or the use of clause 2, wherein the duplex sequencing comprises:
a) tagging the double-stranded DNA fragments in the sample by ligating adaptors to the ends of the DNA, wherein each strand within each DNA fragment is tagged to: i) add a unique molecule identifier (UMI) which labels the strands as being from one DNA molecule; and ii) label each strand with a strand differentiator (SDE) to allow the first strand to be distinguished from the second strand within the one DNA molecule;
b) amplifying the tagged DNA; and
c) sequencing the tagged amplicons, wherein the reads from each strand can be identified as being from one DNA molecule due to the UMI labels and reads from the first strand of the DNA molecule can be differentiated from reads from the second strand of the same DNA molecule due to the SDE labels.

UMI labels are also known as SMI (single molecule identifier) labels in the art.

4. The method or use of clause 3, further comprising:
d) for the reads from one DNA molecule, comparing the first strand sequence reads and the second strand sequence reads.

When compared, at any position where the first strand reads and the second strand reads do not agree, this nucleotide can be (is) discounted.
5. The method or use of any of the preceding clauses, wherein duplex sequencing comprises single-strand consensus sequencing (SSCS) and/or duplex consensus sequencing (DCS).

The clauses 3-5 apply equally to other aspects of the invention, for example as described in claim 38, and at paragraph 7. Additionally, claims 2-37 may be dependent from clause 1 or any of clauses 3-5.

### IX. EXAMPLES

The following section provides some non-limiting examples of methods for preparing sequencing libraries and detecting putative eccDNAs using duplex sequencing.

### Example 1. Preparation of Sequencing Libraries from Sperm and Blood Samples, and Analysis of Sequencing Data

### PRJ00150 Prep

Samples: paired blood and sperm from 6 young men. DNA from blood was isolated with a Qiagen kit following manufacturer's instructions, and sperm were isolated according to standard protocol (including high DTT & bead-beating). Otherwise, the samples were processed according to duplex-sequencing standard protocols for Covaris and enzymatic fragmentation, respectively.

Data were processed through the TwinStrand standard internal pipeline.

Putative eccDNAs were identified as follows:

Mut files were pre-processed in dsreporter and then filtered for variation_type==“indel” & length(alt)>length(ref) to identify all variant calls consistent with an insertion. From here on, the alt allele will be called α.

Histograms of length(α) were plotted for various combinations of samples and treatments, revealing a striking periodicity for most samples/groupings.

### PRJ00178 Prep

Samples: Sperm DNA from PRJ00150, pooled across individuals. All other samples were TS human devDNA (DNA extracted from blood of a young, healthy human blood donor).

Sample pre-processing: None (processed according to standard protocol).

devDNA was exposed to 10, 150, or 600 mM of DTT (plus IDTE and water controls) at 37 degrees C. overnight and then cleaned with 1.6×SPRI beads, to simulate DTT-related damage during sperm DNA extraction.

Sperm and devDNA were treated with 1×LCM (FPG and UDG) for 1 hour at 37 degrees C. and then cleaned with 1.8×SPRI beads, to try to fix any pre-existing DNA damage that could cause artifactual variant calls.

Samples were otherwise processed according to Duplex-sequencing standard protocols for Covaris and enzymatic fragmentation, respectively.

Data were processed through the standard TwinStrand internal pipeline.

Putative eccDNAs were identified as follows:

Mut files were pre-processed in dsreporter and then filtered for variation_type==“indel” & length(alt)>length(ref) to identify all variant calls consistent with an insertion. From here on, the alt allele will be called α.

Histograms of length(α) were plotted for various combinations of samples and treatments. These histograms revealed a striking periodicity for most samples/groupings.

matchedPattern function from BSgenome.Hsapiens.UCSC.hg38 package was used to identify location(s) of a within the chromosome containing the variant call, allowing 1 mismatch per 50 nt.

If an alt allele (a) matched the chromosome and position of the variant call, this was considered to imply the presence of a BA junction, which is consistent with either a chromosomal tandem duplication or an eccDNA. They were considered putative eccDNAs for the reasons listed below.

Reads supporting representative putative eccDNAs were identified and examined in IGV.

### Customer Mouse Tumor-Normal Samples

DNA was extracted with a Qiagen kit for all sample types. Duplex sequencing was performed according to the kit protocol, and data were analyzed using TS pipeline on DNANexus.

Additional data processing: Mut files were pre-processed in dsreporter and then filtered for variation_type==“indel” & length(alt)>length(ref) to identify all variant calls consistent with an insertion. From here on, the alt allele will be called α.

Histograms of length(α) were plotted for various combinations of samples and treatments, revealing a striking periodicity for most samples/groupings.

Evidence that Putative eccDNAs were not Artifacts Caused by DNA Damage During Sperm DNA Isolation:

First, pre-treating sperm DNA with LCM to remove damaged molecules did not reduce the number of eccDNA candidates identified (compared to matched controls). Further, pre-treating devDNA with high levels of DTT did not increase the number of eccDNA candidates identified (compared to matched controls).

Evidence that Putative eccDNAs are (Primarily) not Chromosomal TDs:

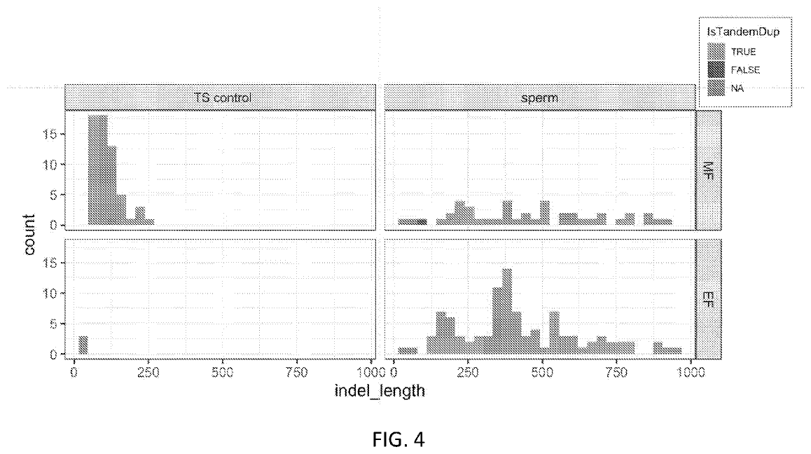

First, the size distribution of a was similar to reports for eccDNAs (specifically microDNAs), and the periodicity corresponds to the length of DNA around histone(s). Chromosomal TDs in this size range (<1 kb) are likely to occur during DNA replication or DNA repair, when normal chromatin structure is disrupted and thus not expected to influence the outcomes. This size distribution pattern is consistent with that of known eccDNA (see for example, Dillon, L. et al, Cell Reports, 2015; Mehana, P. et al. PloS One, 2017). Furthermore, this size distribution is independent of fragmentation method (also seen with mechanically sheared libraries, see FIG. 4), further indicating the biological relevance of these indel calls.

Further, no examples were found where the insert size of the supporting consensus read(s) was larger than a, even for a smaller than the median insert size of the library where this would be expected to happen by chance for chromosomal TDs.

In addition, no examples were found where the consensus read(s) supporting the indel call contained more than one copy of any part of a or any sequencing originating from outside of a in the reference genome.

Furthermore, as shown for a particular example in FIGS. 5A-5D, some of the indel calls had lengths identical to the length of two sections having an apparent duplication linked by a novel junction. This bolsters the likelihood that such indel calls are due to the presence of circular genomically derived DNAs present in the original sample, which had been cleaved during sample prep in a single cleavage event. The enzymatic fragmentation method cuts DNA randomly, as evidence by the many other fragments in the library aligning to the same reference region but having a variety different cut sites/fragment ends. Thus, the likelihood for a tandem duplication event, of an enzyme cutting at the same exact breakpoint for both copies of the duplication is extremely low.

### Example 2. Method for Validating the Identification of Putative eccDNAs

### Primary Probative Experiment

Identify sample types with high observed or predicted putative eccDNA counts, including:
sample types where we have already identified putative eccDNA (e.g., human sperm, mouse GI tumors);
reanalyze existing DS data to identify other samples/sample types with high putative eccDNA counts;
identify sample types that have previously been shown to have high eccDNA burdens via other methods.

Ideally, genomic DNA is isolated from all samples of interest using a single, gentle DNA isolation method (e.g. Qiagen Blood & Tissue kit). Some sample types, such as sperm, may require specialize extraction methods but care is taken to isolate high quality DNA with minimal extraction-related damage.

For each sample, 2 matched DS libraries are prepared as follows:

Prepare one library according to standard protocol with ~500 ng of total genomic DNA as input and using the species-matched mutagenesis panel for hybrid capture.

For the second library, pretreat genomic DNA with Exonuclease V (RecBCD) to enrich for circular DNA as follows:

Spike in a bacterial plasmid at a 1:1 molar ratio (1 plasmid per GE) to genomic DNA to use as a control to monitor enrichment of circular DNA.

Add 50 U of Exonuclease V (NEB) to 5 ug of genomic DNA with plasmid spike-in and incubate at 37 degrees C. for 2 hr.

Inactivate Exo V by adding EDTA to a final concentration of 11 mM and incubating at 65 degrees C. for 30 min.

Purify remaining DNA by performing a 1.5×SPRI clean-up and quantify sample as follows:

Measure concentration with Qubit HS DNA kit, target >10-fold decrease in total DNA yield (relative to pre-digestion).

If DNA concentration is >0.5 ng/ul, run an Agilent Genomic DNA ScreenTape to confirm removal of high molecular weight chromosomal DNA.

Perform qPCR with primers targeting 3 randomly selected chromosomal regions and 1 plasmid region to confirm enrichment of circular DNA, target >100-fold depletion of at least one of the 3 chromosomal regions relative to the plasmid region (compared to pre-digestion sample).

If depletion goals (>10× by mass, >100× by qPCR) are not initially reached, Exo V treatment protocol is adjusted (e.g., double enzyme units and incubation time). If depletion is still inadequate, the amount of depletion is noted and libraries are still prepared as described below.

Prepare libraries with Exo V-digested DNA as follows:

If >1 ng of DNA remains after digestion, prepare DS libraries with 1 ng of input according to standard procedure with increased numbers of cycles at each PCR step (to be determined empirically).

If <1 ng of DNA remains after digestion, prepare libraries by mixing a known quantity of Exo V-digested DNA with bacterial plasmid DNA to act as carrier DNA during library construction (through the first PCR step). Note that bacterial plasmid DNA should be effectively removed during the hybrid capture steps of DS protocol.

Sequence and analyze DS libraries according to standard procedure, with addition of proposed eccDNA candidate identification pipeline. Additional details below:

If necessary, downsample one or both DS libraries in each sample pair (+/−Exo V) to have same PTFS before proceeding with eccDNA candidate identification.

Calculate eccDNA candidate frequency as a number of eccDNA candidates per informative duplex base in each library.

Expected results, which are probative for eccDNA candidate identification pipeline:

eccDNA candidate frequency (as described above) should be significantly higher in Exo V-treated libraries than in matched control libraries, with expected magnitude of change dependent on the amount of depletion accomplished by Exo V treatment (estimated through DNA yield and qPCR quantification, as described above)

Identification of identical eccDNA candidates in both libraries in sample pair (+/−Exo V) would be very strong evidence that those candidates are true circles; however, failure to identify identical eccDNA candidates across library pairs does not refute their identity as eccDNA. If a single copy of the eccDNA exists in the biological sample, it will get randomly partitioned into one or the other library and thus only be identified in 1 (or 0) library. Similarly, eccDNA that exists at very low copy number in the biological sample may not be detected in both libraries due to simple sampling statistics.

Similar size distribution profile of eccDNA candidates across both library types (+/−Exo V) supports validity of eccDNA calls in unmodified libraries

If no substantial decrease in eccDNA frequency in Exo V-treated libraries is observed as compared to matched controls (suggesting that most candidates are not true eccDNAs) or if a decrease is observed that is of a substantially lower magnitude than expected based on quantification of chromosomal DNA depletion in Exo V-treated libraries (suggesting a mix of true and false-positive eccDNA calls); of

If individual eccDNA candidates with support of more than 10 duplex consensuses (alt allele count >10) are observed in non-Exo-treated libraries that are not detected in Exo-treated libraries.

As an alternative to, or in addition to, Exo V-dependent depletion of linear chromosomal DNA, pairs of libraries could be prepped from DNA extracted from a single sample using 2 distinct methods, one for total chromosomal DNA (i.e. Qiagen Blood and Tissue kit) and one for plasmid isolation.

### Example 3. eccDNA Discovery in Human Sperm

The following analysis was conducted using samples and data provided in Example 1. In brief, human sperm DNA was obtained from 6 healthy young donors. Duplex sequencing (DS) libraries were prepared with 500 ng DNA input. DS libraries were prepared with 500 ng DNA input. The TwinStrand® DuplexSeq™ Human Mutagenesis Assay panel (20×2.4 kb targets across the genome) was used for hybrid capture. Because the distribution of allele lengths for large insertions was reminiscent of small extrachromosomal circular DNA (eccDNA) or microDNA, these variant calls were characterized further. First, the variant positions and alternate allele sequences were analyzed to determine whether they supported the type of junction expected for either circular DNA or chromosomal tandem duplications (junction fusing end and beginning of allele ABCD, subsequently referred to as a D-A junction). To identify D-A junctions (FIG. 6A-6C, and FIG. 7A-7C), the alternate allele sequences were written to a FASTA file, which was used as input for the matchPattern function from Biostrings (Bioconductor package) with the reference genome sequence from BSgenome.Hsapiens. UCSC.hg38 (Bioconductor package), allowing 1 mismatch per 50 bp. The search was performed on a per-chromosome basis, only finding matches on the chromosome of the variant call, and yielded 0 or 1 match per sequence. Variants were considered to support a D-A junction if matchPattern returned a match located exactly 1 bp downstream of the variant position in the original mut file.

For fragment length analysis, duplex consensus reads supporting variant calls were visualized in IGV. First, the presence of a D-A junction was confirmed by inspecting supplementary alignments and/or BLAT-searching any soft-clipped sequences. For all manually inspected events (read pairs with a D-A junction), it was determined that all 5′ soft-clipped bases aligned at the other end of the allele, supporting the D-A junction, and any 3′ soft-clipped bases represented read-through into the DS adapter (only present for fragments <142 bp in length). Most consensus read pairs containing D-A junctions aligned in discordant pairs, with one or both consensus reads having a substantial soft-clipped region with a supplementary alignment to the other end of the allele (spanning the D-A junction). For these read pairs, physical DNA fragment length was inferred by considering a pseudo-pair of reads made by the primary alignment of one read in the pair and the supplementary alignment of the other read in the pair and taking the difference between the right-most end of the reverse-aligned read and the left-most end of the forward-aligned read, including any 5′ soft-clipped bases. A few D-A junction-containing consensus read pairs aligned concordantly, in which case, the fragment length was inferred to be the computed insert size plus any 5′ soft-clipped bases. The distance between the 5′ ends of outward-facing read pairs (either primary-primary, or primary-supplementary) was noted based on visualization and confirmed to equal the allele length minus the inferred fragment length, except for the one confirmed chromosomal TD identified. To ensure a reasonable probability of detecting duplicated sequence if it was present, the subset of apparent insertion variant calls with allele length less than the median insert size for all sperm DS libraries (233 bp) was selected for systematic manual inspection. Considering the null hypothesis to be that all D-A junctions arose from chromosomal TDs, we expected that the length distribution of fragments supporting this subset of variant calls would be similar to the insert size distribution of the whole libraries, meaning that about half of fragments would be shorter than 233 bp, and the other half would be longer and that more than half of the fragments would be expected to include duplicated ABCD sequence (any fragment longer than its respective allele length). A binomial p-value was computed to test if the observed results were significantly different from the null hypothesis.

Most apparent large insertions were called based on split alignment of a duplex consensus read to the end and beginning of a reference allele (FIG. 6A i-iii). Given a reference allele, ABCD, this type of alignment shows a junction that joins the end of the reference allele (D) to the beginning of the reference allele (A) and which was referred to as a D-A junction. D-A junctions can be formed by a chromosomal tandem duplication (TD) of ABCD (FIG. 6A v) or when the ABCD allele is excised and circularized (FIG. 6A iv). FIG. 6B shows the periodic allele length distribution of apparent insertions, color-coded by whether or not they have a D-A junction. Across the 6 sperm samples, 96% of apparent insertions longer than 20 bp (307 out of 320) and 100% of apparent insertions longer than 125 bp (n=307) contain D-A junctions (FIG. 6B) and no D-A junctions were detected in matched blood samples (n=7 insertions >20 bp). The length distribution of D-A junction-containing apparent insertions is strikingly similar to that reported for smaller extrachromosomal circular DNAs (eccDNA), also called microDNAs, in various cell types including sperm.

To determine whether the D-A junctions detected by DS arose from microDNAs, the relationship between the lengths of DNA fragments containing D-A junctions and the length of ABCD alleles was explored. Genomic DNA was enzymatically fragmented during DS library preparation, and the size distribution of DNA fragments in final DS libraries was approximated by the insert size distribution of the sequenced libraries (insert size was calculated as the distance between the reference genome alignment of the 5′ ends of read 1 and read 2 in a read pair). If D-A junctions arise from chromosomal TDs, the size distribution of the fragments containing the junction should be independent of the size of the duplicated ABCD allele and similar to that of the whole library (FIG. 7A-7C). Additionally, any fragments larger than the ABCD allele would contain two copies of at least part of the ABCD sequence (e.g., CDABCD) and could also contain flanking non-ABCD sequence (FIG. 7A-7C). Conversely, circular DNA molecules must be cleaved one or more times to generate linear DNA fragments and the resulting DNA fragments in the final library will be shorter than or equal to the length of the DNA circle, which was defined as the length of the ABCD allele (FIG. 7A-7C).

To ensure a reasonable probability of detecting duplicated sequence if it was present, all D-A containing apparent insertions with an ABCD allele length of less than or equal to the median insert size of the 6 sperm libraries (233 bp) were selected (n=63) and the aligned duplex consensus reads supporting each call were manually reviewed. If most or all D-A containing fragments arose from chromosomal TDs, about half of fragments would be larger than 233 bp and half would be smaller (FIG. 6C, entire teal distribution, FIG. 7A-7C). Also, at least half of the events would have a fragment size larger than the ABCD allele length, and thus would include duplicated ABCD sequence and possibly non-ABCD flanking sequence (FIG. 7A-7C). Conversely, all microDNA-derived fragments would be smaller than or equal to 233 bp, smaller than or equal to the ABCD allele length, and would not contain any duplicated or flanking sequence (FIG. 6C, portion of distribution with diagonal hashes; FIG. 7A-7C). All 63 apparent insertions reviewed had fragment lengths less than 233 bp, which was highly inconsistent with D-A junctions arising primarily from chromosomal TDs (binomial p-value=1.08×10⁻¹⁹), and showed that most D-A junctions arose from microDNAs. Furthermore, 62 out of the 63 apparent insertions had fragment sizes shorter than their respective ABCD allele length, consistent with a microDNA origin. One chromosomal TD was identified conclusively: a 75 bp allele on a 180 bp fragment, containing duplicated and flanking sequence. Notably, this was the smallest D-A junction-containing variant call in the dataset and was much smaller than all other D-A junction-containing alleles (FIG. 6B).

All D-A junction-containing events with allele length greater than 125 bp were considered to be candidate, or putative, eccDNA. Thus, 307 candidate, or putative, eccDNA were detected across the 6 sperm samples (average 51 per sample, SD 18), yielding an average frequency of 41 (SD 14) unique DNA circles per 1 billion duplex bases in sperm, which is higher than all other large indel (>20 bp) and SV types combined. No candidate, or putative, eccDNA were identified in the 6 matching blood samples.

### Example 4. eccDNA Discovery Using Exonuclease Enrichment

DNA samples (HeLa (BioChain): DNA from HeLa cells, purchased from BioChain; Blood (TS1): DNA from human whole blood, extracted using Agilent DNA extraction kit; Sperm (TS2): DNA from human sperm, extracted from cells or tissue which were put in Qiagen RLT lysis buffer with 10% TCEP and homogenized by bead-beating, followed by DNA extraction performed using a modified Qiagen DNeasy mini protocol; and Sperm (TS3): DNA from human sperm, extracted from cells or tissue which were put in Qiagen RLT with 10% TCEP, followed by addition of Proteinase K to a final concentration of 200 μg/ml and incubation of samples for 2 hr at 56 C, followed by DNA extraction performed using a modified Qiagen DNeasy Mini protocol) were subjected to exonuclease treatment to deplete linear DNA, and therefore enrich any circular DNA species present in the sample. If the D-A junction-containing fragments detected by DS arise primarily or exclusively from DNA circles, exonuclease treatment would increase the frequency of events detected (frequency=(#circles detected)/(#duplex bases sequenced)). If D-A junction-containing fragments arose from chromosomal tandem duplications instead, exonuclease treatment would not increase their frequency.

1 ug aliquots of DNA were treated with E. coli Exonuclease V (RecBCD, NEB M0345L) according to the manufacturer-recommended protocol, but with varying incubation times. Three parallel reactions were pooled for each DNA sample and incubation time to ensure enough yield. No-enzyme control reactions were performed for the 30-minute timepoint (“control”). Following exonuclease treatment, DNA was purified with SPRI beads (1.2× ratio). Exonuclease treatment decreased total DNA mass between 85% and >99% for all sample types, relative to the no-enzyme controls. Enrichment of circular DNA was further confirmed by qPCR quantitation of the ratio of mitochondrial to nuclear DNA for HeLa and blood samples.

DS libraries were prepared with 300 ng DNA input for controls and between 2 and 150 ng input for exo-treated samples. The human mutagenesis panel (20×2.4 kb targets across the genome) was used for hybrid capture. Candidate circles were defined as “indel” variant calls (from standard DS variant calling) with the following additional characteristics:
the alt allele is longer than the reference allele and >125 bp; and
the variant call is consistent with the occurrence of a D-A junction in the consensus read pair.

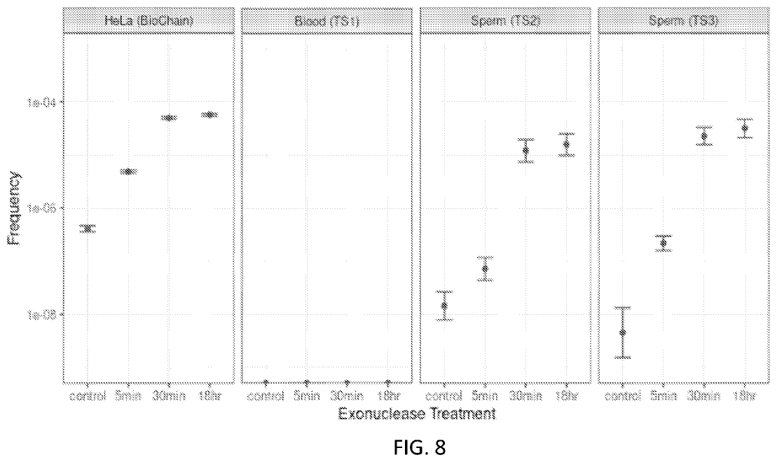

FIG. 8 shows the frequency of candidate circles, calculated as frequency=(#circles detected)/(#duplex bases sequenced). Exonuclease treatment increased the frequency of candidate circles in 3 of the 4 sample types tested, in a time-dependent manner. In the fourth sample type, blood from a healthy young donor, no candidate circles were detected in control or exonuclease treated samples. This finding shows that D-A junction-containing fragments detected by DS arise primarily or exclusively from DNA circles.

### Example 5. Putative eccDNA in Tumor and Normal Samples

Without wishing to be bound by a particular theory, the circular DNA profile (amount and characteristics of eccDNA and microDNA) can differ between normal and cancerous tissues, with the possible utility of circular DNA profiles as biomarkers to monitor cancer progression and post-treatment outcomes. If DS is detecting true biological differences in the frequency of circular DNA, differences are observable between paired tumor-normal samples.

Three pairs of matched tumor and normal DNA were purchased from BioChain, representing 3 common cancer types (breast, colon, lung). DS libraries were prepared with 750 ng of DNA input. The DuplexSeq Human Mutagenesis Assay panel (20×2.4 kb targets across the genome) was used for hybrid capture. Putative DNA circles are defined as “indel” variant calls (from standard DS variant calling) with the following additional characteristics:
the alt allele is longer than the reference allele and >125 bp; and
the variant call is consistent with the occurrence of a D-A junction in the consensus read pair.

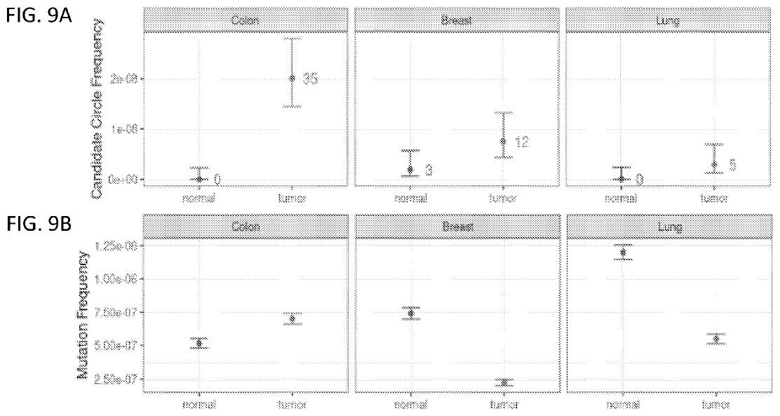

FIG. 9A-9B show the frequency of putative DNA circles, calculated as frequency=(#circles detected)/(#duplex bases sequenced). DS showed a moderate increase in putative DNA circles in all three tumors, compared to matched normal samples (FIG. 9A). The increase in putative DNA circles did not correspond to an increase in mutation frequency in two of the three tumor types (FIG. 9B).

### Example 6. eccDNA as a Measure of Clastogenicity

To assess whether DNA circle frequency is a proxy for clastogenicity, DS data for various genotoxic agents were re-analyzed. ENU is a well-known potent mutagen and clastogen. DS data show that compound #1 is non-mutagenic (at the tested doses) and compound #2 increases mutation frequency in a dose-dependent manner.

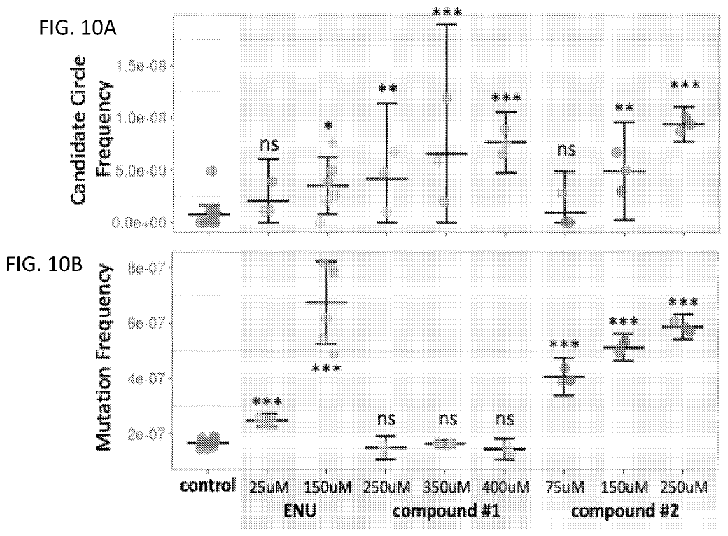

Human TK6 cells were treated with different potentially genotoxic compounds. DS libraries were prepared with 500 ng of DNA input. The human mutagenesis panel (20×2.4 kb targets across the genome) was used for hybrid capture. Putative circles were defined as “indel” variant calls (from standard DS variant calling) with an alt allele that is longer than the reference allele and >125 bp in length. In other data sets, all variants that satisfy this criterion were also shown to be associated with D-A junctions. FIG. 10A-10B show the frequency of putative circles, calculated as frequency=(#circles detected)/(#duplex bases sequenced).

Treatment with ENU, a known clastogen, increased candidate DNA circle frequency in a dose-dependent manner, reaching statistical significance in the highest dose group. Treatment with compounds #1 and #2 also caused a dose-dependent increase in the frequency of putative DNA circles, despite compound #1 being non-mutagenic at the doses tested.

In a separate experiment, TK6 cells were co-cultured with HepaRG cells in a system where cells are physically separated but culture media is shared. Co-cultures were treated with ultra-pure water (control) or cyclophosphamide, three replicates per group. DS libraries were prepared with variable input mass. The DuplexSeq Human Mutagenesis Assay panel (20×2.4 kb targets across the genome) was used for hybrid capture.

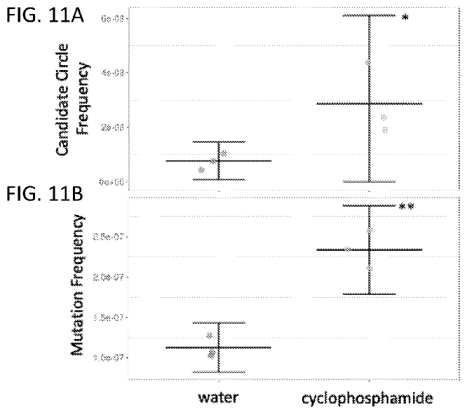

DNA circles were defined as “indel” variant calls (from standard DS variant calling) with an alt allele that is longer than the reference allele and >125 bp in length. In other data sets, all variants that satisfy this criterion were also shown to be associated with D-A junctions. FIG. 11A-11B show the frequency of DNA circles, calculated as frequency=(#circles detected)/(#duplex bases sequenced). The known clastogen, cyclophosphamide, significantly increased both DNA circle frequency and mutation frequency in a TK6-HepaRG co-culture system.

### X. CONCLUSION

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments. All references cited herein are incorporated by reference as if fully set forth herein.

1. A method of identifying at least one extrachromosomal circular DNA (eccDNA) in a sample comprising double-stranded DNA, the method comprising:
performing or having performed duplex sequencing on the sample, wherein the duplex sequencing comprises the steps of:
ligating adaptors to the ends of the double-stranded DNA, wherein at least one adaptor comprises a nucleotide sequence that tags a strand of the double-stranded DNA such that the strand of the double-stranded DNA has a distinctly identifiable nucleotide sequence relative to its complementary strand;
amplifying strands of the double-stranded DNA using the ligated adaptors to generate at least first strand amplicons and second strand amplicons;
sequencing at least the first strand amplicons and the second strand amplicons to produce a plurality of sequence reads comprising first strand sequence reads and second strand sequence reads; and
identifying or having identified the eccDNA using the plurality of sequence reads of the duplex sequencing.

2. The method of claim 1, wherein the performing or having performed duplex sequencing comprises:
generating an error-corrected sequence read by comparing the first strand sequence reads and second strand sequence reads by discounting nucleotide positions that do not agree.

3. The method of claim 1, wherein the identifying or having identified the eccDNA comprises:
identifying a subset of sequence reads from the plurality of sequence reads, wherein sequence reads of the subset of sequence reads each independently comprise a reference allele junction;
from amongst the subset of sequence reads, distinguishing putative eccDNA sequence reads from chromosomal tandem duplication sequence reads; and
determining a quantity of eccDNA according to the distinguished putative eccDNA sequence reads.

4. The method of claim 3, wherein the reference allele junction comprises a nucleic acid sequence D-A.

5. The method of claim 4, wherein the nucleic acid sequence D-A comprises in 5′ to 3′ direction a nucleic acid sequence D operatively conjugated to a nucleic acid sequence A.

6. The method of claim 4, wherein the nucleic acid sequence D is located downstream of the nucleic acid sequence A in a reference genomic locus of the reference allele.

7. The method of any one of claims 4-7, wherein the nucleic acid sequence D-A is at least 1 base pair (bp) in length.

8. The method of claim 3, wherein the reference allele junction is due to an apparent indel, or an apparent structural variant.

9. The method of claim 8, wherein the apparent indel is at least 1 bp in length.

10. The method of claim 8, wherein the apparent structural variant is due to an apparent insertion, or apparent duplication.

11. The method of claim 10, wherein the apparent structural variant due to the apparent insertion, or the apparent duplication, is at least 20 bp in length.

12. The method of any one of claims 1-11, wherein distinguishing putative eccDNA sequence reads from chromosomal tandem duplication sequence reads comprises the step of selecting a subset of D-A junction-containing consensus sequencing reads with allele length less than a threshold apparent insert size.

13. The method of any one of claims 1-12, wherein distinguishing putative eccDNA sequence reads from chromosomal tandem duplication sequence reads comprises the step of inferring the fragment size of each read pair in the subset and comparing it to the threshold apparent insert size, one by one, or comparing the distribution of inferred fragment sizes to the distribution of insert sizes in the entire library.

14. The method of any one of claims 1-13, wherein distinguishing putative eccDNA sequence reads from chromosomal tandem duplication sequence reads comprises the step of comparing the inferred fragment size of any one consensus read pair to the allele size of that read pair.

15. The method of any one of claims 1-14, wherein distinguishing putative eccDNA sequence reads from chromosomal tandem duplication sequence reads comprises the steps of
selecting a subset of D-A junction-containing consensus sequencing reads with allele length less than a threshold apparent insert size;
inferring the fragment size of each read pair in the subset and comparing it to the threshold apparent insert size, one by one, or comparing the distribution of inferred fragment sizes to the distribution of insert sizes in the entire library; and
comparing the inferred fragment size of any one consensus read pair to the allele.

16. The method of any one of claims 12-15, wherein the threshold apparent insert size is at least 20 bp in length.

17. The method of any one of claims 12-15, wherein the inferred fragment size is at least 20 bp in length.

18. The method of any one of claims 12-15, wherein the putative eccDNA is identified as having the inferred fragment size less than or equal to the allele length.

19. The method of claim 18, wherein the allele length is at least 20 bp.

20. The method of any one of claims 1-19, wherein the method comprises the steps of:
performing or having performed duplex sequencing on the sample, wherein the duplex sequencing comprises the steps of:
ligating adaptors to the ends of the double-stranded DNA, wherein at least one adaptor comprises a nucleotide sequence that tags a strand of the double-stranded DNA such that the strand of the double-stranded DNA has a distinctly identifiable nucleotide sequence relative to its complementary strand;
amplifying strands of the double-stranded DNA using the ligated adaptors to generate at least first strand amplicons and second strand amplicons;
sequencing at least the first strand amplicons and the second strand amplicons to produce a plurality of sequence reads comprising first strand sequence reads and second strand sequence reads;
generating an error-corrected sequence read by comparing the first strand sequence reads and second strand sequence reads by discounting nucleotide positions that do not agree; and
identifying or having identified the eccDNA using the plurality of sequence reads of the duplex sequencing, wherein the identifying or having identified the eccDNA comprises the steps of:
identifying a subset of sequence reads from the plurality of sequence reads, wherein sequence reads of the subset of sequence reads each independently comprise a reference allele junction;
from amongst the subset of sequence reads, distinguishing putative eccDNA sequence reads from chromosomal tandem duplication sequence reads, wherein distinguishing putative eccDNA sequence reads comprises the steps of:
selecting a subset of D-A junction-containing consensus sequencing reads with allele length less than a threshold apparent insert size;
inferring the fragment size of each read pair in the subset and comparing it to the threshold apparent insert size, one by one, or comparing the distribution of inferred fragment sizes to the distribution of insert sizes in the entire library; and
comparing the inferred fragment size of any one consensus read pair to the allele; and
determining a quantity of eccDNA according to the distinguished putative eccDNA sequence reads.

21. The method of claim 1 or 20, further comprising:
prior to performing or having performed duplex sequencing:
exposing one or more cells to a potential clastogen; and
obtaining the eccDNA from the one or more cells.

22. The method of claim 21, further comprising:
evaluating clastogenicity of the potential clastogen based on the determined profile of the eccDNA.

23. The method of claim 22, wherein the profile comprises any one, or any combination, of quantity, frequency, quality, size, genomic location, or any other characteristic of the eccDNA.

24. The method of claim 23, wherein the profile comprises frequency of the eccDNA.

25. The method of any one of claims 21-24, wherein the potential clastogen is a compound, a physical exposure, a biological agent, or a complex mixture and/or an environmental exposure.

26. The method of any one of claims 1-25, wherein the sample is selected from the group consisting of a sperm sample, semen sample, prostatic fluid sample, testicular biopsy sample, spermatogonia sample, germ cell sample, gamete sample, swab, lavage, aspirate, biopsy, tissue sample, tumor sample, preneoplastic sample, liquid biopsy, hyperplasia sample, hypertrophy sample, dysplastic sample, urine sample, CSF sample, any other body fluid sample, autopsy sample, necropsy sample, surgical sample, model organism sample, plasma sample, serum sample, gastric sample, bone marrow sample, stool sample, brushing sample, bile sample, pancreatic fluid sample, synovial fluid sample, sputum sample, mucus sample, vitreous sample, forensic sample, environmental sample, bacterial sample, fungal sample, mammalian sample, human sample, and diagnostic sample.

27. The method of any one of claims 1-26, wherein the sample comprises cancer cells or cancer-derived nucleic acids.

28. The method of any one of claims 1-27, wherein the biological sample comprises cell-free DNA.

29. The method of any one of claims 1-28, wherein the at least one adaptor sequence is or comprises at least one non-standard nucleotide.

30. The method of claim 29, wherein the non-standard nucleotide is selected from a uracil, a methylated nucleotide, an RNA nucleotide, a ribose nucleotide, an 8-oxo-guanine, a biotinylated nucleotide, a desthiobiotin nucleotide, a thiol modified nucleotide, an acrydite modified nucleotide an iso-dC. an iso dG, a 2′-0-methyl nucleotide, an inosine nucleotide Locked Nucleic Acid, a peptide nucleic acid, a 5 methyl dC, a 5-bromo deoxyuridine, a 2,6-Diaminopurine, 2-Aminopurine nucleotide, an abasic nucleotide, a 5-Nitroindole nucleotide, an adenylated nucleotide, an azide nucleotide, a digoxigenin nucleotide, an I-linker, a 5′ Hexynyl modified nucleotide, an 5-Octadiynyl dU, photocleavable spacer, a non-photocleavable spacer, a click chemistry compatible modified nucleotide, a fluorescent dye, biotin, furan, BrdU, Fluoro-dU, loto-dU, and any combination thereof.

31. The method of claim 1, wherein the method further comprises performing eccDNA enrichment.

32. The method of claim 31, wherein performing the eccDNA enrichment comprises:
performing a size selection;
performing an exonuclease treatment; and/or
using a DNA binding protein that will differentially bind to or remain bound to double-stranded circular DNA molecules as compared to double-stranded linear DNA molecules.

33. The method of claim 32, wherein the size selection comprises a use of paramagnetic beads, electrophoresis, column filtrations, density gradient centrifugation, or selective extraction, at a size threshold of about 10,000 bp.

34. The method of claim 32, wherein the exonuclease is selected from any one of exonuclease I, exonuclease T, exonuclease VII, exonuclease III, T7 exonuclease, exonuclease V (RecBCD), exonuclease VIII, lambda exonuclease, T5 exonuclease, and any combination thereof.

35. A method for identifying extrachromosomal circular DNA (eccDNA), the method comprising:
obtaining a plurality of sequence reads of double-stranded DNA sequenced using duplex sequencing;
identifying a subset of sequence reads from the plurality of sequence reads, wherein sequence reads of the subset of sequence reads each independently comprise a reference allele junction;
from amongst the subset of sequence reads, distinguishing putative eccDNA sequence reads from chromosomal tandem duplication sequence reads; and
identifying eccDNA according to the distinguished putative eccDNA sequence reads.

36. The method of claim 35, wherein the reference allele junction comprises a nucleic acid sequence D-A.

37. The method of claim 36, wherein the nucleic acid sequence D-A comprises in 5′ to 3′ direction a nucleic acid sequence D operatively conjugated to a nucleic acid sequence A.

38. The method of claim 37, wherein the nucleic acid sequence D is located downstream of the nucleic acid sequence A in a reference genomic locus of the reference allele.

39. The method of any one of claims 36-38, wherein the nucleic acid sequence D-A is at least 1 base pairs (bp) in length.

40. The method of claim 35, wherein the reference allele junction is due to an apparent indel, or an apparent structural variant.

41. The method of claim 40, wherein the apparent indel is at least 1 bp in length.

42. The method of claim 40, wherein the apparent structural variant is due to an apparent insertion, or an apparent duplication.

43. The method of claim 42, wherein the apparent structural variant due to the apparent insertion, or the apparent duplication, is at least 20 bp in length.

44. The method of any one of claims 35-43, wherein distinguishing putative eccDNA sequence reads from chromosomal tandem duplication sequence reads comprises the step of selecting a subset of D-A junction-containing consensus sequencing reads with allele length less than a threshold apparent insert size.

45. The method of any one of claims 35-44, wherein distinguishing putative eccDNA sequence reads from chromosomal tandem duplication sequence reads comprises the step of inferring the fragment size of each read pair in the subset and comparing it to the threshold apparent insert size, one by one, or comparing the distribution of inferred fragment sizes to the distribution of insert sizes in the entire library.

46. The method of any one of claims 35-45, wherein distinguishing putative eccDNA sequence reads from chromosomal tandem duplication sequence reads comprises the step of comparing the inferred fragment size of any one consensus read pair to the allele size of that read pair.

47. The method of any one of claims 35-46, wherein distinguishing putative eccDNA sequence reads from chromosomal tandem duplication sequence reads comprises the steps of:
selecting a subset of D-A junction-containing consensus sequencing reads with allele length less than a threshold apparent insert size;
inferring the fragment size of each read pair in the subset and comparing it to the threshold apparent insert size, one by one, or comparing the distribution of inferred fragment sizes to the distribution of insert sizes in the entire library; and
comparing the inferred fragment size of any one consensus read pair to the allele.

48. The method of any one of claims 44-47, wherein the threshold apparent insert size is at least 20 bp in length.

49. The method of any one of claims 44-47, wherein the inferred fragment size is at least 20 bp in length.

50. The method of any one of claims 44-47, wherein the putative eccDNA is identified as having the inferred fragment size less than or equal to the allele length.

51. The method of claim 50, wherein the allele length is at least 20 bp.

52. The method of claim 35, wherein the method comprises the steps of:
obtaining a plurality of sequence reads of double-stranded DNA sequenced using duplex sequencing;
identifying a subset of sequence reads from the plurality of sequence reads, wherein sequence reads of the subset of sequence reads each independently comprise a reference allele junction;
from amongst the subset of sequence reads, distinguishing putative eccDNA sequence reads from chromosomal tandem duplication sequence reads, wherein distinguishing putative eccDNA sequence reads comprises the steps of:
selecting a subset of D-A junction-containing consensus sequencing reads with allele length less than a threshold apparent insert size;
inferring the fragment size of each read pair in the subset and comparing it to the threshold apparent insert size, one by one, or comparing the distribution of inferred fragment sizes to the distribution of insert sizes in the entire library; and
comparing the inferred fragment size of any one consensus read pair to the allele; and
identifying eccDNA according to the distinguished putative eccDNA sequence reads.

53. A method of evaluating clastogenicity of a potential clastogen, the method comprising:
obtaining double-stranded DNA comprising putative extrachromosomal circular DNA (eccDNA) from one or more cells;
performing duplex sequencing of the double-stranded DNA to obtain a plurality of sequence reads of the double-stranded DNA;
identifying a subset of sequence reads from the plurality of sequence reads, wherein sequence reads of the subset of sequence reads each independently comprise a reference allele junction;
from amongst the subset of sequence reads, distinguishing putative eccDNA sequence reads from chromosomal tandem duplication sequence reads;
determining a profile of eccDNA according to the distinguished putative eccDNA sequence reads; and
evaluating clastogenicity of the potential clastogen according to the determined profile of the eccDNA.

54. The method of claim 53, wherein the one or more cells are one of more cells exposed to the potential clastogen, control, or untreated cells.

55. The method of claim 53, wherein the reference allele junction comprises a nucleic acid sequence D-A.

56. The method of claim 55, wherein the nucleic acid sequence D-A comprises in 5′ to 3′ direction a nucleic acid sequence D operatively conjugated to a nucleic acid sequence A.

57. The method of claim 56, wherein the nucleic acid sequence D is located downstream of the nucleic acid sequence A in a reference genomic locus of the reference allele.

58. The method of any one of claims 53-57, wherein the nucleic acid sequence D-A is at least 1 bp in length.

59. The method of claim 53, wherein the reference allele junction is due to an apparent indel, or an apparent structural variant.

60. The method of claim 59, wherein the apparent indel is at least 1 bp in length.

61. The method of claim 59, wherein the apparent structural variant is due to an apparent insertion, or an apparent duplication.

62. The method of claim 61, wherein the apparent structural variant due to the apparent insertion, or the apparent duplication, is at least 20 bp in length.

63. The method of any one of claims 53-62, wherein distinguishing putative eccDNA sequence reads from chromosomal tandem duplication sequence reads comprises the step of selecting a subset of D-A junction-containing consensus sequencing reads with allele length less than a threshold apparent insert size.

64. The method of any one of claims 53-63, wherein distinguishing putative eccDNA sequence reads from chromosomal tandem duplication sequence reads comprises the step of inferring the fragment size of each read pair in the subset and comparing it to the threshold apparent insert size, one by one, or comparing the distribution of inferred fragment sizes to the distribution of insert sizes in the entire library.

65. The method of any one of claims 53-64, wherein distinguishing putative eccDNA sequence reads from chromosomal tandem duplication sequence reads comprises the step of comparing the inferred fragment size of any one consensus read pair to the allele size of that read pair.

66. The method of any one of claims 53-65, wherein distinguishing putative eccDNA sequence reads from chromosomal tandem duplication sequence reads comprises the steps of:
selecting a subset of D-A junction-containing consensus sequencing reads with allele length less than a threshold apparent insert size;
inferring the fragment size of each read pair in the subset and comparing it to the threshold apparent insert size, one by one, or comparing the distribution of inferred fragment sizes to the distribution of insert sizes in the entire library; and
comparing the inferred fragment size of any one consensus read pair to the allele.

67. The method of any one of claims 62-66, wherein the threshold apparent insert size is at least 20 bp in length.

68. The method of any one of claims 62-66, wherein the inferred fragment size is at least 20 bp in length.

69. The method of any one of claims 62-66, wherein the putative eccDNA is identified as having the inferred fragment size less than or equal to the allele length.

70. The method of claim 69, wherein the allele length is at least 20 bp.

71. The method of claim 53, wherein the evaluating clastogenicity of the potential clastogen further comprises the step of comparing the eccDNA profiles from one or more cells exposed to the potential clastogen to control or untreated samples from the same cohort.

72. The method of claim 53, wherein the method comprises the steps of:
obtaining double-stranded DNA comprising putative extrachromosomal circular DNA (eccDNA) from one or more cells exposed to the potential clastogen;
performing duplex sequencing of the double-stranded DNA to obtain a plurality of sequence reads of the double-stranded DNA;
identifying a subset of sequence reads from the plurality of sequence reads, wherein sequence reads of the subset of sequence reads each independently comprise a reference allele junction;
from amongst the subset of sequence reads, distinguishing putative eccDNA sequence reads from chromosomal tandem duplication sequence reads, wherein the distinguishing putative eccDNA sequence reads comprises the steps of:
selecting a subset of D-A junction-containing consensus sequencing reads with allele length less than a threshold apparent insert size;
inferring the fragment size of each read pair in the subset and comparing it to the threshold apparent insert size, one by one, or comparing the distribution of inferred fragment sizes to the distribution of insert sizes in the entire library; and
comparing the inferred fragment size of any one consensus read pair to the allele;
determining a profile of eccDNA according to the distinguished putative eccDNA sequence reads; and
evaluating clastogenicity of the potential clastogen according to the determined profile of the eccDNA, wherein the evaluating clastogenicity of the potential clastogen further comprises the step of comparing the eccDNA profiles from one or more cells exposed to the potential clastogen to control or untreated samples from the same cohort.

73. The method of any one of claim 53 or 72, wherein the profile comprises any one, or any combination, of quantity, frequency, quality, size, genomic location, or any other characteristic of the eccDNA.

74. The method of claim 73, wherein the profile comprises frequency of the eccDNA.

75. The method of any one of claim 53 or 72, wherein the method comprises the step of distinguishing a tumor DNA from a paired normal DNA based on frequency of eccDNA in the tumor.

76. The method of any one of claim 53 or 72, wherein the method further comprises performing eccDNA enrichment.

77. The method of claim 76, wherein performing the eccDNA enrichment comprises:
performing a size selection;
performing an exonuclease treatment; and/or
using a DNA binding protein that will differentially bind to or remain bound to double-stranded circular DNA molecules as compared to double-stranded linear DNA molecules.

78. The method of claim 77, wherein the size selection comprises a use of paramagnetic beads, electrophoresis, column filtrations, density gradient centrifugation, or selective extraction, at a size threshold of about 10,000 bp.

79. The method of claim 77, wherein the exonuclease is selected from any one of exonuclease I, exonuclease T, exonuclease VII, exonuclease III, T7 exonuclease, exonuclease V (RecBCD), exonuclease VIII, lambda exonuclease, T5 exonuclease, and any combination thereof.

80. The method of any one of claims 53-79, wherein the potential clastogen is a compound, a physical exposure, a biological agent, or a complex mixture and/or an environmental exposure.

81. A method of evaluating clastogenicity of a potential clastogen, the method comprising:
obtaining double-stranded DNA from one or more cells;
performing duplex sequencing of the double-stranded DNA to obtain a plurality of sequence reads of the double-stranded DNA;
identifying a subset of sequence reads from the plurality of sequence reads, wherein sequence reads of the subset of sequence reads each independently comprise a reference allele junction;
from amongst the subset of sequence reads, distinguishing putative eccDNA sequence reads from chromosomal tandem duplication sequence reads; and
evaluating clastogenicity of the potential clastogen according to the distinguished putative eccDNA.

82. The method of claim 81, wherein the one or more cells do not comprise eccDNA.

83. The method of claim 81, wherein the one or more cells are one of more cells exposed to the clastogen, control, or untreated cells.

84. The method of claim 81, wherein the reference allele junction comprises a nucleic acid sequence D-A.

85. The method of claim 84, wherein the nucleic acid sequence D-A comprises in 5′ to 3′ direction a nucleic acid sequence D operatively conjugated to a nucleic acid sequence A.

86. The method of claim 85, wherein the nucleic acid sequence D is located downstream of the nucleic acid sequence A in a reference genomic locus of the reference allele.

87. The method of any one of claims 84-86, wherein the nucleic acid sequence D-A is at least 1 bp in length.

88. The method of claim 87, wherein the reference allele junction is due to an apparent indel, or an apparent structural variant.

89. The method of claim 88, wherein the apparent indel is at least 1 bp in length.

90. The method of claim 87, wherein the apparent structural variant is due to an apparent insertion, or an apparent duplication.

91. The method of claim 88, wherein the apparent structural variant due to the apparent insertion, or the apparent duplication, is at least 20 bp in length.

92. The method of any one of claims 81-91, wherein distinguishing putative eccDNA sequence reads from chromosomal tandem duplication sequence reads comprises the steps of:
selecting a subset of D-A junction-containing consensus sequencing reads with allele length less than a threshold apparent insert size;
inferring the fragment size of each read pair in the subset and comparing it to the threshold apparent insert size, one by one, or comparing the distribution of inferred fragment sizes to the distribution of insert sizes in the entire library; and
comparing the inferred fragment size of any one consensus read pair to the allele.

93. The method of any one of claims 81-92, wherein the threshold apparent insert size is at least 20 bp in length.

94. The method of any one of claims 81-93, wherein the inferred fragment size is at least 20 bp in length.

95. The method of claim 81, wherein the evaluating clastogenicity of the potential clastogen further comprises the step of comparing the eccDNA profiles from one or more cells exposed to the potential clastogen to control or untreated samples from the same cohort.

96. The method of claim 81, wherein the method comprises the steps of:
obtaining double-stranded DNA from one or more cells;
performing duplex sequencing of the double-stranded DNA to obtain a plurality of sequence reads of the double-stranded DNA;
identifying a subset of sequence reads from the plurality of sequence reads, wherein sequence reads of the subset of sequence reads each independently comprise a reference allele junction;
from amongst the subset of sequence reads, distinguishing putative eccDNA sequence reads from chromosomal tandem duplication sequence reads, wherein the distinguishing putative eccDNA sequence reads comprises the steps of:
selecting a subset of D-A junction-containing consensus sequencing reads with allele length less than a threshold apparent insert size;
inferring the fragment size of each read pair in the subset and comparing it to the threshold apparent insert size, one by one, or comparing the distribution of inferred fragment sizes to the distribution of insert sizes in the entire library; and
comparing the inferred fragment size of any one consensus read pair to the allele;
evaluating clastogenicity of the potential clastogen according to the distinguished putative eccDNA.

97. The method of claim 81, wherein the method further comprises performing eccDNA enrichment.

98. The method of claim 97, wherein performing the eccDNA enrichment comprises:
performing a size selection;
performing an exonuclease treatment; and/or
using a DNA binding protein that will differentially bind to or remain bound to double-stranded circular DNA molecules as compared to double-stranded linear DNA molecules.

99. The method of claim 98, wherein the size selection comprises a use of paramagnetic beads, electrophoresis, column filtrations, density gradient centrifugation, or selective extraction at a size threshold of about 10,000 bp.

100. The method of claim 98, wherein the exonuclease is selected from any one of exonuclease I, exonuclease T, exonuclease VII, exonuclease III, T7 exonuclease, exonuclease V (RecBCD), exonuclease VIII, lambda exonuclease, T5 exonuclease, and any combination thereof.

101. The method of any one of claims 81-100, wherein the potential clastogen is a compound, a physical exposure, a biological agent, or a complex mixture and/or an environmental exposure.

102. A method of evaluating genotoxicity, the method comprising:
a) evaluating clastogenicity comprising the steps of:
obtaining double-stranded DNA from one or more cells exposed to a potential genotoxin;
performing duplex sequencing of the double-stranded DNA to obtain a plurality of sequence reads of the double-stranded DNA;
identifying a subset of sequence reads from the plurality of sequence reads, wherein sequence reads of the subset of sequence reads each independently comprise a reference allele junction;
from amongst the subset of sequence reads, distinguishing putative eccDNA sequence reads from chromosomal tandem duplication sequence reads;
determining a profile of eccDNA according to the distinguished putative eccDNA sequence reads; and
evaluating clastogenicity of the potential genotoxin according to the determined profile of the eccDNA; and
b) evaluating mutagenicity comprising the steps of:
obtaining double-stranded DNA from one or more cells exposed to a potential genotoxin;
performing duplex sequencing of the double-stranded DNA to obtain a plurality of sequence reads of the double-stranded DNA;
determining a mutation profile of the double-stranded DNA; and
evaluating mutagenicity of the potential genotoxin according to the determined mutation profile of the double-stranded DNA.

103. The method of claim 102, wherein the reference allele junction comprises a nucleic acid sequence D-A.

104. The method of claim 103, wherein the nucleic acid sequence D-A comprises in 5′ to 3′ direction a nucleic acid sequence D operatively conjugated to a nucleic acid sequence A.

105. The method of claim 104, wherein the nucleic acid sequence D is located downstream of the nucleic acid sequence A in a reference genomic locus of the reference allele.

106. The method of any one of claims 103-105, wherein the nucleic acid sequence D-A is at least 1 base pairs (bp) in length.

107. The method of claim 102, wherein the reference allele junction is due to an apparent indel, or an apparent structural variant.

108. The method of claim 107, wherein the apparent indel is at least 1 bp in length.

109. The method of claim 107, wherein the apparent structural variant is due to an apparent insertion, or an apparent duplication.

110. The method of claim 109, wherein the apparent structural variant due to the apparent insertion, or the apparent duplication, is at least 20 bp in length.

111. The method of any one of claims 102-110, wherein distinguishing putative eccDNA sequence reads from chromosomal tandem duplication sequence reads comprises the step of selecting a subset of D-A junction-containing consensus sequencing reads with allele length less than a threshold insert size.

112. The method of any one of claims 102-111, wherein distinguishing putative eccDNA sequence reads from chromosomal tandem duplication sequence reads comprises the step of inferring the fragment size of each read pair in the subset and comparing it to the threshold insert size, one by one, or comparing the distribution of inferred fragment sizes to the distribution of insert sizes in the entire library.

113. The method of any one of claims 102-112, wherein distinguishing putative eccDNA sequence reads from chromosomal tandem duplication sequence reads comprises the step of comparing the inferred fragment size of any one consensus read pair to the allele size of that read pair.

114. The method of any one of claims 102-113, wherein distinguishing putative eccDNA sequence reads from chromosomal tandem duplication sequence reads comprises the steps of:
selecting a subset of D-A junction-containing consensus sequencing reads with allele length less than a threshold insert size;
inferring the fragment size of each read pair in the subset and comparing it to the threshold insert size, one by one, or comparing the distribution of inferred fragment sizes to the distribution of insert sizes in the entire library; and
comparing the inferred fragment size of any one consensus read pair to the allele.

115. The method of any one of claims 111-114, wherein the threshold apparent insert size is at least 20 bp in length.

116. The method of any one of claims 111-115, wherein the inferred fragment size is at least 20 bp in length.

117. The method of any one of claims 111-116, wherein the putative eccDNA is identified as having the inferred fragment size less than or equal to the allele length.

118. The method of claim 117, wherein the allele length is at least 20 bp.

119. The method of claim 102, wherein the profile comprises any one, or any combination, of quantity, frequency, quality, size, genomic location, or any other characteristic of the eccDNA.

120. The method of claim 119, wherein the profile comprises frequency of the eccDNA.

121. The method of claim 120, wherein the method further comprises performing eccDNA enrichment.

122. The method of claim 102, the method comprising the steps of:
a) evaluating clastogenicity comprising the steps of:
obtaining double-stranded DNA from one or more cells exposed to a potential genotoxin;
performing duplex sequencing of the double-stranded DNA to obtain a plurality of sequence reads of the double-stranded DNA;
identifying a subset of sequence reads from the plurality of sequence reads, wherein sequence reads of the subset of sequence reads each independently comprise a reference allele junction;
from amongst the subset of sequence reads, distinguishing putative eccDNA sequence reads from chromosomal tandem duplication sequence reads;
determining a profile of eccDNA according to the distinguished putative eccDNA sequence reads, wherein distinguishing putative eccDNA sequence reads comprises the steps of:
selecting a subset of D-A junction-containing consensus sequencing reads with allele length less than a threshold insert size;
inferring the fragment size of each read pair in the subset and comparing it to the threshold insert size, one by one, or comparing the distribution of inferred fragment sizes to the distribution of insert sizes in the entire library; and
comparing the inferred fragment size of any one consensus read pair to the allele; and
evaluating clastogenicity of the potential genotoxin according to the determined profile of the eccDNA; and
b) evaluating mutagenicity comprising the steps of:
obtaining double-stranded DNA from one or more cells exposed to a potential genotoxin;
performing duplex sequencing of the double-stranded DNA to obtain a plurality of sequence reads of the double-stranded DNA;
determining a mutation profile of the double-stranded DNA; and
evaluating mutagenicity of the potential genotoxin according to the determined mutation profile of the double-stranded DNA.

123. The method of any one of claim 102 or 122, wherein the performing eccDNA enrichment comprises:
performing a size selection;
performing an exonuclease treatment; and/or
using a DNA binding protein that will differentially bind to or remain bound to double-stranded circular DNA molecules as compared to double-stranded linear DNA molecules.

124. The method of claim 123, wherein the size selection comprises a use of paramagnetic beads, electrophoresis, column filtrations, density gradient centrifugation, or selective extraction, at a size threshold of about 10,000 bp.

125. The method of claim 123, wherein the exonuclease is selected from any one of exonuclease I, exonuclease T, exonuclease VII, exonuclease III, T7 exonuclease, exonuclease V (RecBCD), exonuclease VIII, lambda exonuclease, T5 exonuclease, and any combination thereof.

126. The method of any one of claims 102-125, wherein the xenobiotic is selected from any one of environmental pollutants, hydrocarbons, food additives, oil mixtures, pesticides, otherxenobiotics, synthetic polymers, carcinogens, drugs, antioxidants, and any combination thereof.

127. A method of assessing cancer risk in a sample, the method comprising:
obtaining double-stranded DNA comprising putative extrachromosomal circular DNA (eccDNA) from the sample;
performing duplex sequencing of the double-stranded DNA to obtain a plurality of sequence reads of the double-stranded DNA;
identifying a subset of sequence reads from the plurality of sequence reads, wherein sequence reads of the subset of sequence reads each independently comprise a reference allele junction;
from amongst the subset of sequence reads, distinguishing putative eccDNA sequence reads from chromosomal tandem duplication sequence reads;
determining a profile of eccDNA according to the distinguished putative eccDNA sequence reads; and
evaluating cancer risk of the sample according to the determined profile of the eccDNA.

128. The method of claim 127, wherein the reference allele junction comprises a nucleic acid sequence D-A.

129. The method of claim 128, wherein the nucleic acid sequence D-A comprises in 5′ to 3′ direction a nucleic acid sequence D operatively conjugated to a nucleic acid sequence A.

130. The method of claim 129, wherein the nucleic acid sequence D is located downstream of the nucleic acid sequence A in a reference genomic locus of the reference allele.

131. The method of any one of claims 128-130, wherein the nucleic acid sequence D-A is at least 1 base pairs (bp) in length.

132. The method of claim 127, wherein the reference allele junction is due to an apparent indel, or an apparent structural variant.

133. The method of claim 132, wherein the apparent indel is at least 1 bp in length.

134. The method of claim 132, wherein the apparent structural variant is due to an apparent insertion, or an apparent duplication.

135. The method of claim 134, wherein the apparent structural variant due to the apparent insertion, or the apparent duplication, is at least 20 bp in length.

136. The method of any one of claims 127-135, wherein distinguishing putative eccDNA sequence reads from chromosomal tandem duplication sequence reads comprises the step of selecting a subset of D-A junction-containing consensus sequencing reads with allele length less than a threshold insert size.

137. The method of any one of claims 127-136, wherein distinguishing putative eccDNA sequence reads from chromosomal tandem duplication sequence reads comprises the step of inferring the fragment size of each read pair in the subset and comparing it to the threshold insert size, one by one, or comparing the distribution of inferred fragment sizes to the distribution of insert sizes in the entire library.

138. The method of any one of claims 127-137, wherein distinguishing putative eccDNA sequence reads from chromosomal tandem duplication sequence reads comprises the step of comparing the inferred fragment size of any one consensus read pair to the allele size of that read pair.

139. The method of any one of claims 127-138, wherein distinguishing putative eccDNA sequence reads from chromosomal tandem duplication sequence reads comprises the steps of:
selecting a subset of D-A junction-containing consensus sequencing reads with allele length less than a threshold insert size;
inferring the fragment size of each read pair in the subset and comparing it to the threshold insert size, one by one, or comparing the distribution of inferred fragment sizes to the distribution of insert sizes in the entire library; and
comparing the inferred fragment size of any one consensus read pair to the allele.

140. The method of any one of claims 139-139, wherein the threshold apparent insert size is at least 20 bp in length.

141. The method of any one of claims 136-139, wherein the inferred fragment size is at least 20 bp in length.

142. The method of any one of claims 127-141, wherein the putative eccDNA is identified as having the inferred fragment size less than or equal to the allele length.

143. The method of claim 142, wherein the allele length is at least 20 bp.

144. The method of claim 127, wherein the profile comprises any one, or any combination, of quantity, frequency, quality, size, genomic location, or any other characteristic of the eccDNA.

145. The method of claim 144, wherein the profile comprises frequency of the eccDNA.

146. The method of claim 127, wherein the method comprises the steps of:
obtaining double-stranded DNA comprising putative extrachromosomal circular DNA (eccDNA) from the sample;
performing duplex sequencing of the double-stranded DNA to obtain a plurality of sequence reads of the double-stranded DNA;
identifying a subset of sequence reads from the plurality of sequence reads, wherein sequence reads of the subset of sequence reads each independently comprise a reference allele junction;
from amongst the subset of sequence reads, distinguishing putative eccDNA sequence reads from chromosomal tandem duplication sequence reads, wherein distinguishing putative eccDNA sequence reads comprises the steps of:
selecting a subset of D-A junction-containing consensus sequencing reads with allele length less than a threshold insert size;
inferring the fragment size of each read pair in the subset and comparing it to the threshold insert size, one by one, or comparing the distribution of inferred fragment sizes to the distribution of insert sizes in the entire library; and
comparing the inferred fragment size of any one consensus read pair to the allele;
determining a profile of eccDNA according to the distinguished putative eccDNA sequence reads; and
evaluating cancer risk of the sample according to the determined profile of the eccDNA.

147. The method of any one of claim 127 or 146, wherein the method further comprises performing eccDNA enrichment.

148. The method of claim 147, wherein the performing eccDNA enrichment comprises:
performing a size selection;
performing an exonuclease treatment; and/or
using a DNA binding protein that will differentially bind to or remain bound to double-stranded circular DNA molecules as compared to double-stranded linear DNA molecules.

149. The method of claim 148, wherein the size selection comprises a use of paramagnetic beads, electrophoresis, column filtrations, density gradient centrifugation, or selective extraction, at a size threshold of about 10,000 bp.

150. The method of claim 148, wherein the exonuclease is selected from any one of exonuclease I, exonuclease T, exonuclease VII, exonuclease III, T7 exonuclease, exonuclease V (RecBCD), exonuclease VIII, lambda exonuclease, T5 exonuclease, and any combination thereof.

151. The method of claim 127, wherein the sample is selected from the group consisting of a sperm sample, semen sample, prostatic fluid sample, testicular biopsy sample, spermatogonia sample, germ cell sample, gamete sample, swab, lavage, aspirate, biopsy, tissue sample, tumor sample, preneoplastic sample, liquid biopsy, hyperplasia sample, hypertrophy sample, dysplastic sample, urine sample, CSF sample, any other body fluid sample, autopsy sample, necropsy sample, surgical sample, model organism sample, plasma sample, serum sample, gastric sample, bone marrow sample, stool sample, brushing sample, bile sample, pancreatic fluid sample, synovial fluid sample, sputum sample, mucus sample, vitreous sample, forensic sample, environmental sample, bacterial sample, fungal sample, mammalian sample, human sample, and diagnostic sample.

152. The method of claim 127, wherein the sample is a cancerous sample, or a healthy sample.

153. The method of 127, wherein the evaluating cancer risk of the sample according to the determined profile of the eccDNA further comprises the step of comparing the eccDNA profiles to known eccDNA profiles.
